(19) United States
(12) Patent Application Publication
(10) Pub. No.: US 2024/0319522 A1
(43) Pub. Date: Sep. 26, 2024

(54) OPTICAL ELEMENT AND GLASSES

(71) Applicant: ELCYO CO., LTD., Kyoto (JP)

(72) Inventors: Sunri LEE, Kyoto (JP); Giichi SHIBUYA, Kyoto (JP); Hirohiko INA, Hyogo (JP); Hiroto YOSHII, Tokyo (JP)

(21) Appl. No.: 18/734,945

(22) Filed: Jun. 5, 2024

Related U.S. Application Data
(63) Continuation of application No. PCT/JP2022/045414, filed on Dec. 9, 2022.

(30) Foreign Application Priority Data
Dec. 11, 2021 (JP) 2021-201296
Sep. 29, 2022 (JP) 2022-155626

(51) Int. Cl. G02C 7/08; G02F 1/29

(52) U.S. Cl. CPC G02C 7/083; G02F 1/294

(57) ABSTRACT

This optical element has a first liquid crystal element LF1 and a second liquid crystal element LF3 that are each able to generate a refractive index distribution of a Fresnel cylindrical lens. The first liquid crystal element LF1 generates a linear focus axis in a first direction, and the second liquid crystal element LF3 generates a linear focus axis in a second direction. The first liquid crystal element LF1 and the second liquid crystal element LF3 are disposed so as to overlap each other such that the first direction and the second direction form an angle so as not to form a substantially perpendicular angle therebetween.

### RELATED APPLICATIONS

This application claims priority under 35 U.S.C. § 120 to International Patent Application No. PCT/JP2022/045414, which was filed on Dec. 9, 2022, and which claims priority under 35 U.S.C. § 119 to Japan Patent Application No. 2021-201296, which was filed on Dec. 11, 2021, and Japan Patent Application No. 2022-155626, which was filed on Sep. 29, 2022. The complete disclosures of the above-identified patent applications are hereby incorporated by reference.

### TECHNICAL FIELD

The present invention relates to optical elements and eyeglasses.

### BACKGROUND ART

Research on applying variable focus lenses to eyeglasses has been progressing for some time.

Patent Document 1 (JP Kokai (Unexamined Patent Publication) No. 2019-002977) describes eyeglasses having an optical element that can adjust the focal length by controlling the refractive index of a liquid crystal layer.

Further, Patent Document 2 (JP Kokai (Unexamined Patent Publication) No. 2019-195591) describes that eyewear is provided with a biological information detection device including an electro-oculography sensor that detects electro-oculography, an acceleration sensor, and a gyro sensor.

Further, Patent Document 3 (JP Kokai (Unexamined Patent Publication) No. 2018-010603) describes that a lens sales system is provided that allows a user to obtain power information of a lens used for eye examination.

Further, Patent Document 4 (JP Kohyo (National Publication of Translation version) No. 2019-530044) discloses the use of a smart device application to prevent deterioration of overall health, with emphasis on eye health (myopia).

Further, Patent Document 5 (JP Kokai (Unexamined Patent Publication) No. 2021-056985) describes a learning model generation method that can be expected to support the selection of eyeglass lenses by estimating the specifications of eyeglass lenses suitable for the user.

### SUMMARY OF THE INVENTION

### Technical Problem

As conventional eyeglasses with fixed power lenses, there are cases in which a single pair of glasses is worn and removed in daily life, and a plurality of pairs of glasses are used depending on the situation.

In addition, changes in visual acuity over time often occur due to factors such as lifestyle habits, growth, and aging. It will happen. If you neglect to buy new lenses and ignore changes in your vision, your quality of life will deteriorate, but if your vision changes rapidly, you will need to purchase lenses with a new prescription again in a short period of time.

In view of the problems with conventional eyeglasses as described above, an object of the present invention is to provide an optical element and eyeglasses that can improve user convenience and quality of life.

### Solution to Problem

The present invention can be understood from various aspects, and examples of these aspects are as follows.

(1) In view of the above problems, the glasses according to the present invention include a variable focus lens and a storage unit that stores power model information for changing the refractive index distribution of the variable focus lens according to the user's situation and behavior, and a control unit that controls the refractive index distribution of the variable focus lens based on the power model information.

(2) An eyeglass system including the eyeglasses of (1) and a server device, wherein the eyeglasses include a transmitter that transmits the information acquired by the eyeglasses, and the server device includes a recording unit that records acquired information, a diagnostic information recording unit that records diagnostic information regarding a user's predetermined disease, and a learning data construction unit that creates a learning data-set that includes the acquired information and diagnostic information. The server device further includes a model generation unit that uses machine learning to generate a learning model that outputs information related to prediction of diagnosis details for a predetermined disease. Machine learning is performed using acquired information included in a learning data-set as input data and diagnostic information included in the learning data-set as training data.

(3) The eyeglass system according to (2) may be characterized in that the acquired information includes information indicating control details of the variable focus lens.

(4) In the eyeglass system of (2), the server device may further include an optometry information storage unit that stores optometry information, and the learning data-set and the input data may include the optometry information.

(5) The program according to the present invention may be characterized by causing the computer to record the information acquired by the glasses in (1), and record the diagnostic information regarding the user's predetermined disease, and to construct a learning data-set including the acquired information and diagnostic information. Furthermore, the program causes a computer to execute a process of generating, by machine learning, a learning model that outputs information related to prediction of diagnosis contents for a predetermined disease. The machine learning is executed using acquired information included in a learning data-set as input data and diagnostic information included in the learning data-set as teacher data.

(6) An eyeglass system comprising the eyeglasses of (1) and a server device, wherein the eyeglasses further include an update control unit that updates power model information, and a transmitter that transmits the power model information updated by the update control unit. The server device includes a power model information storage unit that stores power model information associated with the user's identification information, and a diagnostic information storage unit that stores diagnostic information regarding a predetermined disease of the user. Furthermore, the server device has a learning data construction unit that constructs a learning data-set including power model information and diagnostic information, and a model generation unit that generates a learning model by machine learning using the power model information included in the learning data-set as input data and the diagnostic information included in the learning data-set as teacher data. Then, in the eyeglass system, it may also be characterized in that the power model information storage unit stores the updated power model information together with the power model information before the update, and the input data includes the current power model information and the previously stored some power model information.

(7) The program according to the present invention may be characterized by causing the computer to store the power model information that is associated with the user's identification information and is transmitted from the glasses in (1) in a power model information storage unit, and to store the diagnostic information regarding the user's predetermined disease, and to construct a learning data-set including the power model information and diagnostic information. Furthermore, the program causes a computer to execute a process of generating a learning model that outputs information related to prediction of diagnosis contents for a predetermined disease, by machine learning using the power model information included in the learning data-set as input data and the diagnostic information included in the learning data-set as teacher data Then, it may also be characterized in that the power model information storage unit stores the updated power model information in the eyeglass together with the power model information before the update, and the input data includes the current power model information and the previously stored some power model information.

(8) The learning model generation method according to the present invention includes the step of accumulating and storing the power model information transmitted from the glasses in (1) in association with the user's identification information in a power model information storage unit: a step of storing diagnostic information about the predetermined disease: a step of constructing a learning data-set including the power model information and the diagnostic information: a step of generating a learning model that outputs information related to predicting diagnosis details for the predetermined disease, by machine learning using the power model information included in the learning data-set as input data and the diagnostic information included in the learning data-set as teacher data, and it may also be characterized in that the power model information storage unit stores the updated power model information in the eyeglass together with the power model information before the update, and the input data includes the current power model information and the previously stored some power model information.

(9) An eyeglass system comprising the eyeglasses of (1) and a server device, wherein the eyeglasses include an update control unit that updates the power model information, and a transmitter that transmits the power model information updated by the update control unit. The server device includes a power model information storage unit that stores power model information associated with the user's identification information and a learning data construction unit that constructs a learning data-set including power model information, and a model generation unit that generates a learning model for outputting information regarding the power model information that can be adapted to the current user, by machine learning using the power model information included in the learning data-set as input data and teacher data. Then, in the eyeglass system, it may also be characterized in that the power model information storage unit stores the updated power model information together with the power model information before the update, and the input data includes a plurality of the power model information, and the teacher data includes the power model information accumulated after the plurality of power model information included in the input data.

(10) The program according to the present invention may be characterized by causing the computer to store the power model information that is associated with the user's identification information transmitted from the glasses in (1) in a power model information storage unit, and to construct a learning data-set including the power model information. Furthermore, the program causes a computer to execute a process of generating a learning model that outputs information regarding the power model information that can be adapted to the current user, by machine learning using the power model information included in the learning data-set as input data and teacher data.

Then, the program may also be characterized stores, in the power model information storage unit, power model information updated on the glasses together with power model information before update, and the input data includes a plurality of pieces of the power model information. Furthermore, the teacher data may include power model information accumulated after a plurality of pieces of power model information included in the input data.

(11) The learning model generation method according to the present invention includes the step of accumulating and storing the power model information transmitted from the glasses in (1) in association with the user's identification information in a power model information storage unit: a step of constructing a learning data-set including the power model information; and a step of generating a learning model for outputting information regarding the power model information that can be adapted to the current user, by machine learning using the power model information included in the learning data-set as input data and teacher data, and it may also be characterized in that the power model information storage unit stores the power model information updated in the eyeglasses together with the power model information before being updated in the eyeglasses and the input data includes a plurality of the power model information, and the teacher data includes the power model information accumulated after the plurality of power model information included in the input data.

(12) An eyeglass system comprising the eyeglasses of (1) and a server device, wherein the eyeglasses include an update control unit that updates the power model information and a transmitting unit configured to transmit at least part of the information, the at least part of the information includes power setting information that indicates a refractive index distribution that is set to the variable focus lens when the user is placed in a predetermined situation. The server device includes a power setting information storage unit that stores the power setting information transmitted from the transmitting unit in association with user identification information, and a diagnostic information storage unit storing diagnostic information regarding a user's predetermined disease, and a learning data construction unit that constructs a learning data-set including the power setting information and the diagnostic information and a model generation unit that generates a learning model that outputs information regarding prediction of diagnosis content for the predetermined disease, by machine learning using the power model information included in the learning data-set as input data and the diagnostic information included in the learning data as teacher data. Then, in the eyeglass system, it may also be characterized in that the power setting information storage unit accumulates and stores both the power setting information included in the updated power model information and the power setting information included in the power model information before update, and the input data includes the current power setting information and some previously accumulated power setting information.

(13) An eyeglass system comprising the eyeglasses of (1) and a server device, wherein the eyeglasses include an update control unit that updates the power model information and a transmitting unit configured to transmit at least part of the information, the at least part of the information includes power setting information that indicates a refractive index distribution that is set to the variable focus lens when the user is placed in a predetermined situation. The server device includes a power setting information storage unit that stores the power setting information transmitted from the transmitting unit in association with user identification information and a learning data construction unit that constructs a learning data-set including the power setting information storage unit, and a model generation unit that generates a learning model for outputting information regarding the power model information that is suitable for the current user by machine learning using the power setting information included in the learning data-set as input data and teacher data. Then, in the eyeglass system, it may also be characterized in that the power setting information storage unit accumulates and stores both the power setting information included in the updated power model information and the power setting information included in the power model information before update, and the input data includes a plurality of the power setting information, and the teacher data includes the power setting information accumulated after the plurality of power setting information included in the input data.

(14) An eyeglass system comprising the eyeglasses of (1) and a server device, wherein the eyeglasses include an update control unit that updates the power model information, and a transmission unit that transmits information acquired in the eyeglasses. The server device further includes a recording unit that records the acquired information, an optometry information recording unit that accumulates and records user's optometry information, and a learning data construction unit that constructs a learning data-set including the acquired information and the optometry information, and a model generation unit that generates a learning model for outputting information regarding the power model information that is suitable for the current user by machine learning based on the learning data-set. Then, in the eyeglass system, it may also be characterized in that the input data in machine learning based on the learning data-set includes a plurality of the optometry information and the acquired information, and the teacher data includes the optometric information accumulated after the plurality of optometric information included in the input data.

(15) The present invention is characterized by an eyeglass system, in which comprising eyeglasses having variable focus lenses and an operation device that accepts user input regarding the variable focus lenses, the operating device includes means for executing a mode that accepts input of settings regarding the astigmatic axis, and display control means for causing the display unit to display an image including a figure indicating the direction of the astigmatic axis in the mode.

(16) The present invention is characterized by eyeglasses, in which having variable focus lens configured to be able to control the direction of an astigmatic axis, and a display unit that superimposes and displays images or information on the real world, or a display unit that is arranged to cover the wearer's eyes, further, the eyeglasses are characterized by including means for executing a mode that accepts input from a user regarding the direction of the astigmatic axis of the variable focus lens, and a display control unit that causes the display unit to generate an image including a graphic indicating the direction of the astigmatic axis in the mode.

(17) The present invention provides a program for causing a computer to function as eyeglasses having variable focus lenses configured to be able to control the direction of an astigmatic axis, and a display unit that superimposes and displays images or information on the real world, or a display unit that is arranged to cover the wearer's eyes, further, the program are characterized by executing a mode that accepts input from a user regarding the direction of the astigmatic axis of the variable focus lens, a display control means for displaying an image including a figure indicating the direction of an astigmatic axis on the display unit in the mode.

(18) An eyeglass system including the glasses of (1) and an operating device that accepts user input for updating the power model information, and the eyeglass system, it may also be characterized in that the operating device includes means for executing an update support mode that supports updating of the power model information, wherein in the update support mode, a preselected parameter among the parameters in the power setting of the variable focus lens are controlled to change in stages according to a user's input.

(19) The present invention is characterized by eyeglasses, in which include two liquid crystal elements each having a plurality of unit-electrodes for generating a potential distribution in a liquid crystal layer, and a lens control device that outputs a control signal to each of the plurality of unit-electrodes of the two liquid crystal elements, and in the two liquid crystal elements, the plurality of unit-electrodes are arranged in substantially the same layout, and the orientation directions of the liquid crystal layers in the two liquid crystal elements are arranged to be substantially orthogonal, further the lens control section outputs different control signals to the two liquid crystal elements, and the two liquid crystal elements are independently controlled.

(20) In the eyeglasses according to (19), it may also be characterized in that when one of the two liquid crystal elements is controlled to have a focal length corresponding to an object at a predetermined distance, the other of the two liquid crystal elements is controlled to have a focal length corresponding to an object at a distance different from the predetermined distance.

(21) In the eyeglasses according to (19), it may also be characterized in that wherein the two liquid crystal elements are a first liquid crystal element and a second liquid crystal element, and when the lens control unit controls the focal lengths of the first liquid crystal element and the second liquid crystal element to have the same or different target values, the second liquid crystal element is controlled to reach the same or different target values with a delay from the first liquid crystal element.

(22) The eyeglasses according to (19), it may also be characterized in that including a gaze information acquisition unit that acquires information regarding the user's gaze, and one of the two liquid crystal elements is controlled based on information acquired by the gaze information acquisition unit, and the other of the two liquid crystal elements is controlled differently from the one based on information acquired by the gaze information acquisition unit.

(23) The present invention is characterized in that the eyeglasses are configured to include a liquid crystal element having a liquid crystal layer and a plurality of unit-electrodes to produce a Fresnel lens-like refractive index distribution in the liquid crystal layer and a lens control unit that outputs a control voltage to the liquid crystal layer in the liquid crystal element, where the liquid crystal element having a plurality of regions having different distances from the optical axis of the refractive index distribution, and the lens control unit is configured to be able to set different focal lengths for each of the plurality of regions.

(24) In the eyeglasses according to (23), it may also be characterized in that wherein the plurality of regions includes at least two regions, and when a target focal length value is set for each of the two regions, the lens control unit sets a target value of focal length for each of the two regions, the lens control unit controls the other of the two regions so that the other reaches the target value later than one of the two regions.

(25) In the eyeglasses according to (23), it may also be characterized in that wherein the plurality of regions includes at least two regions, and when a target focal length value is set for each of the two regions, the lens control unit may control the focal point of one of the two regions, which is located on the side farther from the optical axis, is controlled to vibrate for a predetermined period of time.

(26) In the eyeglasses according to (23), it may also be characterized in that wherein the plurality of regions includes at least two regions, and when a target focal length value is set for each of the two regions, the lens control unit may control the image quality of one of the two regions disposed on the side farther from the optical axis to deteriorate for a predetermined period of time.

(27) In the eyeglasses according to any one of (23) to (26), wherein each of the plurality of regions may be an annular or band-shaped region having a different distance from the optical axis of the refractive index distribution.

(28) In the eyeglasses according to any one of (23) to (27), including a gaze information acquisition unit that acquires information regarding the user's gaze, and the focal length of one of the two regions is controlled based on information acquired by the gaze information acquisition unit, and the focal length of the other of the two regions is controlled in a manner different from that of the one region based on the information acquired by the gaze information acquisition unit.

(29) The eyeglasses according to the present invention include a variable focus lens, a storage unit that stores power model information for changing the refractive index distribution of the variable focus lens according to the user's situation and behavior, and a control unit that controls the variable focus lens based on the power model information, and are characterized in that the eyeglasses further includes a receiving unit that receives the power model information from another of the eyeglasses, and the storage unit is configured to be able to store the power model information from the other eyeglasses.

(30) The eyeglass system according to the present invention includes a plurality of eyeglasses each having a variable focus lens, a storage unit that stores power model information for changing the refractive index distribution of the variable focus lens according to the user's situation and behavior, and a control unit that controls the variable focus lens based on the power model information, and the eyeglass system is characterized in that each of the plurality of eyeglasses further includes a transmitting unit that transmits the power model information, and a receiver unit that receives the power model information from other glasses among the plurality of eyeglasses.

(31) The eyeglass system according to the present invention includes a plurality of eyeglasses each having a variable focus lens, and a control unit of the variable focus lens based on power model information for changing the refractive index distribution of the variable focus lens according to the user's situation and behavior, and configured to be able to transmit and receive the power model information, and a server device. Further, the eyeglass system is characterized in that the server device includes a power model information storage unit for storing the power model information transmitted from the plurality of eyeglasses and associated with user identification information, and the plurality of eyeglasses obtain the power model information associated with the user's identification information from the server device by accepting an instruction from the user.

(32) The eyeglass system according to (31) may be characterized in that the plurality of eyeglasses includes at least two types of eyeglasses of an eyeglass-type information terminal device having a function of displaying images or information superimposed on the real world, and an image display device that covers the wearer's eyes, glasses that do not have the superimposed display function and the image display device.

(33) The eyeglass system according to (31) or (32), may be characterized in that at least some of the plurality of eyeglasses include an update control unit that updates the power model information, and a transmitting unit that transmits the power model information by the update control unit, and the server device stores the updated power model information in the power model information storage unit in association with user identification information.

(34) In the eyeglass system according to (33), may be characterized in that the power model information storage unit stores the updated power model information together with the power model information before the update, further the server device includes a diagnostic information storage unit storing diagnostic information regarding a user's predetermined disease, and a learning data construction unit that constructs a learning data-set including the power model information and the diagnostic information, and a model generation unit that generates a learning model that outputs information related to predicting diagnosis details for the predetermined disease, by machine learning using the power model information included in the learning data-set as input data and the diagnostic information included in the learning data as teacher data, and the input data includes the current power setting information and some previously accumulated power setting information.

(35) The eyeglass system according to (33), may be characterized in that the server device includes a power model information storage unit that accumulates and stores the updated power model information together with the power model information before the update, and a learning data construction unit that constructs a learning data-set including the power model information, and a model generation unit that generates a learning model for outputting information regarding the power model information that is suitable for the current user by machine learning using the power setting information included in the learning data-set as input data and teacher data, and the input data includes a plurality of the power model information, and the teacher data includes the power model information accumulated after the plurality of power model information included in the input data.

(36) The program according to the present invention may be characterized by causing the computer of the server device in the eyeglass system of (33) to accumulate and store the updated power model information in the power model information storage unit together with the power model information before update, to store diagnostic information regarding a user's predetermined disease, to construct a learning data-set including the power model information and the diagnostic information. Furthermore, the program causes a computer to execute a process of generating a learning model that outputs information related to predicting diagnosis details for the predetermined disease, by machine learning using the power model information included in the learning data-set as input data and the diagnostic information included in the learning data as teacher data, and the input data includes the current power setting information and some previously accumulated power setting information.

(37) The program according to the present invention may be characterized by causing the computer of the server device in the eyeglass system of (33) to execute processing for accumulating and storing information and generating the updated power model information in the power model information storage unit together with the power model information before update, and constructing a learning data-set including the power model information, and generating a learning model for outputting information regarding the power model information that is suitable for the current user by machine learning based on the learning data-set. Further, the program may be characterized in that the power model information storage unit stores information indicating update timing in association with the power model information, and input data for machine learning based on the learning data-set includes a plurality of the power model information. Further, the teacher data for machine learning based on the learning data-set includes the power model information accumulated after the plurality of the power model information. Furthermore, the input data includes information indicating the update timing of the power model information accumulated after the above, and information indicating the update timing of at least one of a plurality of the power model information.

(38) The present invention also provides an optical element having a first liquid crystal element and a second liquid crystal element capable of generating a Fresnel type cylindrical lens-like refractive index distribution, wherein the first liquid crystal element generates a linear focal axis in a first direction, and the second liquid crystal element generates a linear focal axis in a second direction, further the first liquid crystal element and the second liquid crystal element are arranged so as to overlap each other so that the first direction and the second direction form an angle with each other avoiding a substantially perpendicular direction.

(39) In the optical element according to (38), it is characterized in that the first liquid crystal element and the second liquid crystal element are arranged such that the first direction and the second direction form a narrow angle of 20 degrees or more and less than 70 degrees.

(40) In the optical element according to (38), may further include a third liquid crystal element capable of generating a refractive index distribution in the shape of a Fresnel type cylindrical lens and generating a focal axis in a third direction, and the three liquid crystal elements are arranged in an overlapping manner, and the first liquid crystal element and the second liquid crystal element are arranged so that the first direction and the second direction form a narrow angle of 50 degrees or more and 70 degrees or less, and the third liquid crystal element may be arranged such that the third direction forms a narrow angle of 50 degrees or more and 70 degrees or less with each of the first direction and the second direction.

(41) In the optical element according to (40) may be characterized in that the first direction, the second direction, and the third direction form a narrow angle of approximately 60 degrees.

(42) In the optical element according to (41), may be characterized in that the amount and direction of astigmatism generated are changed by individually controlling the refractive index distribution of the first to third liquid crystal elements.

(43) In the optical element according to any one of (40) to (42), may further comprising a fourth liquid crystal element capable of generating a concentric Fresnel lens-like refractive index distribution in the liquid crystal layer, and the first to fourth liquid crystal elements have a plurality of unit-electrodes including a first electrode, a second electrode, and a resistive layer having a higher electrical resistivity than the first electrode and the second electrode, and the plurality of unit-electrodes are divided into a plurality of unit-electrode groups having a common input wiring, and each of the plurality of unit-electrode groups constitutes a common input section, further a plurality of common input sections in each of the first to third liquid crystal elements are constituted by the unit-electrode group extending in the direction in which the focal axis is generated, and the plurality of common input sections in the fourth liquid crystal element are arranged at different distances from the optical axis and are constituted by the unit-electrodes group extending in an arc shape.

(44) The glasses may be characterized by being equipped with the optical element of any one of (38) to (43).

(45) The eyeglasses according to the present invention are variable-focus eyeglasses, it may be characterized in that include a variable-focus lens, a lens control unit that controls the refractive index distribution of the variable-focus lens, a gaze information acquisition unit that acquires information regarding the wearer's gaze, and has a power model information storage unit that holds power model information for changing the refractive index distribution of the variable focus lens according to the situation and behavior of the wearer, and the lens control unit determines a refractive index distribution to be set for the variable focus lenses based on the information regarding the gaze and the power model information.

(46) In the eyeglasses according to (45), it may be characterized in that wherein the gaze information acquisition unit includes a gaze space information acquisition unit that acquires information about the state of a space covered by the wearer's gaze, and a gaze behavior detection unit that detects the gaze direction of at least one eye of the wearer, and the lens control unit determines the refractive index distribution to be set for the variable focus lens by deriving a distance to an object of interest of the wearer based on information about the space covered by the gaze and the gaze direction of the at least one eye.

(47) In the eyeglasses according to (45), it may be characterized in that wherein the gaze information acquisition unit includes a gaze behavior detection unit that detects the gaze direction of both eyes of the wearer, and the lens control unit determines the refractive index distribution to be set for the variable focus lens by deriving a distance to an object of interest of the wearer based on information about the space covered by the gaze and the gaze direction of both eyes of the wearer.

(48) In the eyeglasses according to (45), it may be characterized in that wherein the gaze information acquisition unit includes a gaze behavior detection unit that detects a pupil size of at least one eye of the wearer, and the lens control unit determines the refractive index distribution to be set for the variable focus lens by deriving a distance to an object of interest of the wearer based on the pupil size.

(49) In the glasses according to (45), it may be characterized in that include a specific behavior detection unit that detects a specific behavior of the wearer, and the lens control unit change the refractive index distribution of the variable focus lens, when the refractive index distribution of the variable focus lens is set based on the information regarding the gaze and the power model information and when the specific behavior is detected.

(50) in the eyeglasses according to (49), it may be characterized in that wherein the gaze information acquisition unit includes a gaze behavior detection unit that detects gaze behavior by acquiring an eyeball image of the wearer at a first sampling rate, and the specific behavior detection unit detects the specific behavior by acquiring the eyeball image at a second sampling rate higher than the first sampling rate.

(51) In the glasses according to (49) or (50), it may be characterized in that wherein further comprising an update control unit that updates the power model information stored in the power model information storage unit, the update control unit updates the power model information based on the situation and behavior of the wearer and the refractive index distribution of the variable focus lens when the state in which the specific behavior is detected by the specific behavior detection unit ends.

(52) The eyeglasses according to the present invention are variable focus eyeglasses having a variable focus lens, a lens control unit that controls the refractive index distribution of the variable focus lens, and a specific behavior detection unit that detects a specific behavior of the wearer that occurs when the power of the variable focus lens is not suitable. The lens control unit is characterized in that, in a state in which the specific behavior is detected by the specific behavior detection unit, the lens control unit performs control to increase or decrease the power of the variable focus lens so that the specific behavior is reduced.

(53) The eyeglasses according to the present invention are variable focus eyeglasses having a variable focus lens, a lens control unit that controls the refractive index distribution of the variable focus lens, and a specific behavior detection unit that detects a specific behavior of the wearer that occurs when the power of the variable focus lens is not suitable. The lens control unit is characterized in that, in a state in which the specific behavior is detected by the specific behavior detection unit, is performed to cover power within a predetermined range in order to search for an area with a power setting in which the specific behavior does not occur.

(54) The optical element according to the present invention is characterized in that it is an optical element that includes a liquid crystal layer, a plurality of unit-electrodes each having a first electrode and a second electrode and a resistance layer having a higher electrical resistivity than the first electrode and the second electrode and extending linearly with substantially the same width, and a control unit that supplies individual control voltages to each of the first electrodes of the plurality of unit-electrodes and supplies individual control voltages to each of the second electrodes of the plurality of unit-electrodes, thereby generating a linear Fresnel-like refractive index distribution in the liquid crystal layer. Furthermore, the control unit in this optical element is configured to be able to adjust the position of the optical axis in the refractive index distribution of the linear Fresnel lens.

(55) In the optical element according to (54), it may be characterized in that the optical axis in the linear Fresnel-like refractive index distribution is controlled to be located at the boundary between two adjacent unit-electrodes.

(56) The optical element according to (54), it may be characterized in that the control unit controls the optical axis to be located at the center of one of the unit-electrodes, and in the unit-electrode where the optical axis is located, the same control voltage is input to the first electrode and the second electrode.

(57) In the optical element according to (56), it may be characterized in that the control unit controls the optical axis to be located at the center of one of the unit-electrodes when generating the convex linear Fresnel refractive index distribution in the liquid crystal layer. At this time, a retardation value at the center of the unit-electrode where the optical axis is located is higher than retardation values at the first electrode and the second electrode.

(58) In the optical element according to any one of (54) to (57), it may be characterized in that the linear Fresnel lens-like refractive index distribution includes a plurality of retardation gradients having a top and a bottom, and at least some of the plurality of retardation gradients have a retardation value at the top that is lower than an upper limit value of retardation in the liquid crystal layer.

(59) In the optical element according to any one of (54) to (57), it may be characterized in that the linear Fresnel lens-like refractive index distribution includes a plurality of retardation gradients having a top and a bottom, and at least some of the plurality of retardation gradients have a retardation value at the top that is the same as the upper limit value of retardation in the liquid crystal layer.

(60) In the optical element according to any one of (54) to (57), it may be characterized in that the linear Fresnel lens-like refractive index distribution includes a plurality of retardation gradients, and at least a portion of the plurality of retardation gradients, generated by a control voltage higher than a threshold voltage of the liquid crystal layer is input to one of the first electrode and the second electrode on the low potential side.

(61) In the optical element according to any one of (54) to (57), it may be characterized in that the linear Fresnel lens-like refractive index distribution includes a plurality of retardation gradients, and at least a portion of the plurality of retardation gradients, generated by a control voltage lower than a threshold voltage of the liquid crystal layer is input to one of the first electrode and the second electrode on the low potential side.

(62) In the optical element according to any one of (54) to (61), it may be characterized in that the linear Fresnel lens-like refractive index distribution includes a retardation gradient generated across two or more the unit-electrodes.

(63) In the optical element according to any one of (54) to (61), it may be characterized in that the control unit is configured to be able to control the position of the lens part that generates the linear Fresnel lens-like refractive index distribution and the position of a non-lens part that does not have a lens function outside the lens part, and at least a portion of the non-lens portion is controlled to have degraded image quality.

(64) The present invention may include two optical elements of any one of (54) to (57), it may be characterized in that the two optical elements are arranged in an overlapping manner so that the extending direction in which the plurality of unit-electrodes in one of the two optical elements extend substantially perpendicular to the extending direction of the plurality of unit-electrodes in the other of the two optical elements.

(65) The present invention includes three optical elements of any one of (54) to (57), it may be characterized in that the three optical elements are arranged in an overlapping manner, and at this time, each of the extending directions of the plurality of unit-electrodes in the three optical elements may have a narrow angle formed with the other two extending directions of 50 degrees or more and 70 degrees or less.

(66) The present invention may be characterized by providing eyeglasses having any one of 54 to 65 optical elements.

(67) In the eyeglasses according to (66), it may be characterized in that including a gaze information acquisition unit that acquires information regarding the wearer's gaze, and the position of the optical axis of the linear Fresnel lens-shaped refractive index distribution is controlled based on the information regarding the gaze.

(68) The eyeglasses according to the present invention are characterized in that they include having a variable prism element having a liquid crystal layer and a gaze information acquisition unit that acquires information regarding the gaze of the wearer, and further include a control unit that controls the deflection angle of the variable prism element based on information regarding the gaze.

(69) In the glasses according to (68), it may be characterized in that further comprising a storage unit that stores prism control information for controlling the polarization angle according to the wearer's behavior or situation, the control unit controls the deflection angle based on information regarding the gaze and the prism control information.

(70) In the eyeglasses according to any one of (68) to (69), it may be characterized in that further comprising an eye fatigue information acquisition unit that acquires eye fatigue information regarding the degree of eye fatigue of the wearer, and the control unit controls the deflection angle based on information regarding the eye fatigue information.

(71) In the eyeglasses according to any one of (68) to (70), it may be characterized in that further comprising an attention distance derivation unit that derives a distance to an object to which the wearer pays attention, and the control unit controls the deflection angle based on the distance.

(72) In the eyeglasses according to any one of (68) to (70), it may be characterized in that the gaze information acquisition unit is a gaze behavior detection unit that detects the gaze direction of both eyes of the wearer, and the control unit controls the deflection angle based on the direction of gaze of both eyes.

(73) In the eyeglasses according to any one of (68) to (70), it may be characterized in that the gaze information acquisition unit is a gaze behavior detection unit that detects the size of a pupil of at least one eye of the wearer, and the control unit controls the deflection angle based on the size of the pupil.

(74) In the eyeglasses according to any one of (68) to (70), it may be characterized in that the control unit generates the deflection angle when the position of the object to which the wearer pays attention is far from or near based on a predetermined position.

(75) In the eyeglasses according to any one of (68) to (69), it may be characterized in that further comprising means for presuming the position of an object to be gazed at by the wearer, and the gaze information acquisition unit includes a gaze behavior detection unit that detects the gaze direction of both eyes of the wearer, the control unit controls the deflection angle based on a gaze direction to which the wearer should direct both eyes and a gaze direction of both eyes detected based on the gaze direction.

(76) In the eyeglasses of (71), it may be characterized in that the gaze information acquisition unit is a gaze behavior detection unit that detects the gaze direction of both eyes of the wearer, and the prism control information is information indicating the relationship between the distance of the gaze target from the wearer and the convergence angle the wearer's eyes, and the control unit controls the deflection angle based on the convergence angle derived based on the prism control information and the distance, and the convergence angle detected based on the gaze direction of the both eyes.

(77) In the eyeglasses according to any one of (68) to (70), it may be characterized in that the eyeglasses are glasses-type information equipment that include an image display device that covers the area in front of the wearer's eyes and having a variable focus lens, and further the control unit controls the focal length of the variable focus lens and the deflection angle of the liquid crystal prism when the wearer focuses on a position different from the display surface of the image display device.

(78) In the eyeglasses according to any one of (68) to (70), it may be characterized in that the eyeglasses are glasses-type information equipment equipped with an image display device that covers the front of the wearer's eyes, and the image display device displays an image representing the field of view from the wearer's viewpoint based on three-dimensional space information indicating the situation of the three-dimensional space in which the wearer is placed, and the control unit estimates an object to be gazed at by the wearer based on an image representing the field of view and information regarding the field of view, and derives information regarding the position of the object to be gazed at by the wearer based on the three-dimensional spatial information, and controls the deflection angle based on the information regarding the position.

(79) In the eyeglasses according to (1), it may be characterized in that the lens control unit controls the refractive index distribution of the variable focus lens depending on whether or not a predetermined device receives an output indicating an operating state.

(80) The present invention is characterized in that it is a program for causing a computer to function as eyeglasses having a variable focus lens, a storage unit that stores power model information for changing the refractive index distribution of the variable focus lens according to the user's situation and behavior, and a control unit that controls the variable focus lens based on the power model information, further the program for executing means for receiving the power model information from the other eyeglasses and means for storing the power model information from the other eyeglasses.

(81) The present invention is characterized in that it is a predictive diagnosis system that includes a learning model storage unit that stores the learning model was generated by machine learning based on a learning data-set that includes power model information for controlling the variable focus lenses of the eyeglasses according to the user's situation and behavior, and diagnostic information about the user's predetermined disease, and the power model information is associated with information regarding updated timing, and the input data includes the power model information at the present time and some of the power model information updated in the past than the present time.

(82) The present invention is characterized in that it is a power model information prediction system that includes a learning model storage unit that stores the learning model was generated by machine learning based on a learning data-set that includes power model information for controlling the variable focus lenses of the eyeglasses according to the user's situation and behavior, and a prediction unit that outputs information regarding the power model information that can be adapted to the current user by supplying input data to the learning model, and further the power model information is associated with information regarding updated timing, and the input data includes a plurality of pieces of power model information that were updated in the past compared to the current time.

### Effect of the Invention

According to the present invention, it is possible to provide an optical element and eyeglasses that can improve user convenience and quality of life.

### BRIEF DESCRIPTION OF THE DRAWINGS

FIG. 1 is a diagram showing a schematic structure of an eyeglass system according to a first embodiment of the present invention.

FIG. 2 is a diagram showing a schematic structure of variable focus eyeglasses according to the first embodiment.

FIG. 3A is a diagram for explaining the schematic structure of a variable focus lens according to the first embodiment.

FIG. 3B is a diagram for schematically explaining the arrangement of the electrode structure of the liquid crystal element according to the first embodiment.

FIG. 3C is a schematic diagram of a refractive index distribution appearing in a cross section in any direction passing through the center of the liquid crystal element in FIG. 3B.

FIG. 4 is a schematic diagram for explaining a planar configuration of a unit-electrode U1 in the first embodiment.

FIG. 5 is a schematic diagram for explaining the VV cross section in FIG. 4.

FIG. 6 is a diagram showing the state of the VI-VI cross section in FIG. 3B.

FIG. 7 is an explanatory diagram of lead wires connected to unit-electrodes.

FIG. 8A is a diagram for explaining a fan-shaped area to which a common input unit according to the first embodiment belongs.

FIG. 8B is a diagram for explaining a fan-shaped area in Modification 1 of the first embodiment.

FIG. 9 is a diagram for explaining the functional configuration of the eyeglass system of the first embodiment.

FIG. 10 is a diagram for explaining data for constructing learning data recorded in the server device of the first embodiment.

FIG. 11 is a diagram showing a flow for constructing learning data from information collected by the server device of the first embodiment.

FIG. 12 is an explanatory diagram of a predictive diagnosis model in the first embodiment.

FIG. 13 is a diagram for explaining the functional configuration of an eyeglass system in a second embodiment.

FIG. 14 is a diagram illustrating an example of a flow in a lens power setting support mode process.

FIG. 15A is a diagram for explaining the screen view of lens power setting support mode.

FIG. 15B is a diagram for explaining the screen view of lens power setting support mode.

FIG. 16 is a diagram for explaining data for constructing learning data stored in the server device of the second embodiment.

FIG. 17 is a diagram for explaining data for constructing learning data stored in the server device of the third embodiment.

FIG. 18 is an explanatory diagram of a lens power setting prediction model in the third embodiment.

FIG. 19 is a diagram for explaining the functional configuration of an eyeglass system in a fourth embodiment.

FIG. 20 is a diagram for explaining data for constructing learning data stored in the server device of the fourth embodiment.

FIG. 21 is an explanatory diagram of a lens power setting prediction model in the fourth embodiment.

FIG. 22 is a diagram showing a schematic configuration of an eyeglass system in a fifth embodiment.

FIG. 23 is a diagram for explaining the functional configuration of the eyeglass system according to the fifth embodiment.

FIG. 24 is a diagram for explaining an example of power model information.

FIG. 25 is a diagram for explaining the situation in which power model information is updated.

FIG. 26 is a diagram illustrating a flow of lens power control in variable focus eyeglass according to the fifth embodiment.

FIG. 27A is a diagram for explaining a learning model generated by the eyeglass system of the fifth embodiment.

FIG. 27B is a diagram for explaining a learning model generated by the eyeglass system of the fifth embodiment.

FIG. 28 is a diagram for explaining a common input section and an annular region in a liquid crystal element according to modification 3.

FIG. 29 is a diagram for explaining the configuration of a variable focus lens LN according to a fourth modification of the first embodiment.

FIG. 30 is a diagram for explaining the configuration of a variable focus lens according to modification 5.

FIG. 31A is a diagram for explaining a planar layout of unit-electrodes of one liquid crystal element in modification 5.

FIG. 31B is a schematic diagram showing a situation in which unit-electrodes U1 of six liquid crystal elements of modification 5 extend in respective directions.

FIG. 31C is a diagram showing a situation in which five common input sections of one liquid crystal element are arranged in modification 5.

FIG. 32 is a diagram for explaining the schematic configuration of a variable focus lens according to modification 6.

FIG. 33 is a schematic diagram of a planar configuration of a liquid crystal element according to modification 6.

FIG. 34 is a diagram for explaining the functional configuration of variable focus eyeglasses of modification 6.

FIG. 35A is a diagram for explaining the situation in which a linear Fresnel lens-like refractive index distribution is generated in the liquid crystal element of modification 6.

FIG. 35B is a diagram for explaining the situation in which a linear Fresnel lens-like refractive index distribution is generated in the liquid crystal element of modification 6.

FIG. 35C is a diagram for explaining the situation in which a linear Fresnel lens-like refractive index distribution is generated in the liquid crystal element of modification 6.

FIG. 35D is a diagram for explaining the situation in which a linear Fresnel lens-like refractive index distribution is generated in the liquid crystal element of modification 6.

FIG. 35E is a diagram for explaining the situation in which a linear Fresnel lens-like refractive index distribution is generated in the liquid crystal element of modification 6.

FIG. 35F is a diagram for explaining the situation in which a linear Fresnel lens-like refractive index distribution is generated in the liquid crystal element of modification 6.

FIG. 35G is a diagram for explaining the situation in which a linear Fresnel lens-like refractive index distribution is generated in the liquid crystal element of modification 6.

FIG. 36 is a diagram for explaining the member configuration of a lens portion in variable focus eyeglasses of modification 7.

FIG. 37 is a diagram explaining a situation in which problems in binocular vision function are alleviated by controlling the deflection angle in a variable prism.

FIG. 38 is an explanatory diagram of the functional configuration of the variable focus eyeglasses of modification 7.

FIG. 39 is a diagram illustrating a configuration in which an auxiliary electrode is arranged on the center electrode.

### EMBODIMENT OF THE INVENTION

### First Embodiment

### <1. Overview>

The eyeglass system 1 according to the first embodiment of the present invention will be described below.

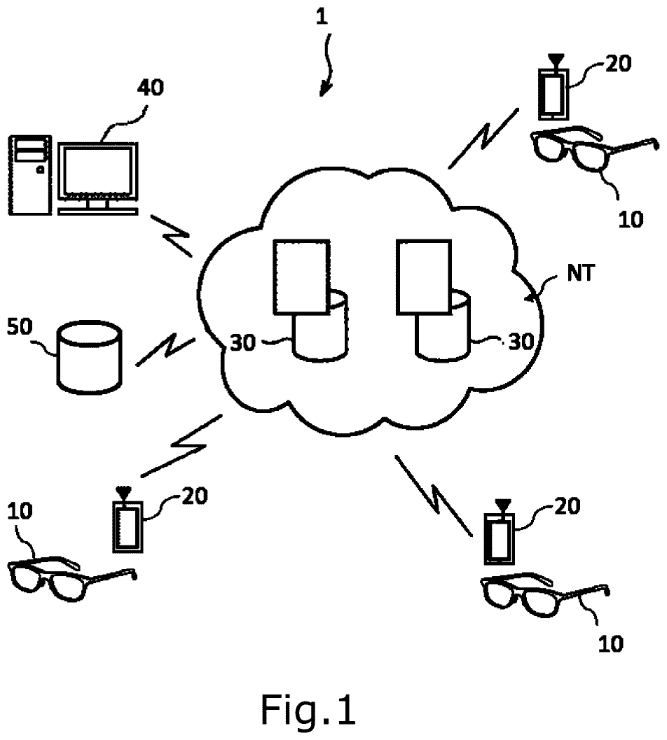

FIG. 1 is a diagram showing a schematic configuration of an eyeglass system 1 according to a first embodiment. As shown in the figure, the eyeglass system 1 of the present embodiment includes variable focus eyeglasses 10 and operation devices 20 that are individually owned by multiple users, a server device 30, a client device 40 for medical workers, and a medical-related database 50. Each operating device 20 and the server device 30 are connected to be able to communicate data with each other via the Internet line NT, and the operation device 20 and the variable focus eyeglasses 10 are connected to be able to communicate data with each other, for example, via a wireless standard such as Bluetooth connection. Further, the medical worker client device 40 and the medical-related database 50 are connected to the server device 30 through the Internet line NT so as to be able to communicate data with each other.

The variable focus eyeglasses 10 of the eyeglass system 1 have power model information for changing a lens power set in the variable focus lenses LN according to the user's situation and behavior. The power model information of the variable focus eyeglasses 10 of this embodiment is configured by associating power setting information with each of the short-range mode (first mode), the medium-range mode (second mode), the long-range mode (third mode). Each mode is switched according to the user's situation, and the power setting information for each mode is arbitrarily input by the user through the operation device 20.

The power setting information held corresponding to each mode of the variable focus eyeglasses 10 is information indicating the contents of the power setting of the variable focus lens LN, and indicates the refractive index distribution set in the variable focus lens LN. The power setting information of this embodiment includes information about three types of setting values indicating the spherical power (SPH), astigmatism axis (AXIS), and degree of astigmatism (CLY) for the left and right lenses. In the variable focus eyeglasses 10, the reference power settings are switched corresponding to each of the first to third modes.

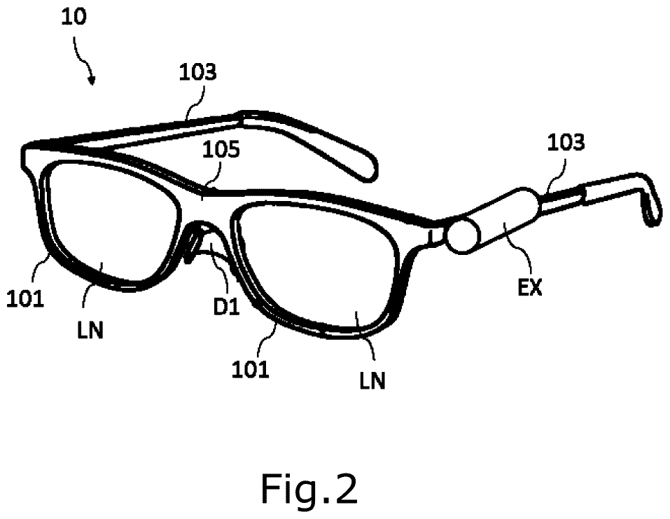

FIG. 2 is a diagram showing a schematic configuration of the variable focus eyeglasses 10. As shown in FIG. 2, the variable focus eyeglasses 10 have a pair of variable focus lenses LN fixed by a pair of rims 101, and further include a pair of temples 103, a bridge 105, and a nose pad D1. Further, an external unit EX is attached to one of the temples 103, and a control circuit, a power supply circuit, and a battery for the variable focus lens LN are built into the rim 101 and the temple 103. The variable focus lens LN is a variable focus liquid crystal lens that can form convex and concave Fresnel lens-like refractive index distributions, the details of which will be described later.

Here, the external unit EX in this embodiment is configured to include a laser ranging module, and is connected to a control circuit built into the temple 103. The laser ranging module is configured to detect objects located on an extension of the temple 103, thereby it is possible to roughly obtain the situation of the space where the gaze of the wearer of the variable focus eyeglasses 10 extends. Further, this external unit EX may have a built-in battery.

Furthermore, the transition between the short-range mode, the medium-range mode, and the long-range mode in the variable focus eyeglasses 10 according to the present embodiment is controlled to switch depending on the distance to the object detected by the laser ranging module. For example, when an object is detected at a distance of less than 60 centimeters, the mode is switched to the short-range mode, and when an object is detected at a distance of 60 centimeters or more and less than 1.5 meters, the mode is switched to the medium-range mode. Then, if an object is not detected at a distance of less than 1.5 meters, it is controlled to switch to the long-range mode.

At least one of the temples 103 and the external unit EX has a built-in communication device and is connected to the operation device 20, so that the user can input the power of the variable focus lens in the first to third modes of the variable focus eyeglasses 10.

Further, the information acquired by the laser ranging module can be acquired as lifestyle information related to the user's lifestyle and living habits, such as the time spent working at a close distance to the eyes.

The external unit EX also has built-in modules such as a 6-axis sensor and illuminance sensor to collect various lifestyle information of the user, such as head tilt habits and outdoor activity time. Various types of information acquired by the variable focus eyeglasses 10 including the external unit EX are transmitted to the server device 30 via the operation device 20. In the server device 30, a learning model is generated by utilizing the information acquired by the variable focus eyeglasses 10 as learning data. The learning model generated by the server device 30 can be used for purposes such as early detection of diseases.

Note that it is preferable that the external unit EX be rotatably attached to the temple 103 of the variable focus eyeglasses 10. Thereby, by rotating, information or an object in a straight line directed downward can be detected using the extension line of the temple 103 as a reference. This is because depending on the user's situation, the transition between the first to third modes may be more comfortable if the state of the space is acquired below (or above) the extension of the temple 103.

### <2. Variable Focus Lens>

Below, the variable focus lens LN included in the variable focus eyeglasses 10 will be explained in detail.

### (2-1) Overview of Variable Focus Lens

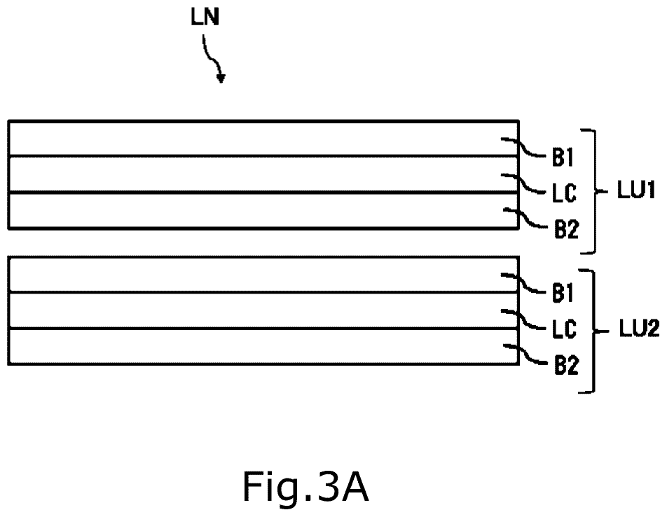

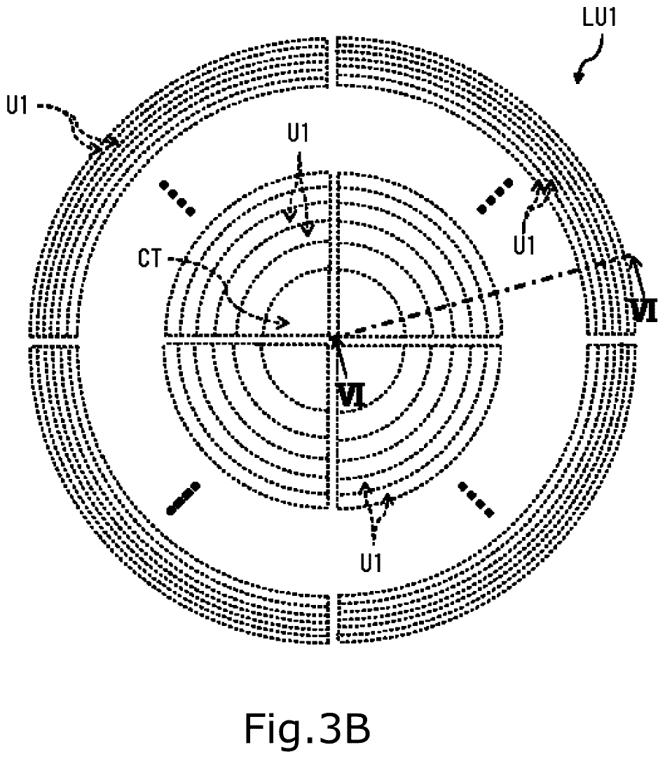

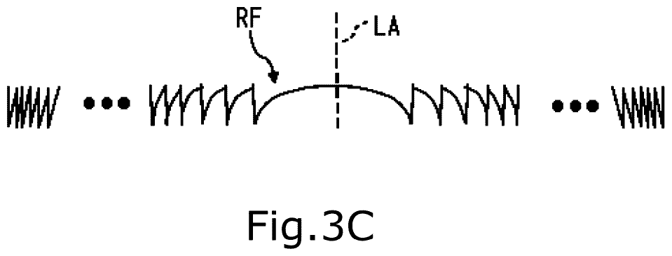

FIG. 3A is a diagram for explaining the schematic configuration of the variable focus lens LN. As shown in FIG. 3A, the variable focus lens LN includes two liquid crystal elements LU1 and LU2, which sandwich a liquid crystal layer LC between a transparent substrate B1 and a transparent substrate B2. The liquid crystal layer LC in the two liquid crystal elements LU1 and LU2 generates a sawtooth refractive index distribution by applying a control voltage, and each function as a Fresnel lens whose focus can be varied.

Further, the two liquid crystal elements LU1 and LU2 have different alignment films in the liquid crystal layer LC so that the alignment directions are perpendicular to each other, but other than this, they have the same configuration. Therefore, in the following description, regarding the structures of the two liquid crystal elements LU1 and LU2, similar structures will be omitted as appropriate. Because the alignment directions of the liquid crystal layers LC in the two liquid crystal elements LU1 and LU2 are perpendicular to each other, it is possible to refract each of the p-polarized light component and the s-polarized light component of the incident light.

FIG. 3B is a diagram for schematically explaining the arrangement of the electrode structure in the liquid crystal element LU1. As shown in the figure, in the liquid crystal element LU1, a center electrode CT is arranged at the center, and a plurality of arc-shaped unit-electrode U1 are arranged concentrically around the center electrode CT. Each unit-electrode U1 of this embodiment corresponds to an annular shape having a central angle of approximately 90 degrees. In other words, they are arranged in succession in the radial direction in each of four fan-shaped regions having a central angle of approximately 90 degrees.

FIG. 3C is a schematic diagram of the refractive index distribution RF appearing in a cross section in any direction passing through the center of the liquid crystal element LU1 in FIG. 3B. In the liquid crystal element LU1, an electric field is applied to the liquid crystal layer LC by voltage being supplied to the center electrode CT and each unit-electrode U1, thereby generating a sawtooth refractive index distribution RF. Further, the refractive index distribution RF is formed to be substantially symmetrical about the optical axis LA, and when viewed from above, the undulations of the refractive index are distributed concentrically. The potential gradient generated at the center electrode CT and each unit-electrode U1 corresponds to each sawtooth undulation in the sawtooth-like refractive index distribution. Therefore, the liquid crystal element LU1 functions as a convex (or concave) Fresnel lens whose focal length can be varied. Note that in this specification, planar view refers to viewing from the direction of the optical axis LA in the liquid crystal element LU1, that is, from the direction perpendicular to the transparent substrate B1.

2-2) about Unit-Electrodes

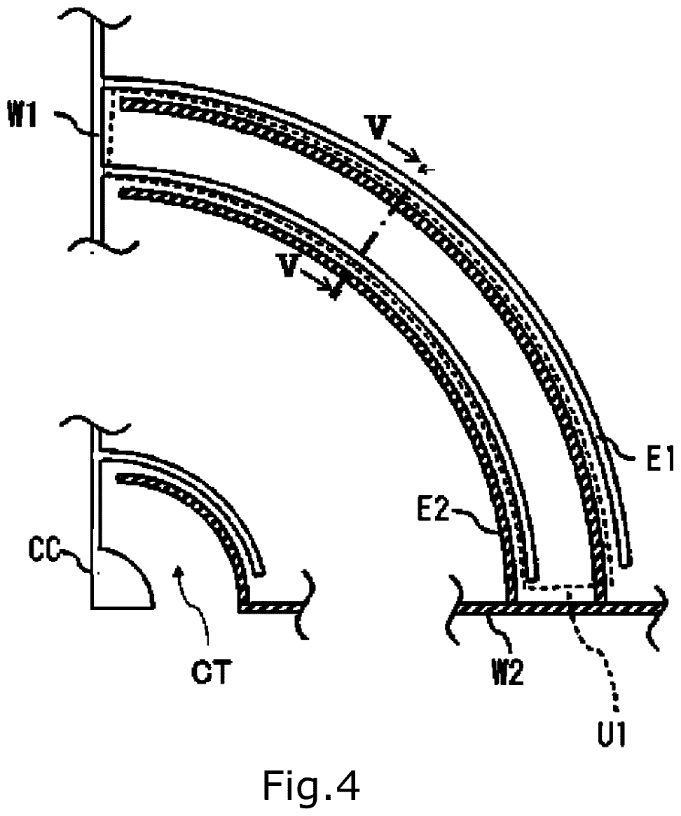

FIG. 4 is a schematic diagram for explaining the planar configuration of one unit-electrode U1. As shown in FIGS. 3B and 4, the unit-electrode U1 of the present embodiment is defined by an arcuate region of approximately 90 degrees, and includes a first electrode E1 and a second electrode E2 formed in a linear shape. Moreover, the unit-electrode U1 has a space formed between the first electrode E1 and the second electrode E2 that is wider than the line width thereof. By applying different voltages to the first electrode E1 and the second electrode E2, a potential gradient can be generated in the space.

Further, the first electrode E1 and the second electrode E2 extend in an arc shape along the outer shape of each unit-electrode U1. Then, it is connected to the first lead wire W1 and the second lead wire W2, respectively: to form a sawtooth shape. In the two unit-electrodes U1 that are adjacent to each other in the radial direction, the first electrode E1 of the unit-electrode U1 arranged on the outer circumference side and the second electrode E2 of the unit electrode U1 arranged on the inner circumference side are disposed adjacent to each other with a narrow space between them.

Note that the first lead wire W1 and the second lead wire W2 extend in the radial direction, and are provided between adjacent unit-electrode U1 in the circumferential direction in FIG. 3B. Further, in the liquid crystal element LU1 according to the present embodiment, there are lead wires other than the first lead wire W1 and the second lead wire W2 in the space between the unit-electrode U1 adjacent to each other in the circumferential direction, which will be described in detail later.

Furthermore, the center electrode CT disposed at the center of the liquid crystal element LU1 has a fan-shaped (or other shape such as a disk shape) core electrode CC instead of the first electrode E1, thereby a potential gradient can be generated in the region between the second electrode E2 connected to the second lead wire W2 and the core electrode CC.

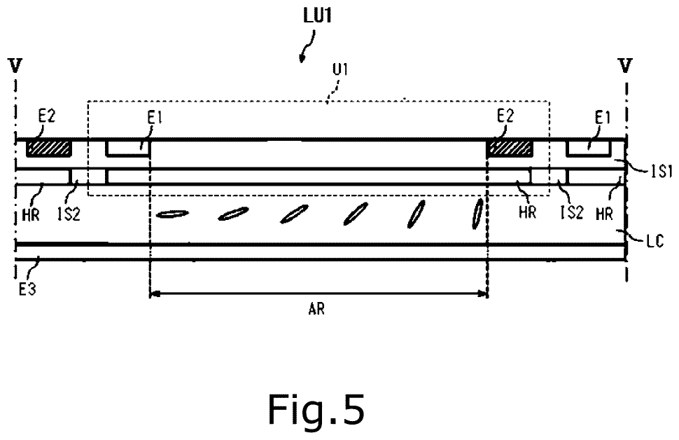

FIG. 5 is a schematic diagram for explaining the VV cross section in FIG. 4, but some components are omitted for simplification. The VV cross section in FIG. 4 is a radial cross section passing through a position corresponding to the center of the concentric arrangement of each unit-electrode U1. In the following, the structure of the unit-electrode U1 will be explained in more detail using FIG. 5, and the potential distribution applied to the liquid crystal layer LC by the unit-electrode U1 and the refractive index distribution caused by the potential distribution will be explained.

The liquid crystal layer LC in FIG. 5 is sandwiched between a substrate located above in the drawing (transparent substrate B1 in FIG. 3A) and a substrate located below in the drawing (transparent substrate B2 in FIG. 3A). The former substrate is constructed by laminating a first electrode E1, a second electrode E2, an insulating layer IS1, a resistance layer HR, and an insulating layer IS2 on a glass substrate (not shown in FIG. 5), and the latter substrate is constructed by laminating a counter electrode E3 on a glass substrate (not shown in FIG. 5).

In the transparent substrate B1, a first electrode E1 and a second electrode E2 are formed on a glass substrate, and an insulating layer IS1 is laminated so as to bury the first electrode E1 and the second electrode E2. Further, a resistive layer HR is laminated on the insulating layer IS1, and an insulating layer IS2 is further arranged to fill in the space between the resistive layers HR. Further, in the transparent substrate B2, a counter electrode E3 is formed on the glass substrate. Further, the transparent substrate B1 and the transparent substrate B2 have anti-parallel parallel alignment films at the interface of the liquid crystal layer LC, but these are not shown for the sake of simplification.

The liquid crystal layer LC is, for example, a nematic liquid crystal, and the orientation of the liquid crystal becomes a homogeneous orientation in an environment without an electric field where no voltage is applied from the first electrode E1 and the second electrode E2, and the color of the liquid crystal is transparent. Further, the thickness of the liquid crystal layer LC in this embodiment is preferably 5 μm or more and 30 μm or less.

The first electrode E1 and the second electrode E2 are formed of a transparent conductive film such as ITO (Indium Tin Oxide). Further, as shown in FIG. 3, the area AR within the unit-electrode U1 is defined by the space between the first electrode E1 and the second electrode E2, and is interposed between the first electrode E1 and the second electrode E2. The width of the region AR is larger than the line widths of the first electrode E1 and the second electrode E2.

The insulating layer IS1 is a transparent electrical insulator, and is formed of silicon dioxide (SiO₂), for example. The insulating layer IS1 in this embodiment is laminated so as to embed structures such as the first electrode E1, the second electrode E2, the first lead wire W1, and the second lead wire W2. Further, the insulating layer IS2 is laminated so as to fill the space between the resistive layers HR formed on the insulating layer IS1. The insulating layer IS2 may be formed by burying the resistive layer HR with silicon dioxide similar to the insulating layer IS1, or may be formed by burying the resistive layer HR with an alignment film extending at the interface of the liquid crystal layer LC.

The resistance layer HR has a larger electrical resistivity than the first electrode E1 and the second electrode E2, and a smaller electrical resistivity than the insulating layer IS1 made of silicon dioxide, and for example it is composed of a transparent film such as zinc oxide (ZnO). The sheet resistivity of the resistance layer HR is higher than each of the sheet resistivity of the first electrode E1 and the sheet resistivity of the second electrode E2, and is smaller than the sheet resistivity of the insulating layer IS1. The sheet resistivity of a material is the value obtained by dividing the electrical resistivity of the material by the thickness of the material. Further, the electrical resistivity of the resistive layer HR is preferably 1 Ω·m or more, and the sheet resistivity of the resistive layer HR is preferably 1×10²Ω/□ or more and 1×10¹¹Ω/□ or less.

Further, the resistance layer HR in this embodiment is configured to be included in the unit-electrode U1, and its planar shape is an arcuate shape with a width slightly narrower than the width of the unit electrode U1. And the resistive layer HR is formed separated from the resistance layer HR in the other adjacent unit-electrode U1. In the resistance layer HR is preferably formed so as to be electrically isolated from the resistance layers HR in the other unit-electrode U1. Further, the resistance layer HR is arranged in a region AR between the first electrode E1 and the second electrode E2 when viewed in a plan view. As shown in FIG. 5, the resistance layer HR extends between the first electrode E1 and the second electrode E2, overlapping at least a portion of the first electrode E1, and furthermore, it is preferable to form it so as to overlap at least a portion of the second electrode E2. However, it is not necessarily limited to such an aspect.

Further, as shown in FIGS. 4 and 5, the first electrode E1 of the unit-electrode U1 is formed along the second electrode E2 of the other adjacent unit-electrode U1, and the second electrode E2 of the unit-electrode U1 is formed along the first electrode E1 of another adjacent unit-electrode U1. Between the first electrode E1 of the unit-electrode U1 and the second electrode E2 of another adjacent unit-electrode U1, and between the second electrode E2 of the unit-electrode U1 and the first electrode E1 of another adjacent unit-electrode U1, form a boundary. An insulating layer IS1 is disposed at this boundary; and the resistance layers HR between adjacent unit-electrodes are separated by an insulating layer IS2.

In this embodiment, as shown in FIGS. 4 and 5, the boundary between unit-electrodes is defined at the center of the insulating layer IS1 interposed between radially adjacent unit-electrodes. Further, in this embodiment, the unit-electrode U1, the first electrode E1, the second electrode E2, and the resistance layer HR included therein are each extended in the circumferential direction of concentric circles and formed in an arc shape. Further, these widths refer to size corresponding to the thickness in the radial direction of concentric circles.

Note that the width of the insulating layer IS1 interposed between two unit-electrode U1 adjacent in the radial direction may be set to, for example, 15 μm or less and 5 μm or more, or may be narrowed depending on the distance from the optical axis LA of the liquid crystal element LU1.

The counter electrode E3 is formed in a planar shape on the transparent substrate B2 using, for example, a transparent conductive film such as ITO. A ground potential (0V) is supplied to the counter electrode E3 in this embodiment.

### (2-3) About Refractive Index Distribution of Unit-Electrode

Next, the potential distribution and refractive index distribution caused in the liquid crystal layer LC by the unit-electrode U1 will be explained using FIG. 5.

First, a first voltage V1 is supplied to the first electrode E1 via the first lead wire W1 based on an input from a control section (not shown) in the liquid crystal element LU1. Similarly, the second voltage V2 is also supplied to the second electrode E2 from the control unit via the second lead wire W2. The first voltage V1 and the second voltage V2 in this embodiment are rectangular wave alternating current voltage and have the same frequency and phase, but the phases and frequencies do not necessarily have to be the same. Further, the voltage does not have to be a rectangular wave alternating current voltage. Further, the maximum amplitude of the first voltage V1 and the second voltage V2 is set to be, for example, 10 V or less, and the frequency is set to, for example, 10 Hz or more and 1 kHz or less.

Here, when the first voltage V1 and the second voltage V2 are different voltages and the second voltage V2 has a higher effective value than the first voltage V1, as shown in FIG. 5, the liquid crystal molecules change from being a state parallel to the transparent substrate B1 to a state standing vertically from the first electrode E1 side (low potential side) to the second electrode E2 side (high potential side). Specifically; a liquid crystal layer LC is interposed between the first electrode E1, the second electrode E2, and the counter electrode E3, and a resistive layer HR is further arranged between the first electrode E1 and the second electrode E2 in planar view. This configuration generates a potential distribution that gradually transitions from the potential of the second voltage V2 of the second electrode E2 to the potential of the first voltage V1 of the first electrode E1. As a result, a refractive index distribution is caused in each unit-electrode U1 in the liquid crystal layer LC. Further, the refractive index gradient caused in the liquid crystal layer LC changes depending on the width of the region AR in the unit-electrode U1, and the gradient tends to become steeper as the width becomes narrower.

### (2-4) Regarding Arrangement of Unit-Electrodes

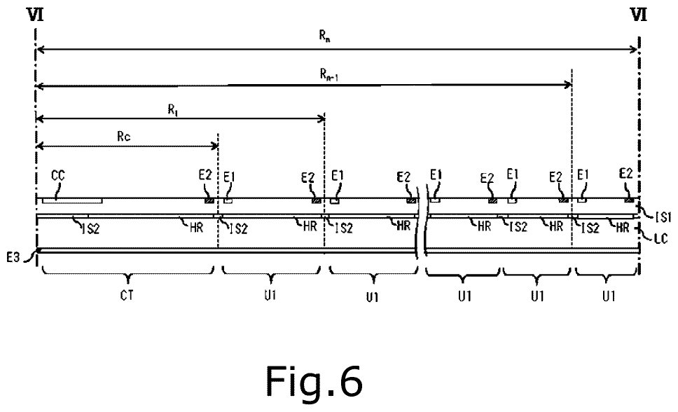

FIG. 6 shows the state of the VI-VI cross section in FIG. 3B, and is a radial cross-sectional view passing near the optical axis of the liquid crystal element LU1. A center electrode CT including a core electrode CC is arranged at the center of the liquid crystal element LU1, and unit-electrode U1 is arranged radially around the center electrode CT. In this specification, the boundary between the center electrode CT and the unit-electrode U1 is defined at the center of the insulating layer IS1 interposed therebetween, and the radius Rc of the center electrode CT corresponds to the distance from the optical axis of the liquid crystal element LU1 (a position corresponding to the center of the concentric arrangement of the unit-electrode U1) to the boundary:

Further, as shown in FIG. 6, the radius of the unit-electrode U1 is assumed to be Rn, and the subscript n is an integer from 1 to N, which is assigned to each of the plurality of unit-electrodes. Further, the subscript n is assigned in ascending order from the unit electrode with the smallest radius to the unit electrode with the largest radius among the plurality of unit electrodes U1. For example, a numerical value such as 50 or 60 may be assigned to N, or a larger numerical value may be assigned to increase the aperture of the variable focus lens LN. Furthermore, as shown in the same figure, the size of the radius of the unit-electrode U1 corresponds to the distance from the optical axis of the liquid crystal element LU1 to the boundary on the outer peripheral side (second electrode E2 side) of the unit-electrode U1.

The radius Rn of the unit-electrode U1 in this embodiment is expressed by the following equation (1).

$$\begin{matrix}[ Math 1 ] & \\ {R}_{n} = {( n + 1 )}^{1 / 2} \times {R}_{c} & ( 1 )\end{matrix}$$

### (2-5) About the Lead Wire

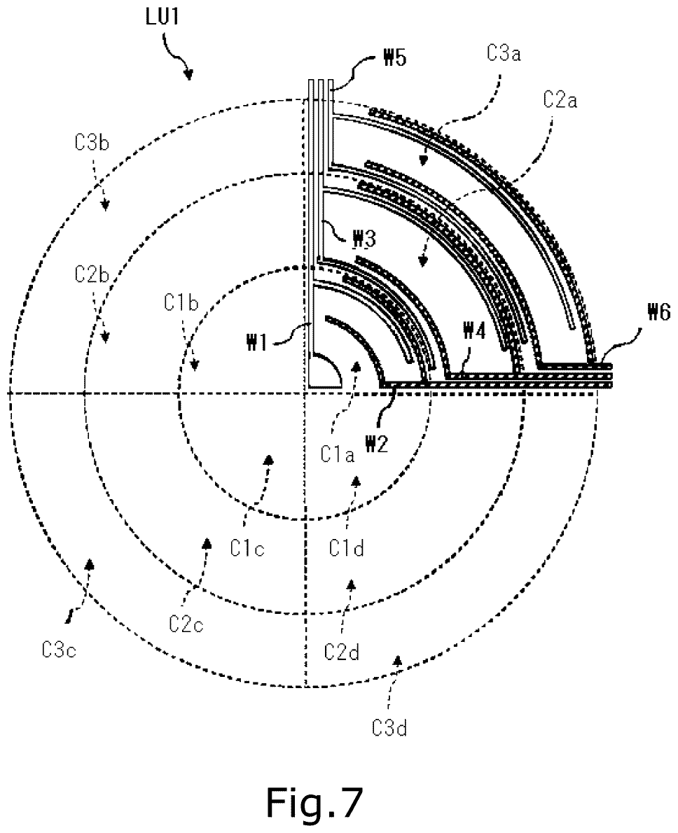

Next, the lead wire connected to the unit-electrode U1 will be explained using FIG. 7. In the liquid crystal element LU1 according to this embodiment, the plurality of unit-electrode U1 are divided into several unit-electrode groups. Furthermore, a plurality of combinations of lead wires for input to the first electrode E1 and lead wires for input to the second electrode E2 are provided so that input to different unit electrode groups can be individually controlled.

As shown in FIG. 7, the liquid crystal element LU1 has a plurality of common input sections C1a to C1d, C2a to C2d, and C3a to C3d each consisting of 12 areas, and each of the common input sections corresponds to an area whose distance from the optical axis LA is within a predetermined range.

Specifically, the common input sections C1a to C1d correspond to four fan-shaped areas with a central angle of approximately 90 degrees, and similarly, the common input sections C2a to C2d, C3a to C3d also corresponds to four annular regions having a center angle of 90 degrees and a thickness in the radial direction. Further, as shown in FIG. 7, the common input sections C1a, C2a, C3a are arranged in order from the optical axis LA of the liquid crystal element LU1, and the same is true for the common input sections C1b, C2b, C3b, etc.

Further, the plurality of unit-electrode U1 in the liquid crystal element LU1 belong to any common input section, and the unit-electrode group of each common input section, therefore the unit-electrode group of each common input section, the input to the first electrode E1 is common, and the input to the second electrode E2 is common.

Further, as shown in FIG. 7, the first lead wire W1 is connected to the first electrode E1 group of the unit-electrode U1 belonging to the common input section C1a, and the second lead wire W2 is connected to the second electrode E2 group. As a result, the first electrode E1 group and the first lead wire W1, and the second electrode E2 group and the second lead wire W2 have a comb-like structure and are arranged so as to be nested with each other. Similarly, a third lead wire W3 is connected to the first electrode E1 group of the unit-electrodes belonging to the common input section C2a, and a fourth lead wire W4 is connected to the second electrode E2 group of the unit-electrodes belonging to the common input section C2a, and a fifth lead wire W5 is connected to the first electrode E1 group of the unit-electrodes belonging to the common input section C3a, and a sixth lead wire W6 is connected to the second electrode E2 group of the unit-electrodes belonging to the common input section C3a.

Although the illustrations in FIG. 7 are omitted as appropriate, the lead wires W1, W3, and W5 extend toward the center where the core electrode CC is arranged from the 180 degrees direction, 270 degrees direction, and 0 degrees direction in the same figure. The lead wires W2, W4, and W6 also extend toward the center where the core electrode CC is arranged from the 90s degrees direction, the 180 degrees direction, and the 270 degrees direction in the same figure. Also, in the common input sections C1b to C1d. C2b to C2d. C3b to C3d, the first electrode E1 group of the unit-electrode and the lead wires W1, W3, W5 are connected as in the case of the common input sections C1a, C2a, C3a, and the lead wires W2, W4, and W6 are connected to the second electrode E2 group of the unit-electrode.

A control signal from a control unit in the variable focus eyeglasses 10 is input to each of the plurality of common input sections C1a to C1d, C2a to C2d, and C3a to C3d in this embodiment via connected lead wires. In the liquid crystal element LU1, in order to prevent astigmatism as described below from occurring, the four common input portions adjacent in the circumferential direction and forming an annular shape are uniformly controlled. Therefore, three types of control signals are input to 12 common input sections.

### (2-6) About Eye Astigmatism Correction

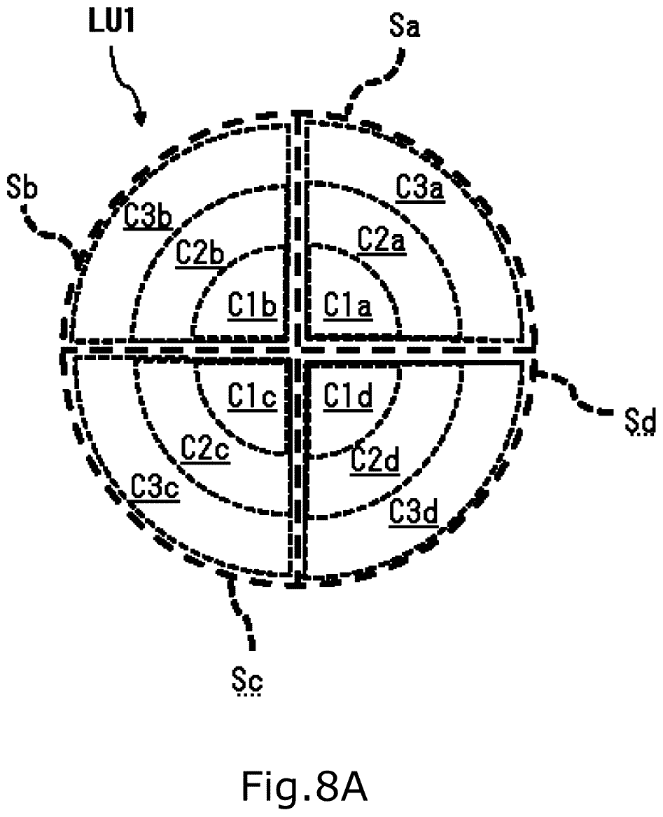

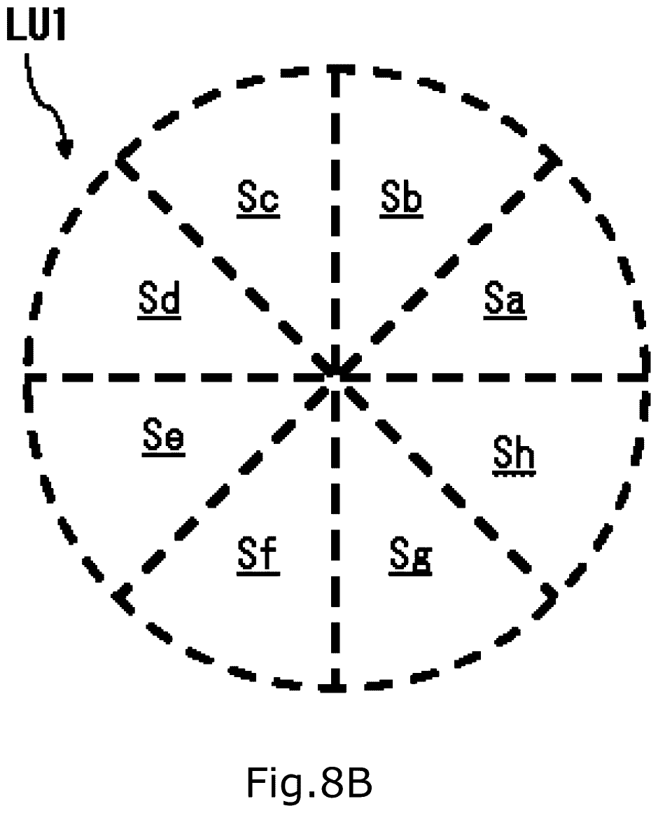

Further, FIG. 8A is a diagram for explaining fan-shaped areas Sa to Sd to which the common input sections C1a to C1d, C2a to C2d, and C3a to C3d belong. As shown in FIGS. 7 and 8A, the liquid crystal element LU1 is divided into four fan-shaped areas having a center angle of 90 degrees by lead wires (not shown), and the common input sections C1a to C3a are belongs to the first fan-shaped area Sa, the common input sections C1b to C3b belong to the second fan-shaped area Sb, the common input parts C1c to C3c belong to the third fan-shaped area Sc, and the common input parts C1d to C3d belong to the fourth fan-shaped area Sd.

The liquid crystal element LU1 is divided into fan-shaped area having a predetermined central angle and an even number of 4 or more. Then, control is performed so that fan-shaped area facing each other via the optical axis LA (fan-shaped area disposed at symmetrical positions with the optical axis LA as the center) have the same focal length. At the same time, astigmatism can be generated by controlling the focal length to be different in any fan-shaped area that does not have the above relationship. In other words, two fan-shaped areas having a symmetrical positional relationship with respect to the optical axis LA form one set, and the liquid crystal element controls the focal length of each set. More specifically; for example, a set of fan-shaped areas (first pair) is set to a first focal length, and a set of fan-shaped areas (first pair) in a direction rotated 90 degrees with respect to the set of fan-shaped areas (a second pair) is set to a second focal length different from the first focal length. Then, when there is one or more sets of fan-shaped areas (one or more pairs) arranged between the first pair and the second pair, the focal length of the one or more pairs is set to gradually change from the first focal length to the second focal length. (The focal length is changed from the first focal length to closer to the second focal length as the second pair is approached). The astigmatism is generated by the above settings. As a result, even if the user of the variable focus eyeglasses 10 has eye astigmatism, a visual field with astigmatism-corrected power is provided.

Further, FIG. 8B is a diagram for explaining a liquid crystal element LU1 according to modification example 1 of the present embodiment. The liquid crystal element LU1 of this modification is divided into eight fan-shaped regions having a central angle of 45 degrees. Lead wires extending toward the optical axis LA extend in eight directions, and control signals are input to the unit-electrode U1 belonging to each fan-shaped area. In the first modification, four types of focal lengths are set by four sets of fan-shaped areas facing each other via the optical axis LA. This is preferable because it is possible to generate astigmatism corresponding to eight types of astigmatic axes. (Two sets of fan-shaped areas Sa, Sh, Sd, and Se facing each other via the optical axis LA are set to have the same focal length. At this time, by setting the focal lengths of the remaining two fan-shaped areas to a focal length that is farther than the same focal length, it is possible to correspond to the astigmatism axis in the 0 degrees direction (horizontal direction in FIG. 8B). Furthermore, one set of fan-shaped areas Sa, Se is set to the same focal length, and two sets of fan-shaped areas Sb, Sf and fan-shaped areas Sh, Sd are set to the same focal length. Further, a pair of fan-shaped areas Sc, Sg are set to have the same focal length. With such settings, it is possible to accommodate an astigmatic axis in the 22.5 degrees direction.)

### <3. About the Eyeglass System>

Next, a description will be given of an eyeglass system 1 including variable focus eyeglasses 10 configured with the above-mentioned variable focus lens LN.

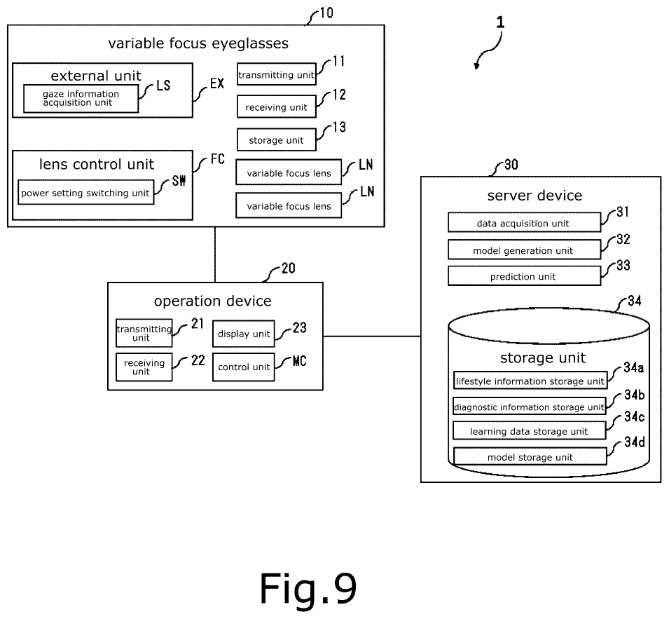

FIG. 9 is a diagram for explaining the functional configuration of the eyeglass system 1. The variable focus eyeglasses 10, the operation device 20, and the server device 30 are realized by including storage element such as RAM (Random Access Memory) and ROM (Read Only Memory), a storage area constituted by hard disks, SSDs (Solid State Drives) etc., and a program-controlled device such as a CPU (Central Processing Unit). Further, in the variable focus eyeglasses 10, the operation device 20, and the server device 30, each function is realized by the CPU executing a program stored in a storage area such as a hard disk.

As shown in FIG. 9, the variable focus eyeglasses 10 consists of including two variable focus lenses LN, a lens control unit FC, an external unit EX, a transmitting unit 11, a receiving unit 12, and a storage unit 13. Further, the operation device 20 consists of a transmitting unit 21, a receiving unit 22, a display unit 23, and a control unit MC. The server device 30 also consists of a data acquisition unit 31, a model generation unit 32, a prediction unit 33, and a storage unit 34.

### (3-1) Functional Configuration of Variable Focus Eyeglasses

The lens control unit FC in the variable focus eyeglasses 10 has a power setting switching unit SW, and the external unit EX has a gaze information acquisition unit LS. In the storage unit 13 of this embodiment holds power model information configured by associating three types of power setting information with each of the first to third modes. The power setting switching unit SW refers to the power setting information held in the storage unit 13 based on the information acquired by the gaze information acquisition unit LS, and performs control to change the power of the variable focus lenses LN for both the left and right eyes.

The gaze information acquisition unit LS is configured to include a sensor that acquires information regarding the user's gaze. The gaze information acquisition unit LS in this embodiment is a gaze space information acquisition unit that acquires information about the state of the space covered by the user's gaze, and as described above, is configured by a laser ranging module. The laser ranging module can detect an object in a space on an extension of the temple 103 when the variable focus eyeglasses 10 are used, and constantly outputs distance information to the detected object.

Furthermore, the gaze space information acquisition unit is not limited to a distance sensor such as a laser ranging module, but may be an image sensor, a depth camera capable of acquiring three-dimensional information, or a LiDAR (Light Detection and ranging) sensor may also be used. In other words, it is not limited to this, as long as information regarding the state of the space covered by the gaze of the user wearing the variable focus glasses 10 can be obtained. In addition, in the case of an image sensor, a real-time object detection algorithm such as YOLO (You Look Only Once) is implemented in the lens control unit FC, and when a specific object (for example, a mobile phone or an open book) is detected with a bounding box of a certain size or more, the power setting switching unit SW may switch the power setting of the variable focus glasses 10. Further, in the case of a depth camera or a LiDAR sensor, the power setting switching unit SW may switch the power setting of the variable focus glasses 10 according to the acquired three-dimensional information.

Further, the gaze information acquisition unit LS may include a gaze behavior detection unit that acquires information regarding the user's gaze behavior as gaze information. As this gaze behavior detection unit, for example, two or three electro-oculography sensors may be provided on the nose pad D1, or a plurality of electro-oculography sensors may be provided on the temple part of the temple 103 on the wearer's side. However, a sensor that detects the behavior of the user's eyes at the upper (or lower, etc.) of the pair of rims 101 may be used.

The electrodes included in the electro-oculography sensor come into contact with the skin of the wearer to detect the electro-oculogram at the center portions of the left and right eyes and the temples, thereby detecting the movement of the wearer's eyes. Further, the sensors arranged on the pair of rims 101 for detecting the behavior of the eyes may include, for example, a light source such as an LED (Light Emitting Diode) and an imaging unit such as a camera. As the eye tracking technology for acquiring gaze behavior information regarding the gaze behavior of the user of the variable focus eyeglasses 10, for example, a non-contact type such as a corneal reflex method, a dark pupil method, or a bright pupil method may be adopted, or a contact type method such as an electro-oculography method may be adopted, or both a non-contact type and a contact type may be adopted, and the method is not limited to these. The gaze behavior detection unit can be acquired information about eye behavior and eye movements, and collects information such as the number of blinks per day and statistics such as the number and frequency of convergence and divergence movements, and information such as the degree of eye tension.

There is the information regarding the user's gaze acquired by the gaze information acquisition unit LS (gaze related information) and the information indicating the control content of the lens control unit FC based on the information acquired by the gaze information acquisition unit LS (control content information) At least one of them is received by the operation device 20 via the transmitter 11. The information transmitted from the transmitter 11 is not particularly limited as long as it is information acquired by the variable focus glasses 10, such as gaze related information and control content information. For example, the information may be compressed information obtained from a laser ranging module, or may be information regarding the time during which at least one of a plurality of modes is being executed. Furthermore, the information may be information regarding the frequency and history of switching the power setting with the power setting switching unit SW, or may be information acquired by a module such as an illuminance sensor or a 6-axis sensor built into the external unit EX.

### (3-2) Functional Configuration of Operation Device

In this embodiment, the operation device 20 is a mobile terminal such as a smartphone or a tablet owned by the user, and has an application installed thereon for operating the variable focus eyeglasses 10. Note that the operation device 20 may be a wristwatch-type mobile terminal with an application installed thereon for operating the variable focus eyeglasses 10, a stationary PC (Personal Computer), or a dedicated terminal for operating the variable focus eyeglasses 10.

The control unit MC causes the display unit 23 to display and control a screen for inputting power settings in the first to third modes of the variable focus eyeglasses 10. Further, the control unit MC receives a power setting input from the user via the display unit 23 (or by referring to the screen displayed on the display unit 23). The transmitter 21 transmits the received power setting to the variable focus eyeglasses 10 as power setting information.

The receiving unit 22 receives information transmitted from the variable focus eyeglasses 10 and the server device 30. The transmitter 21 also transmits information received from the variable focus eyeglasses 10 to the server device 30.

### (3-3) Functional Configuration of Server Device

The server device 30 is provided, for example, on a cloud.

The data acquisition unit 31 in the server device 30 acquires information transmitted from the variable focus eyeglasses 10 and the like. Specifically, the data acquisition unit 31 in this embodiment includes a lifestyle information acquisition unit, a diagnostic information acquisition unit, a biological information acquisition unit, an attribute information acquisition unit, and a learning data acquisition unit and is constructed. (These are not shown in FIG. 9).

The lifestyle information acquisition unit in the data acquisition unit 31 acquires information related to the user's lifestyle, including information acquired by the variable focus eyeglasses 10, via the network, and stores it in the storage unit 34. The information acquired by the variable focus eyeglasses 10 includes the above-mentioned gaze related information and control content information. Further, the diagnostic information acquisition unit acquires diagnostic information regarding the diagnosis result of a specific disease for the user, for example, by transmitting it from the medical worker client device 40 or the medical-related database 50, and stores it in the storage unit 34. Furthermore, the biometric information acquisition unit and the attribute information acquisition unit acquire biometric information and attribute information by receiving them via the network NT, and store them in the storage unit 34. Details of lifestyle information, etc. will be discussed when explaining the learning data.

In addition, the learning data acquisition unit (learning data construction unit) in the data acquisition unit 31 acquires various information included in the learning data by associating lifestyle information, diagnostic information, etc. with the same user ID. Thereby, the learning data acquisition unit constructs a learning data-set and stores it in the storage unit 34. The model generation unit 32 uses the learning data-set acquired in this manner to generate a predictive diagnostic model that is a learning model that predicts the onset of a specific disease.

The storage unit 34 is composed of a lifestyle information storage unit 34a, a diagnostic information storage unit 34b, a biological information storage unit, an attribute information storage unit, a learning data storage unit 34c, and a model storage unit 34d. A X and Y. As described above, various types of information acquired by the data acquisition section 31 are sequentially accumulated in the storage section 34. Furthermore, the model storage unit 34d stores the predictive diagnostic model generated by the model generation unit 32. A predictive diagnostic model is generated for each type of disease and stored in the model storage unit 34d.

The prediction unit 33 uses the predictive diagnostic model stored in the model generation unit 32 to output a prediction result regarding the onset of a specific disease. The prediction unit 33 may output a prediction result in response to an inquiry from the operation device 20 (or periodically).

The eyeglass system 1 according to the present embodiment described above provides convenience to the user and creates a motivation for using the variable focus eyeglasses 10. Therefore, by using the variable focus eyeglasses 10, it is possible to efficiently collect data such as gaze-related information useful for generating a learning model for predicting the onset of diseases such as dementia and eye diseases that are desired to be detected early. Furthermore, since the server device 30 has a predictive diagnostic model for predicting the onset of a disease, the user of the eyeglass system 1 can receive prediction results of diseases for which early detection is desired.

### <4. About the Learning Data-Set>

In the following, a learning data-set (hereinafter also simply referred to as learning data) acquired by the data acquisition unit 31 of the server device 30 will be explained.

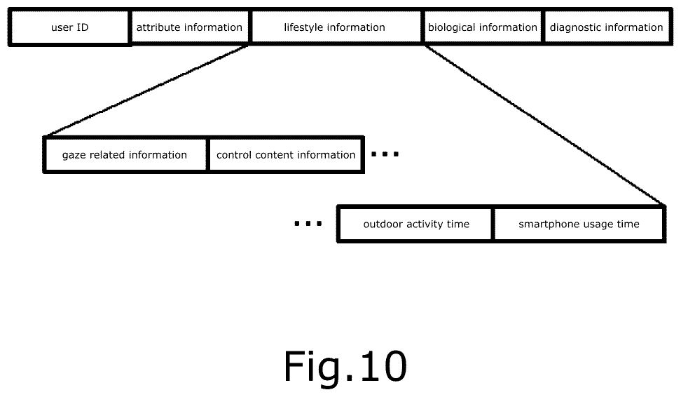

FIG. 10 is a diagram for explaining data for constructing a learning data-set recorded in the storage unit 34c of the server device 30. As shown in the figure, the data for constructing the learning data is data in which a user ID, attribute information, lifestyle information, biological information, and diagnostic information are associated with each other. Regarding the data in the figure, the diagnostic information at the right end of the figure corresponds to “teacher data.” and the portion other than the “teacher data” and user ID corresponds to “input data” when generating a model by machine learning.

The user ID is information for identifying the user of the eyeglass system 1, and is identification information uniquely assigned to the user. The lifestyle information and the like acquired by the data acquisition unit 31 of the server device 30 are associated with a user ID in advance, transmitted from the variable focus eyeglasses 10, etc., and stored in the storage unit 34.

Attribute information is information regarding the individual user, and includes, for example, the user's gender and date of birth (age), but may also include information such as occupation and hobbies.

Lifestyle information is information related to lifestyle and living habits, and in other words, any information that can be used to infer a part of the lifestyle is sufficient. Further, the information may be information for inferring everyday behavior or habits.

As shown in FIG. 10, lifestyle information includes a variety of information. In addition to gaze related information and control content information, for example, information on “outdoor activity time” acquired from an illuminance sensor mounted on the external unit EX of the variable focus glasses 10 can be included. Further, information such as “smartphone usage time” corresponding to the usage time of the user's smartphone, which also serves as the operation device 20, can be included. The lifestyle information is transmitted periodically (or each time it is acquired) from the variable focus eyeglasses 10 and the like, and is stored in the storage unit 34 of the server device 30.

The exposure time of the eyes to the screens of smartphones, tablets, etc. is attracting attention as one of the reasons for the increase in myopia cases, so it is considered useful to include it in the learning data. The learning data may include, for example, lifestyle information such as “time spent in blue light cut display mode” and “time spent using a specific application”. In addition, it may include lifestyle information such as “posture when using a smartphone” and “average distance between the user and smartphone when using a smartphone” is obtained by analyzing the smartphone's built-in front camera and 6-axis sensor.

Furthermore, the lifestyle information is not limited to that acquired from the variable focus eyeglasses 10 or the operation device 20, but also be acquired the server device 30 by being transmitted from other smart devices or PC (personal computer). Therefore. “the usage time of a smart device or a PC (personal computer) other than the operation device 20” may be acquired as lifestyle information. In addition, information such as location information obtained from GPS sensors. WiFi usage information, usage history of specific services, sleeping hours, exercise history obtained by analysis of GPS sensors and 6-axis sensors. etc. may also be acquired as lifestyle information. Furthermore, the lifestyle information may be acquired from an external medical database 50, etc., and may include, for example, information such as “results of a lifestyle questionnaire conducted at a health checkup, etc.”.

Furthermore, the gaze related information included in the lifestyle information may be, for example, information about “the time when an object was detected at a close distance” in the case of a laser distance measurement module. The control content information may be, for example, information indicating the usage time of each of the first to third modes, or information indicating “switching frequency between the first to third modes.” In addition, by analyzing the 6-axis sensor built into the variable focus eyeglasses 10, the server device 30 may acquire indexed information regarding “habit of moving the head and neck” and “posture” as lifestyle information.

In addition, as lifestyle information regarding such content such as time, frequency, and number of times, statistics such as average time and number of times per week or day: may be treated as data for constructing learning data. In addition, statistics such as standard deviations may also be treated as data for constructing training data, and information indicating time-series changes in these statistics may also be treated as information for constructing leaning data. Furthermore, the lifestyle information acquired as time, number of times, etc. as described above may be converted into information indicating a level such as “a lot” or “a little” and may be included as data for constructing learning data. Alternatively, the information may be converted into information indicating a category and included in the data constituting the learning data.

Next, biological information is various physiological/anatomical information such as height, weight, blood pressure, body temperature, etc., and is obtained from a wearable sensor, for example. The biological information may be a statistic such as an average value of time-series changes in measured values. The information may also include information indicating the contents of the user's health checkup and past medical history. The information indicating the contents of this medical checkup may be obtained from a medical database 50 or the like belonging to an external public institution or service provider.

The diagnostic information is information indicating the details of diagnosis by a hospital or the like, and may include information such as the contents of a paper medical record or an electronic medical record at a hospital or clinic. This diagnostic information serves as “teacher data” in the learning data, and may be information indicating the level of progression of a specific disease. Further, the information may be information indicated by labels of 0 and 1, such as “dementia or not” or may be information indicating a category: Furthermore, the diagnostic information may be provided at any time, for example, from the medical worker client device 40 or the like.

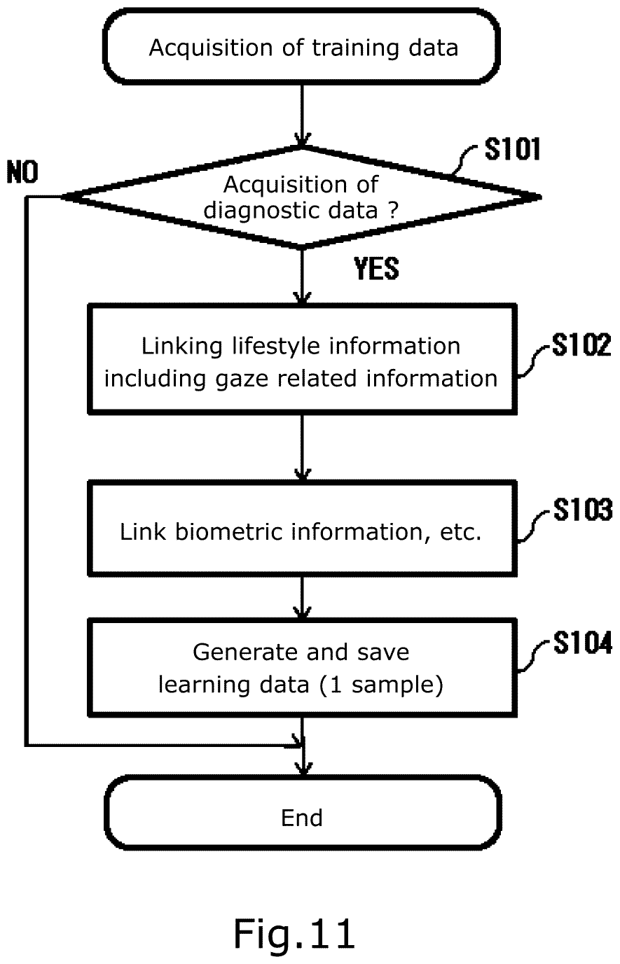

FIG. 11 is a diagram showing a flow of constructing learning data by associating various information collected by the server device 30 with a user ID.

First, in S101, when diagnostic information is transmitted from the medical worker client device 40 or the like to the server device 30 (in the case of YES), the process proceeds to the next S102, and the diagnostic information and lifestyle information are associated. A user ID is assigned to the diagnostic information and lifestyle information, and the data acquisition unit 31 searches the storage unit 34 for lifestyle information having the same user ID as the diagnostic information, and associates this information with the user ID.

Next, in S103, similarly to S102, biological information and attribute information having a common user ID are searched from the storage unit 32, and the searched information is further associated with the diagnostic information and lifestyle information associated in S102. Then, in S104, acquire one sample of learning data based on the various types of associated information and store in the storage unit 32. In S104, generate a multidimensional feature amount based on various types of information associated with the user ID and may store in the storage unit 32. As this multidimensional feature quantity; information that can contribute to effective prediction is selected from various types of information as a feature quantity. Alternatively; it is derived by, for example, generating a new feature amount by combining several pieces of information.

As explained in FIG. 11, the learning data is constructed by; when diagnostic information is acquired, searching and associating lifestyle information and the like with the same user ID as the diagnostic information. Therefore, the eyeglass system 1 is designed to generate learning data by associating accumulated gaze related information and the like by collecting diagnostic information about the user.

Note that in S102, for example, in the case of a user who has just started using the eyeglass system 1 and has not accumulated much information regarding the line of sight in the storage unit 34, steps S102 to S104 may be omitted in order to improve the accuracy of the learning model. That is, the process may end without generating learning data.

### <5. About the Learning Model>

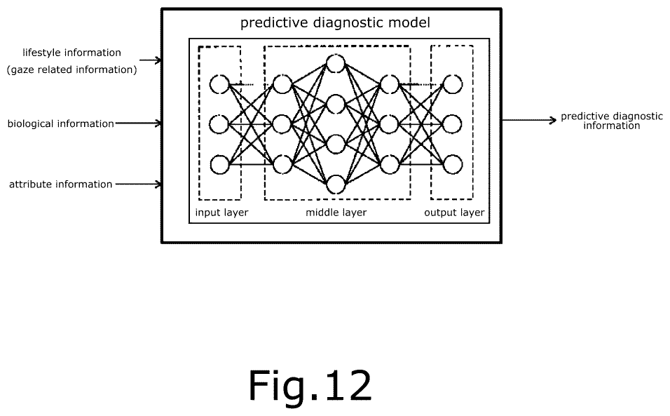

FIG. 12 is a diagram for explaining a predictive diagnosis model that is a learning model in this embodiment. The learning model performs a predetermined calculation on an input value and outputs the calculation result, and the storage unit 34 stores data such as coefficients and threshold values of functions that define this calculation as the learning model. The learning model according to this embodiment is a learned model that has been machine learned in advance. This machine learning is performed so as to output a prediction result when the user diagnoses a predetermined disease in response to input data such as the user's attribute information, lifestyle information, and biological information. Furthermore, the learning model of this embodiment is generated separately and stored in the storage unit 34 for each type of disease that has led to dementia or eye disease.

As shown in FIG. 12, the learning model of this embodiment has a neural network structure in which a plurality of neurons is interconnected. Since this is an existing technology, the details will be omitted, but a neuron is an element that performs calculations on a plurality of inputs and outputs one value as the calculation result. The neuron has information such as weighting coefficients and threshold values used in arithmetic processing. A learning model using a neural network has an input layer, a middle layer, and an output layer. The input layer receives input of one or more data. The intermediate layer performs arithmetic processing on the data received at the input layer. Then, the output layer aggregates the arithmetic results of the intermediate layer and outputs one or more values. The learning process of deep learning is a process of setting appropriate values for the coefficients, thresholds, etc. of each neuron constituting a neural network, using a large amount of learning data given in advance. The learning model of this embodiment is a trained model obtained by performing deep learning on a neural network learning model using teacher data. Note that learning is performed by: for example, gradient descent, stochastic gradient descent, error backpropagation, or the like.

Here, the eyeglass system 1 is a predictive diagnosis system in which a user can receive predictive diagnostic information regarding a specific disease by inquiring the prediction unit 33 of the server device 30.

Specifically: as shown in FIG. 12, the prediction unit 33 first searches the storage unit 34 for the user's “life information.” “biological information.” and “attribute information” in response to an inquiry from the user. As a result, the prediction unit 33 obtains input data to the predictive diagnosis model. Then, the prediction unit 33 executes a process of converting this information into a multidimensional feature corresponding to a learning model and an arithmetic process using a predictive diagnostic model. The predictive diagnostic information obtained as the output is then transmitted to the operation device 20. The contents of the predictive diagnostic information are displayed on the display unit 23 of the operation device 20, thereby allowing the user to obtain predictive diagnostic information regarding a specific disease.

Note that the specific disease targeted for predictive diagnosis by the predictive diagnostic model in the eyeglass system 1 is preferably a disease in which symptoms related to gaze or vision may occur. For example, eye diseases such as cataracts and neurological diseases such as dementia are assumed, but are not particularly limited. A predictive diagnostic model constructed by incorporating information acquired by the variable focus eyeglasses 10 into learning data can contribute to early detection of specific diseases and symptoms.

### Second Embodiment

### <1. Overview>

Next, a description will be given of a spectacle system 1 according to a second embodiment of the present invention.

First, in the eyeglass system 1 according to the second embodiment, the power setting support mode can be executed in the operation device 20. The power setting support mode is a function that allows the user to easily input the power setting in at least one of the first to third modes while adjusting it by himself/herself. The mode is a mode (update support mode) that accepts input from the user for updating the power model information held in the storage unit 13.

Regarding the power setting of the variable focus glasses 10, it is necessary to input six parameters for each mode, similarly to the first embodiment. These six parameters are set by “spherical power (SPH)”. “astigmatism axis (AXIS)”, and “astigmatism degree (CLY)” for both the left and right eyes. For example, if the user wants to adjust the power setting in the first mode (short distance mode), it is assumed that the user adjusts as follows. First, while covering the right eye variable focus lens LN with one hand, operate the operation device 20 with the other hand. It is assumed that this operation involves adjusting the power setting of the variable focus lens LN for the left eye while gazing at a specific subject at a short distance.

Users who set the power in this way are likely to want to focus on finding an appropriate power setting and it is thought that not take their eyes off objects at close or long distances. Furthermore, since there are many combinations of parameters for setting the frequency; it is thought that the user would like to make a decision through trial and error.

From the above, in the power setting support mode of the eyeglass system 1 according to the second embodiment, the user selects in advance one parameter for power setting, and the parameter is switched in stages via the operation device 20. It can be operated. This allows the selected parameter to be increased or decreased in stages, and suitable values for parameters such as SPH, AXIS, CLY, etc. can be comfortably searched for.

Furthermore, in the eyeglass system 1 of the second embodiment, the power setting information input by the user is also transmitted to the server device 30, and is also utilized for constructing learning data for generating a predictive diagnostic model. The eyeglass system 1 of the second embodiment is almost the same as the first embodiment except for the points described above, and the description of the points that are almost the same as the first embodiment will be omitted as appropriate. The second embodiment will be described in further detail below.

### <2. Functional Configuration of Eyeglass System 1>

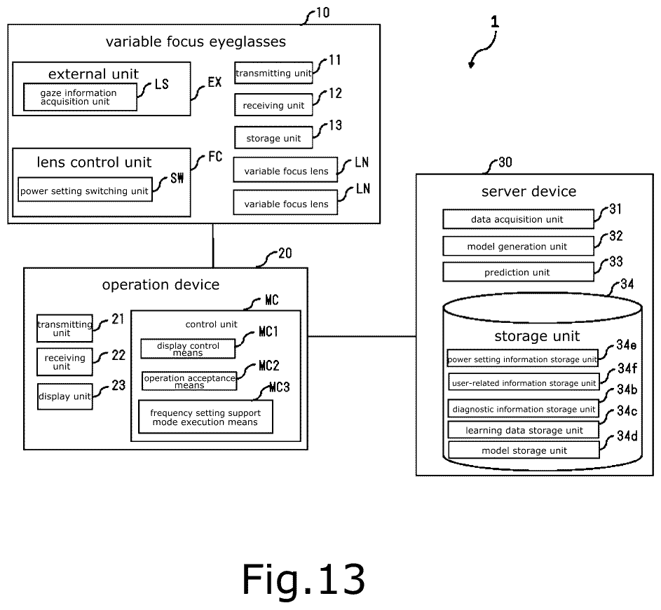

FIG. 13 is a diagram showing the functional configuration of the eyeglass system 1 of the second embodiment. In the operation device 20 of the second embodiment, the control section MC includes a display control means MC1, an operation acceptance means MC2, and a lens power setting support mode execution means MC3.

Furthermore, in the operation device 20 of the second embodiment, the display section 23 is a touch panel that accepts touch operations by the user. The display control means MC1 displays images and the like on the display section 23, and the operation acceptance means MC2 accepts operations input by the user via the display section 23. The control unit MC executes control according to the operation accepted by the operation acceptance means MC2.

The power setting support mode execution means MC3 executes the power setting support mode when the input for executing the power setting support mode is accepted by the operation accepting means MC2. Specifically, first, the control unit MC outputs an instruction to stop the processing by the power setting switching unit SW in the variable focus eyeglasses 10 while the power setting support mode is being executed. The control unit MC controls the value of the selected parameter so that it can be adjusted by increasing (or decreasing) in stages according to the user's input. More specific control by this power setting support mode execution means MC3 will be described later.

When the user inputs the power setting in the power setting support mode, the update control section (not shown) of the variable focus eyeglasses 10 controls the storage unit 13 to overwrite and store the power setting information. Further, this power setting information, information regarding the mode corresponding to the power setting information, and information indicating the date and time when the power setting information was updated are transmitted to the server device 30 by the transmitting unit 21. The data acquisition unit 31 of the server device 30 acquires the power setting information and the like from the operation device 20 and records it in the power setting information storage unit 34e. Furthermore, lifestyle information, biological information, and attribute information are acquired by the data acquisition unit 31 as in the first embodiment and are recorded in the user-related information storage unit 34f.

### <3. About the Power Setting Support Mode>

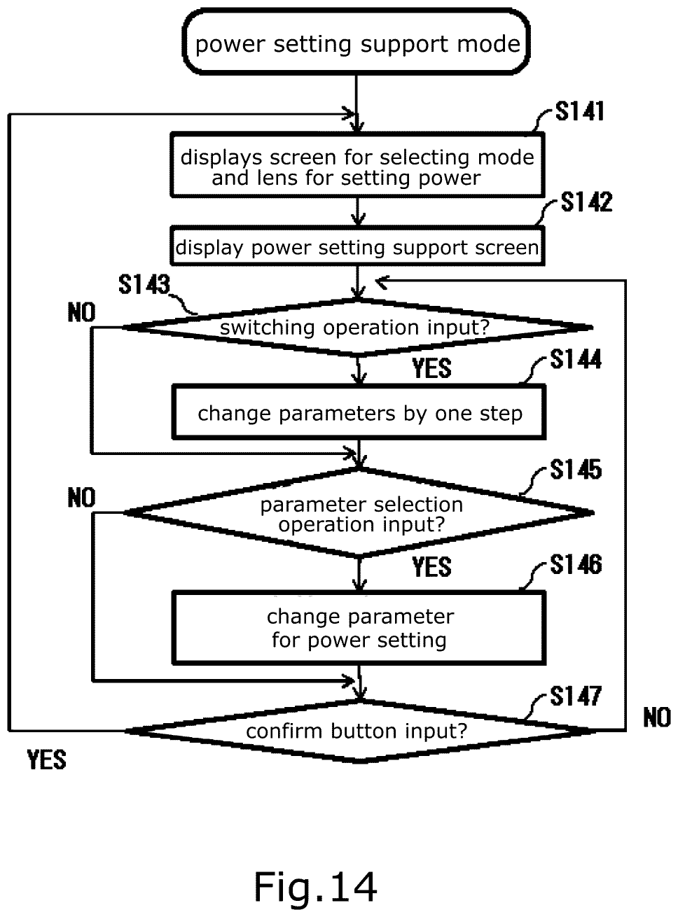

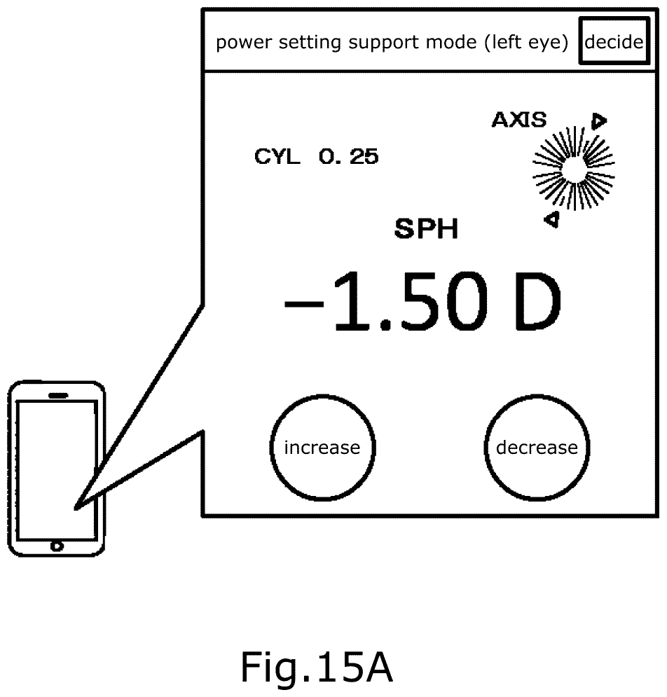

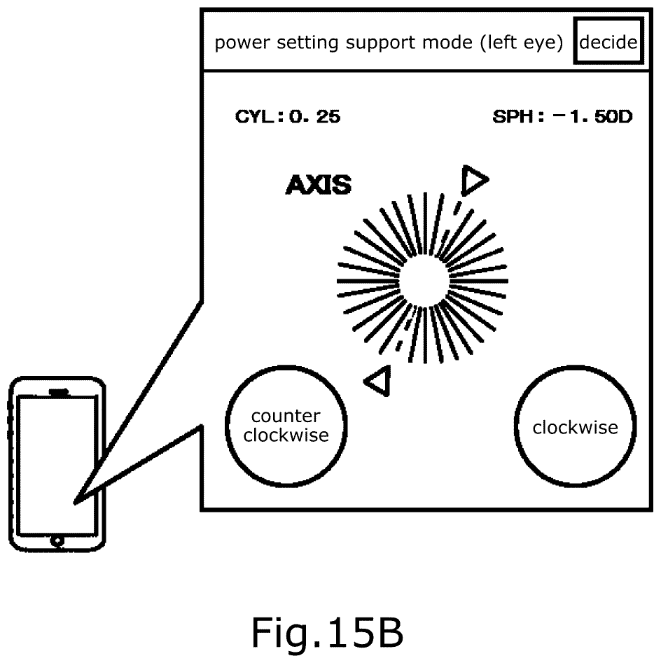

FIG. 14 is a diagram illustrating an example of the flow of processing in the power setting support mode. Moreover, FIGS. 15A and 15B are diagrams for explaining the appearance of the screen in the power setting support mode executed in the operation device 20 of the second embodiment.

As shown in FIG. 14, first in process S141, the display control means MC1 sequentially displays two types of screens. One of the two types of screens is a screen that determines which of the first to third modes the power setting is to be performed, and the other screen is a screen that determines which power setting of the left or right variable focus lens. When the user makes these inputs, the process 142 makes the display unit 23 to display a power setting support mode screen as shown in FIG. 15A.(S142)

On the frequency setting support mode screen, when a switching operation such as “increase” or “decrease” is input (in the case of YES in S143), and when a parameter selection operation is input (in the case of YES in S145), executes the processes of S144 and S146, respectively, and if these processes are not performed, the state where the frequency setting support mode screen is displayed is maintained and the state is in a state of waiting for input from the user (if NO in S143. S145. S147). Furthermore, if the determination button is input (in the case of YES in S147), the process returns to S141, and the process again displays a screen etc. for determining whether or not to set the power of the left and right variable focus lenses LN, and waits for that input by the user.

Note that in case the user wants to end the power setting, a button for ending the power setting support mode may be displayed on the screen displayed in S141. When the user ends the power setting support mode, the power setting information held in the storage unit 13 of the variable focus eyeglasses 10 is updated to the power setting information determined in the power setting support mode. That is, the power model information held in the variable focus eyeglasses 10 is updated. Then, the power setting information is transmitted from the transmitter 21 of the operation device 20 to the server device 30. Further, when transmitting the power setting information to the server device 30, information regarding the corresponding mode of the power setting information and information on the update date and time are transmitted in association with each other.

Next, the display screen in the power setting support mode will be explained using FIGS. 15A and 15B. The power setting support mode screen in FIG. 15A is a display screen when “left eye lens” is selected in S141. As shown in the figure, the names and values of parameters subject to the power setting corresponding to the variable focus lens LN for the left eye are displayed. Furthermore, in the center of the screen, the parameter currently selected to accept stepwise value changes is displayed larger than other parameters. In the illustrated example, the spherical power (SPH) is displayed in a large size, and only that value can be changed. Furthermore, when the user presses the “increase button” or “decrease button” once displayed at the bottom of the figure (YES in S143), the SPH is increased by one step and displayed. At the same time, in conjunction with this operation, the magnification setting of the left eye variable focus lens LN is controlled to change by one step. (S144). Further, when the operation receiving means MC2 receives an operation of pressing near the parameter displayed at the center of the screen (or a swipe operation at the center of the screen), the screen changes as shown in FIG. 15B. As a result, the parameters that can be increased or decreased in stages are controlled to be switched. (S145, S146).

In the above description, it is assumed that the value of the spherical power (SPH) changes by one step when the user presses the “increase button” or “decrease button” once in FIG. 15A. However, this one step may be “±0.25D” or “±0.05D”. Furthermore, it may be possible to perform stepwise adjustment in units smaller than the standard for general eyeglasses.

Further, FIG. 15B shows a state in which the astigmatic axis (AXIS) is switched to an adjustable state, and an image including a figure indicating the direction of the astigmatic axis is displayed. When the user presses the “clockwise button” or “counterclockwise button” at the bottom of the diagram, the tilt angle of the astigmatism axis increases or decreases by a predetermined angle. Specifically; when the operation receiving means MC2 receives an operation of pressing the “clockwise rotation button”, the display control means MC3 displays an image in which a figure indicating the direction of the astigmatism axis is rotated one step clockwise. Further, the control unit MC outputs a command to change the astigmatic axis of the left eye variable focus lens LN, and the lens control unit FC controls the astigmatism axis of the left eye variable focus lens LN to rotate by one step. Note that in FIG. 15B, the figure indicating the direction of the astigmatic axis is composed of two triangles and one broken line, but the figure indicating the direction of the astigmatic axis may be one straight line or a broken line. That is, the shape is not particularly limited as long as the direction of the astigmatic axis can be read.

Since the state of the astigmatic axis is displayed on the operation device 20, the user who sets the power of the variable focus glasses 10 using the power setting support mode can efficiently set the power while comparing the object that the user is gazing at. In addition, the astigmatism power (CYL) takes a positive value in principle, it may be possible to change it to both positive and negative values in the power setting support mode.

In addition, in FIG. 15A, for example, the display control unit MC3 may be cause the display unit 22 to display an evaluation button such as a “GOOD button” for causing the operation acceptance means MC2 to accept a positive evaluation by the user. Furthermore, when the user presses this evaluation button, the value of the spherical power (SPH) may be determined, and the parameter for power setting adjusted by the user may be switched.

### <4. About Learning Data and Learning Models>

Next, the learning data acquired by the data acquisition unit 31 of the server device 30 of the second embodiment and the learning model generated by the model generation unit 32 will be explained.

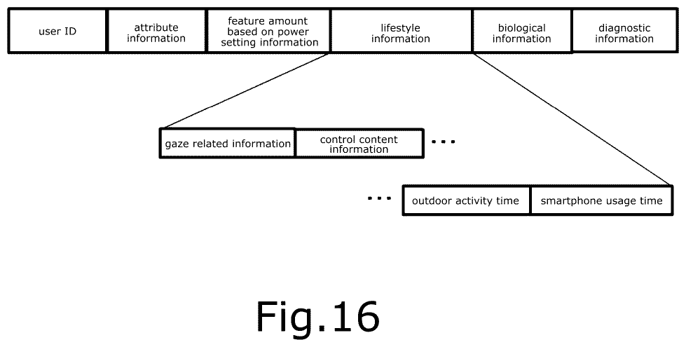

FIG. 16 is a diagram for explaining data for constructing a learning data-set stored in the learning data storage unit 34c in the second embodiment. The data constituting the learning data-set of the second embodiment includes attribute information, lifestyle information, biological information, and diagnostic information associated with a user ID. Furthermore, this embodiment differs from the first embodiment in that power setting information is associated with the user ID.

The learning data in the second embodiment uses diagnostic information regarding a specific disease as teacher data, as in the first embodiment. The data for constructing learning data as shown in FIG. 16 is acquired when diagnostic information is acquired. Specifically, various types of information having the same user ID as the diagnostic information are acquired from the user-related information storage unit 34f and power setting information storage unit 34e of the storage unit 34 and are associated with each other. The input data in the learning data-set is selected or combined from various types of associated information as shown in FIG. 16 to generate multidimensional feature quantities, which are used for machine learning when generating a learning model. The input data in the learning data-set of the second embodiment includes a plurality of pieces of power setting information that have been accumulated in the power setting information storage unit 34e until the diagnostic information that is the teacher data is acquired. However, in FIG. 16, in order to simplify the notation, “feature amount based on power setting information” is used. By including time-series changes in the power setting information in the learning data, the accuracy of predictive diagnosis by the learning model is improved. Note that the plurality of frequency setting information included in the input data may be associated with information regarding “the elapsed time from the update date and time of the power setting until the diagnostic information is acquired” and “the corresponding mode of the power setting information”. These may also be included in the input data.

### Third Embodiment

### <1. Overview>

Next, an eyeglass system 1 according to a third embodiment of the present invention will be described.

The eyeglass system 1 in the third embodiment differs from the second embodiment in the learning data acquired by the data acquisition unit 31 of the server device 30, the learning model generated by the model generation unit 32, and the like. The third embodiment is substantially the same as the second embodiment except for the points mentioned above, and descriptions of the substantially similar points will be omitted as appropriate.

### <2. About Learning Data and Learning Models>

In the eyeglass system 1 according to the third embodiment, three learning models corresponding to each of the first to third modes are generated, and each learning model estimates power setting information suitable for the user in the corresponding mode.

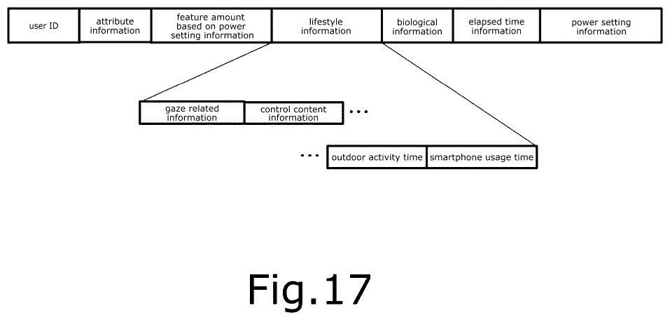

FIG. 17 is a diagram for explaining a data for constructing a learning data-set stored in the learning data storage unit 34c of the server device 30 of the third embodiment. As shown in the figure, the data for constructing the learning data-set in the third embodiment includes “feature value based on power setting information”. “elapsed time information”, and power setting information that serves as teacher data. This differs from the data for constructing the learning data-set of the second embodiment in that it does not include diagnostic information.

The input data in the learning data-set of the third embodiment includes two or more pieces of power setting information that were input before the power setting information serving as teacher data was input. However, in FIG. 17, for simplification of notation, it is written as “feature value based on power setting information”. In the power setting information storage unit 34e of the third embodiment, power setting information is stored in association with information indicating the update date and time, and information regarding the corresponding mode, as in the case of the second embodiment. “Elapsed time information” is information based on information indicating the update date and time of power setting information, which is teacher data, and information indicating the update date and time of the latest power setting information among multiple power setting information included in the input data. As a result, the “elapsed time information” becomes information indicating the length of the period between the above two pieces of information.

The data acquisition unit 31 (learning data acquisition unit, not shown) of the server device 30 acquires various types of information having a common user ID from the storage unit 34 to construct learning data. Then, this construction operation is performed, for example, when frequency setting information is transmitted from the operation device 20, using the frequency setting information as teacher data. Further, the learning data acquisition unit may generate multidimensional feature value from the input data of the learning data-set, store them in the learning data storage unit 34c in advance, and make them available for use during machine learning.

The input data to the learning data of the third embodiment is power setting information corresponding to the same mode as the power setting information serving as the teacher data. Specifically, two or more pieces of power setting information including power setting information having an update date and time closest to the update date and time of the power setting information of the teacher data are used. Note that information on content corresponding to the above-mentioned “elapsed period information” may be acquired as the lifestyle information and biometric information acquired from the user related information storage section 34f in order to construct the learning data.

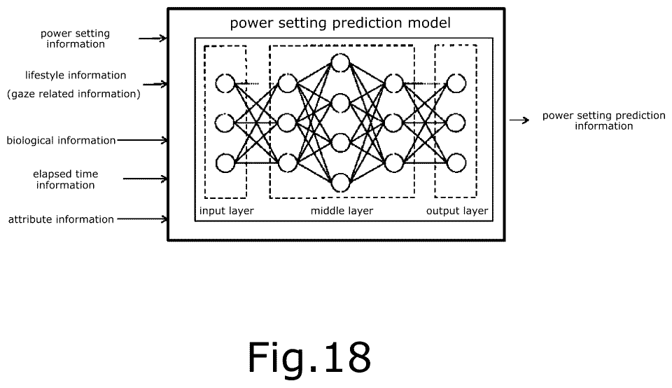

FIG. 18 is a diagram for explaining a configuration example of a learning model in the third embodiment. The input data when predicting predict the frequency setting using the learning model according to the third embodiment include multiple pieces of power setting information previously input by the user, lifestyle information, biological information, clapsed time information, and attribute information. Here, the above-mentioned plurality of power setting information is power setting information that has been previously input and set in the current variable focus eyeglasses 10, and some power setting information that has been further input previously. The learning model (frequency setting prediction model) of the third embodiment is a learned learning model that has undergone machine learning in advance. Therefore, when the above input data is given, the information regarding power setting (power setting prediction information) suitable for the user is output.

In the eyeglass system 1 of the third embodiment, the prediction unit 33 generates input data to three learning models in response to an inquiry from the operation device 20 by the user. Specifically, necessary information required for input to the three learning models is acquired. Note that the above-mentioned necessary information is the power setting information currently set in each mode, some power setting information previously set in each mode, lifestyle information, biological information, elapsed time information, and attribute information. Further, the prediction unit 33 inputs the three input-data to the three learning models to obtain three outputs of power setting prediction information, and the server device 30 transmits the power setting prediction information to the operation device 20. Note that the inquiry from the operation device 20 may be for obtaining power setting prediction information for any of the first to third modes.

The power setting prediction information may be accepted as is in the operation device 20 and the variable focus glasses 10 and updated as power setting information in the storage unit 13 of the variable focus glasses 10, or may be simply displayed on the operation device 20. For example, at the start of execution of the power setting support mode, settings based on the power setting prediction information may be reflected so that the user can try it out.

Note that as the teacher data in the learning data-set, for example, information regarding a difference value or change between power setting information may be used. In this case, for example, the input data in the learning data-set includes a plurality of power setting information, and the teacher data includes power setting information updated after the plurality of power setting information. Furthermore, the power setting information updated at the latest timing among the plurality of power setting information is also included. Then, the power setting prediction model may be generated by machine learning using the difference value of these two pieces of power setting information and the information on the amount of change. Further, in the third embodiment, as shown in FIG. 17, elapsed time information is included as data for constructing learning data, but the present invention is not necessarily limited to this aspect. For example, power setting information can be transmitted from the operation device 20 so as to be recorded in the power setting information storage unit 34c at regular timings, thereby making it possible not to include “elapsed time information.”

Note that the learning data-set of the third embodiment uses information regarding the difference value and change of the power setting information as described above as the teacher data, and at the same time does not include the power setting information in the input data. It is also conceivable that the input data thus obtained could be used to generate multidimensional feature quantities that are utilized during machine learning to generate a learning model. In this case, the prediction unit 33 generates input data to the learning model in response to an inquiry from a user (or periodically). The generation of input data by the prediction unit 33 is performed by collecting information (life information, biological information, elapsed time information, attribute information) required for input to the learning model from the storage unit 34, and this may allow the output from the learning model to be obtained.

Note that the output from the learning model in this case is, for example, information such as a difference value between the power setting currently set by the user and the power setting newly set by the user. That is, this difference value may be used as power setting prediction information. Alternatively: based on whether or not this difference value exceeds a predetermined standard, information indicating whether or not the currently set power setting should be changed (whether or not it is compatible) may be output as power setting prediction information. It is assumed that the input data of the learning data in the third embodiment includes a plurality of pieces of frequency setting information. However, the input data may include at least one frequency setting information and other lifestyle information, and thereby: the accuracy of the frequency setting prediction model can be improved.

### Fourth Embodiment

### <1. Overview>

Next, a description will be given of an eyeglass system 1 according to a fourth embodiment of the present invention. The eyeglass system 1 in the fourth embodiment differs from the third embodiment mainly in the learning data acquired by the data acquisition unit 31 of the server device 30, the learning model generated by the model generation unit 32, and the like. Hereinafter, points that are different from the third embodiment will be mainly described, and descriptions of points that are substantially the same as the third embodiment will be omitted as appropriate.

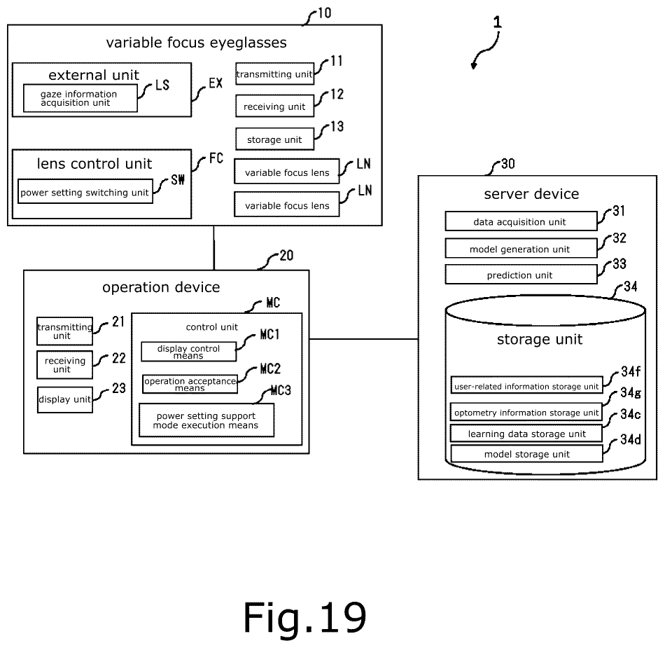

FIG. 19 is a diagram for explaining the functional configuration of the eyeglass system 1 in the fourth embodiment. As shown in the figure, the storage unit 34 of the eyeglass system 1 in the fourth embodiment is configured to be include a user-related information storage unit 34f, an optometry information storage unit 34g, a learning data storage unit 34c, and a model storage unit 34d.

The data acquisition unit 31 acquires optometry information from the external medical database 50 and the medical worker client device 40, and stores it in the optometry information storage unit 34g. This optometry information is information indicating the results of optometry performed at an ophthalmology clinic or the like to manufacture eyeglasses, and information indicating the contents of the ophthalmology prescription at that time. Further, the optometry information only needs to include at least part of the information necessary for manufacturing the glasses, and may be information measured using a simple inspection device or information regarding the distance to the gaze point. Furthermore, the optometry information may be information acquired from an optometrist built into the head-mounted display. Further, similarly to the third embodiment, the data acquisition unit 31 acquires the user's lifestyle information, biological information, and attribute information from the medical-related database 50 and the like, and stores them in the user-related information storage unit 34f.

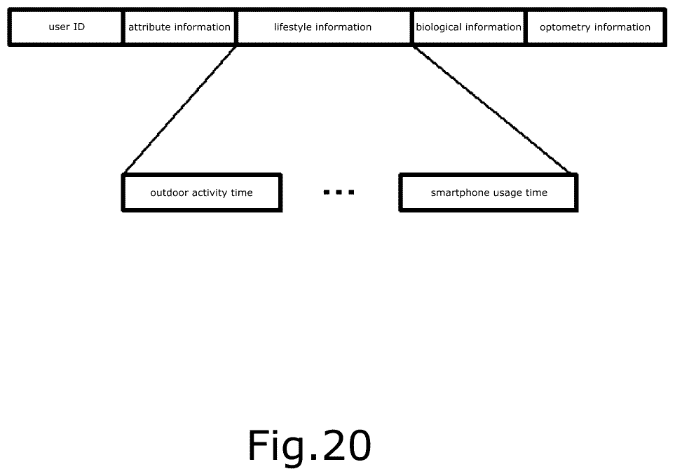

FIG. 20 is a diagram for explaining data for constructing a learning data-set stored in the learning data storage unit 34c of the server device 30 of the fourth embodiment. As shown in the figure, the input data for constructing the learning data-set of the fourth embodiment includes lifestyle information such as “outdoor activity time” and “smartphone usage time” that can be obtained from sources other than the variable focus eyeglasses 10, and configured to include biological information, attribute information, and optometry information, but is not particularly limited to this aspect, and may also include “gaze related information” etc., acquired from the variable focus eyeglasses 10. The “teacher data” in the learning data-set of the fourth embodiment includes optometry information. Further, the learning data storage unit 34c further holds and accumulates multidimensional feature amounts generated by selecting or combining various information in the input data, and are used in machine learning by the model generation unit 32.

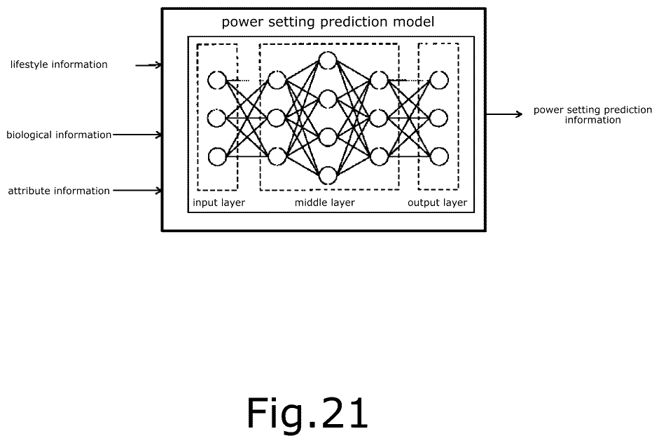

FIG. 21 is a diagram for explaining a frequency setting prediction model in the fourth embodiment. As shown in the figure, when lifestyle information and the like of a user to be predicted is input to the learning model, it is converted into information including multidimensional feature amounts corresponding to the learning model. Then, information regarding the power setting of the variable focus eyeglasses 10 that can be suitable for the prediction target user is output as power setting prediction information. Note that the teacher data in the power setting prediction information or learning data that is the output of the power setting prediction model may be content corresponding to some parameters in the power setting information of the variable focus eyeglasses 10. Note that the optometry information in the fourth embodiment may be information associated with information indicating the date and time of acquisition. Further, as in the case of the third embodiment, the “input data” in the learning data-set may include some pieces of optometry information acquired before the optometry information serving as the teacher data. In addition, by making the input data include two or more pieces of optometry information that were acquired before the optometry information that serves as the teacher data, and the learning data may include time-series changes, thereby improving the accuracy of the power setting information. Further, as in the case of the third embodiment, information regarding difference values and changes between optometry information may be used as the teacher data.

Note that the power setting prediction model in the third embodiment described above may also be a model corresponding to some parameters in the power setting information. Furthermore, in the third embodiment and the like, a learning model may be generated for each parameter of power setting information. The “astigmatism power” and “astigmatism axis” that are parameters of power setting information tend to change depending on age, gender, etc., so by building a learning model for each parameter, the prediction accuracy of power setting information can be improved.

In the eyeglass system 1 of the fourth embodiment, the prediction unit 33 acquires information etc. stored in the user-related information storage unit 34f of the storage unit 34 in response to an inquiry from the user (or periodic processing by the server device 30). Thereby, the prediction unit 33 generates input data to the learning model and outputs power setting prediction information from the learning model to the operation device 20. The power setting prediction information may be information displayed on the operation device 20 to prompt the user to input, or may be information directly reflected in the power model information of the variable focus eyeglasses 10. Alternatively, the information may be information indicating whether or not to change the currently set power setting.

Although the data acquisition unit 31 in the fourth embodiment does not acquire gaze related information and control content information, which are information acquired by the variable focus eyeglasses 10, the data acquisition unit 31 is not necessarily limited to this aspect. That is, prediction accuracy may be improved by acquiring gaze related information and control content information and including these in data for constructing learning data.

### Fifth Embodiment

### <1. Overview>

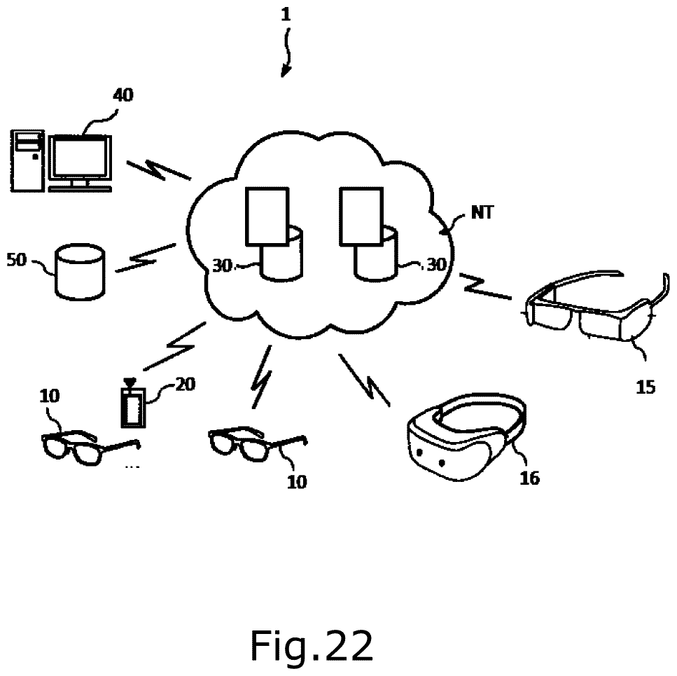

Next, an eyeglass system 1 according to a fifth embodiment of the present invention will be described. FIG. 22 is a diagram showing a schematic configuration of the eyeglass system 1 in the fifth embodiment. The eyeglass system 1 of the fifth embodiment includes smart glasses (AR glasses) 15 in addition to variable focus eyeglasses 10. These smart glasses are see-through glasses-type information devices that have the function of superimposing and displaying images or information on the real world that can be seen through it. Furthermore, the eyeglass system 1 of the fifth embodiment is configured to include a head-mounted display (VR glasses) 16 that is an eyeglass-type display device that serves as an image display device that covers the wearer's eyes.

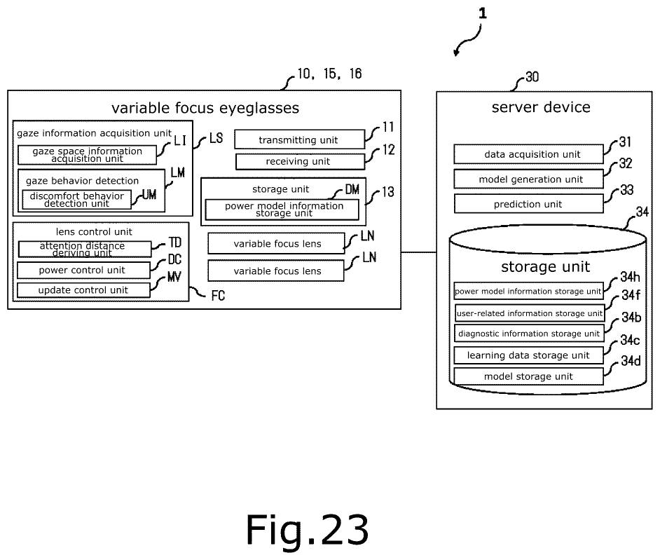

FIG. 23 is a diagram for explaining the functional configuration of the eyeglass system 1 of the fifth embodiment. The smart glasses 15 and the head-mounted display 16 also have substantially the same configuration as the variable focus eyeglasses 10, and are configured to include variable focus lenses LN and the like. Further, the variable focus eyeglasses 10 and the like have a power model information storage section DM, and the power control section DC in the lens control section FC controls the refractive index distribution of the variable focus lens LN with reference to the power model information.

It is desirable that the variable focus eyeglasses 10 or the like have an IPD (inter-pupillary distance) that can be adjusted manually or automatically.

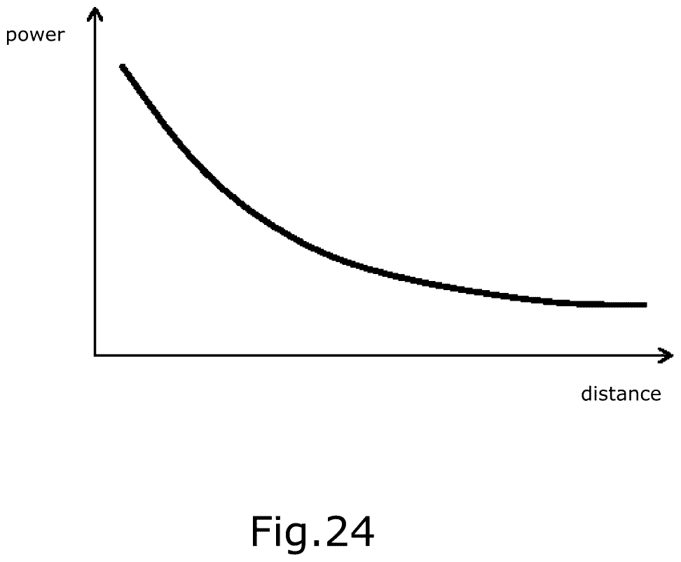

FIG. 24 is a diagram for explaining an example of power model information. The power model information is information for changing the refractive index distribution of the variable focus lens LN according to the situation and behavior of the wearer. That is, in FIG. 24, the power model information is information indicating the relationship between the distance to the object that the wearer of the variable focus eyeglasses 10 or the like is gazing at and the “spherical power (SPH)” of the variable focus lens LN that should be set by the wearer. Although one power model information is shown in FIG. 24, it is preferable that six power model information be held in the variable focus eyeglasses 10. These sixth power model information are information based on the left and right eyes, as well as “spherical power (SPH).” “astigmatism axis (AXIS),” and “astigmatism power (CLY).”

Currently; glasses-type information devices are widespread, and they are assumed to be used in a variety of situations. There may be cases in which individuals use their own glasses-type information devices, or there may be situations in which they borrow glasses-type information devices or the like from other people or other organizations. Here, the setting of the variable focus lens LN in a device such as a glasses-type information device is required to be different for each person, but it is inconvenient to be forced to input for customization for each occasion of use. For the above reasons, in the eyeglass system 1 of the fifth embodiment, power model information is held in the storage unit 34 (power model information storage unit 34h) of the server device 30. That is, the fifth embodiment is configured so that power model information can be shared between a plurality of eyeglasses via the server device 30, thereby improving convenience.

In addition, the power model information is not only configured to be able to be transmitted and received between glasses of the same type, but also to be shared between glasses that are different types of devices such as variable focus eyeglasses 10, smart glasses 15, and head mounts 16. By doing so, convenience is further improved. Note, it is preferable to that the variable focus eyeglasses 10 etc. of the eyeglass system 1 are caused to be able to receive and hold power model information transmitted from other variable focus glasses 10 etc. without necessarily going through the server device 30. When temporarily using a glasses-type information device or the like located in another location, convenience is improved by allowing the user to use the settings of the variable focus eyeglasses 10, etc. that he or she owns.

Further, the power model information in the fifth embodiment is updated as appropriate when the user uses the variable focus eyeglasses 10, etc., and this updated power model information is stored and held in the storage unit 34 of the server device 30, and is also shared within the glasses system 1.

The details of the eyeglass system 1 of the fifth embodiment will be described below, but the points that are different from the first embodiment will be mainly explained, and the points that are substantially the same will be omitted as appropriate.

### <2. About Variable Focus Glasses>

As shown in FIG. 23, the variable focus eyeglasses 10 and the like in the fifth embodiment configured to include a gaze information acquisition unit LS, a lens control unit FC, a transmission unit 11, a reception unit 12, a storage unit 13, and two variable focus lenses LN.

The gaze information acquisition unit LS is configured to include a gaze spatial information acquisition unit LI and a gaze behavior detection unit LM, and the gaze behavior detection unit LM is configured to include an unpleasant behavior detection unit UM. Further, the lens control unit FC is configured to include an attention distance deriving unit TD, a power control unit DC, and an update control unit MV. The lens control unit FC in the variable focus glasses 10 and the like controls the power such as the spherical power (SPH) in the variable focus lens LN based on the information acquired by the gaze information acquisition unit LS.

Further, the variable focus eyeglasses 10 and the like have means for accepting a user's instruction to request power model information (not shown in FIG. 23). When a user borrows variable focus eyeglasses 10 or the like from another person and uses the same, the user inputs into the variable focus eyeglasses 10 an instruction to request the server device 30 for the user's power model information. It goes without saying that the user's own power model information in this case is power model information that is synchronized to be the same as the power held by the user's own variable focus eyeglasses 10. The transmitting unit 11 of the variable focus eyeglasses 10 transmits a request instruction for power model information together with user identification information to the server device 30. The server device 30 acquires power model information corresponding to the user's identification information from the power model information storage unit 34h, and transmits it to the variable focus eyeglasses 10 that received the request instruction. The variable focus glasses 10 have acquired the power model information from the server device 30 by transmitting the request instruction retain the acquired power model information in the storage unit 13.

### (2-1) About Power Control

In the variable focus eyeglasses 10 of the fifth embodiment, a LiDAR sensor is disposed at one location near the base of the temple 103 on the rim 101 as the gaze spatial information acquisition unit LI. Further, in the outer frame portion of the variable focus lens LN of the rim 101, a light source and an image sensor are arranged as a gaze behavior detection unit LM. This light source is a light source that irradiates near-infrared light to a plurality of locations in both the left and right eyes, and the image sensor is an element that acquires reflected light from the cornea of the irradiated near-infrared light. Therefore, in the variable focus eyeglasses 10 of the fifth embodiment, gaze behavior is detected by eye tracking using the corneal reflection method. The gaze space information acquisition unit LI acquires three-dimensional point cloud data of a space covered by the wearer's gaze, and the gaze behavior detection unit LM detects the gaze direction of at least one eye of the wearer. The attention distance derivation unit TD in the lens control unit FC derives the distance to the object of interest to the wearer by deriving the point of interest in the three-dimensional point group data from the detected gaze direction. The power control unit DC controls the refractive index distribution within the variable focus lens LN so that the power corresponds to the distance to the object of interest calculated by the attention distance derivation unit TD. Note that this control operation by the power control section DC is of course performed based on the power model information held in the power model information storage section DM.

Since the variable focus eyeglasses 10 have the above-described configuration, when the direction of the gaze changes and the distance to the object of interest changes, power control of the variable focus lens is performed in accordance with power model information appropriate for the wearer.

Note that the mode of power control is not necessarily limited to the above. Therefore, for example, without using the three-dimensional information acquired by the gaze space information acquisition unit LI, the gaze direction of both eyes is detected from the eyeball image acquired by the gaze behavior detection unit LM, and the convergence angle is determined to determine the target of interest. The distance to the object may also be derived (in this case, information regarding the interpupillary distance of the wearer is input and held in the variable focus eyeglasses 10).

In addition, near miosis is known as near reflex, and the size of the pupils of both eyes (or one eye) is detected from the eyeball image, and the distance to the object of interest may be derived by calculating the pupil diameter. It is known that when accommodative power decreases due to presbyopia, miosis in near vision, which expands the depth of focus, increases. Therefore, for example, when the wearer is elderly; the distance to the object of interest may be derived based on the size of the pupil and the power of the variable focus eyeglasses 10 may be controlled.

### (2-2) Regarding Power Change Due to Detection of Specific Behavior

It is assumed that power control based on power model information and gaze behavior as described above may not always provide comfortable power control for the wearer due to deterioration of the wearer's visual acuity over time, physical condition, etc. For this reason, in the variable focus eyeglasses 10 of the fifth embodiment, control is performed to change the power determined by the power model information when a specific behavior by the wearer is detected. An example of this specific behavior is unpleasant behavior felt by the wearer due to mismatched power, and in this embodiment, it is detected by the eyeball image acquired by the gaze behavior detection unit LM.

Specifically, if only the gaze direction and the size of the pupil are to be detected, the eyeball image may be acquired at a first sampling rate of about several tens of Hz (10 Hz or more and 100 Hz or less). However, by increasing the sampling rate to a second sampling rate of several hundred Hz or more (200 Hz or more, or 600 Hz or more), it becomes easier to capture involuntary behavior. This involuntary behavior includes, for example, human emotions expressed in eye movements and changes in the subconscious mind. Image acquisition at high sampling rates consumes a lot of power. For this reason, it is preferable that the unpleasant behavior detection unit UM in the gaze behavior detection unit LM be able to detect unpleasant behavior by increasing the sampling rate in a relatively short predetermined period. That is, when performing power control of the variable focus eyeglasses 10 (or after reaching a target value for power control based on power model information), the sampling rate may be increased for a relatively short predetermined period. The plurality of sampling rates described above are switched based on, for example, information regarding the gaze obtained from the eyeball image (changes in pupil size, gaze direction, convergence angle, etc.). This may improve the efficiency of detecting unpleasant behavior.

When unpleasant behavior is detected, the power control unit DC controls the refractive index distribution to be different from the target value of power control based on the power model information. The power control unit DC searches for a state in which the unpleasant behavior is alleviated, for example, by changing the power stepwise or comprehensively within a predetermined power range. The power control unit DC changes the power to a level in which this unpleasant behavior has subsided. Alternatively, the power may be increased or decreased by determining the direction of the power change in which the unpleasant behavior decreases (each time the power is increased or decreased, it is determined whether or not the unpleasant behavior is attenuated). In this way, the power may be reduced to provide comfort for the wearer. (The power may be changed in stages so that the unpleasant behavior subsides after changing the power several times.)

It should be noted that when the cognitive load of discomfort is applied, the sympathetic nervous system increases and the pupil size tends to increase. Therefore, the unpleasant behavior detection unit UM may detect unpleasant behavior based on information regarding the pupil (pupil size, occurrence of vibration, etc.) obtained from the eyeball image. In this case, the distance to the wearer's object of interest is derived based on three-dimensional point cloud data and the gaze direction (or derived based on the convergence angle), and it is preferable to detect unpleasant behavior based on the size of the pupils. More specifically, information indicating the correspondence between the user's pupil diameter and convergence angle is periodically acquired and stored in the storage unit 13 in advance. On the other hand, the unpleasant behavior detection unit UM compares the pupil size corresponding to the convergence angle of both eyes detected by the gaze behavior detection unit LM with the pupil size obtained from the eyeball image. Then, based on this comparison operation, it may be determined whether or not unpleasant behavior is occurring.

### (2-3) About Updating Power Model Information

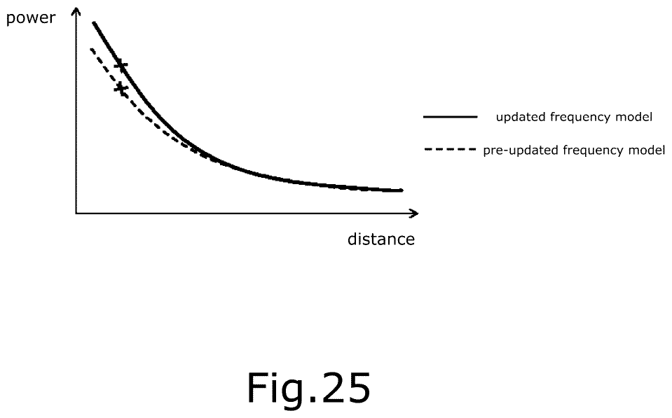

Further, the lens control unit FC of the variable focus eyeglasses 10 includes an update control unit MV, and the update control unit MV updates the power model information when the above-mentioned unpleasant behavior is detected. FIG. 25 is a diagram for explaining how power model information is updated. As shown by the cross mark in the figure, the power model information is newly updated while the power corresponding to the distance to the object that the wearer is paying attention to and the surrounding area are changed. More specifically; the above-mentioned new update of the power model information is performed by fitting so as to maintain the global trend of the distance to the object and the power. By updating the power model information, it is possible to provide the wearer of the variable focus eyeglasses 10 with a comfortable feeling of use.

The update control unit MV stores the updated power model information in the storage unit 13, and the power model information is transmitted from the transmission unit 11 to the server device 30. This makes it possible to share it with other variable focus eyeglasses 10, etc. in the eyeglass system 1, so that even when using variable focus eyeglasses 10 or glasses-type information devices that you do not own, you can use them smoothly

### (2-4) About the Flow of Automatic Focus Control

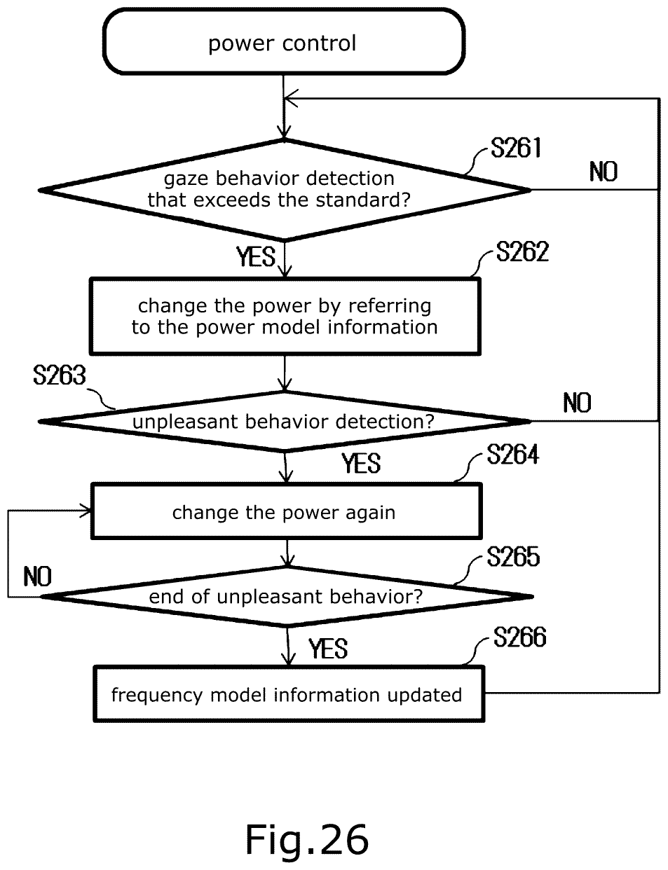

FIG. 26 is a diagram illustrating the flow of power control in the variable focus eyeglasses 10 of the fifth embodiment, and the flow of automatic focus control (autofocus control) by the lens control unit FC including updating of power model information.

First, when the power control unit DC in the variable focus eyeglasses 10 detects a gaze behavior that exceeds a certain standard accompanied by a change in the gaze target of the wearer (YES in S261), the process proceeds to step S262. In step 262, control is performed to change the refractive index distribution set in the variable focus lens LN with reference to the power model information. Specifically; in S262, the attention distance derivation unit TD derives the distance to the object that the wearer is paying attention to, and the power control unit DC outputs the power corresponding to the derived distance, based on the power model information.

After changing the power in S262, if unpleasant behavior of the wearer is detected (YES in S263), the power is changed again so that the power is in a different state from the power output according to the power model information. (S264). On the other hand, if no unpleasant behavior is detected (NO in S263), the process returns to S261 again and waits until gaze behavior that is higher than the standard is detected.

If the unpleasant behavior ends by changing the power again (YES in S265), the process updates the power model information. This process is executed based on information about the distance to the object of interest at the time the unpleasant behavior ended and the changed power. Furthermore, the updated power model information is stored in the power model information storage section DM (S266). The updated power model information is also transmitted to the server device 30. On the other hand, if the unpleasant behavior does not end (NO in S265), the process returns to S264 and the power is changed again. After S266, the process returns to S261 again to enter a standby state for gaze behavior detection, and a series of automatic focus control flows are repeated.

### (2-5) Others

Note that as the unpleasant behavior, a state in which the muscles around the eyes are tense may be detected using an electrooculography sensor for detecting the state of the muscles around the eyes.

Note that the specific behavior that can be a trigger for updating the power model information by changing to a refractive index distribution that does not follow the power model information does not necessarily have to be an involuntary behavior that is unpleasant for the wearer. Therefore, for example, the voluntary behavior of the eyes, such as repeating blinking multiple times, is predetermined as a specific behavior, and the gaze behavior detection unit LM may be performed control to change the power by detecting such specific behavior. In this case, the wearer continues the specific behavior such as blinking until the power change control is satisfactory. Further, the specific behavior does not necessarily have to be a gaze behavior, but may be a behavior that involves any movement, such as detected by a 6-axis sensor built into the rim 101 of the variable focus eyeglasses 10, for example. Furthermore, the rim 101 may include a sensor that detects a behavior such as a gesture by the wearer as a specific behavior.

Note that the power model information is not necessarily limited to that shown in FIG. 24. For example, the relationship between the convergence angle of the wearer and the power that the wearer should set may be shown, or the relationship between the pupil size of the wearer and the power that the wearer should set may be shown. Alternatively, the rim 101 may have a built-in sensor that acquires environmental light, and the information may be information indicating the relationship between the acquired environmental light, the pupil size, and the diopter that should be set by the wearer.

Note that, as shown in FIG. 22, the variable focus eyeglasses 10 may be connected to the server device 30 via the operation device 20, and similarly, the smart glasses 15 and the head-mounted display 16 may also be connected to the server device 30 via the operation device 20. The variable focus eyeglasses 10 and the like may be configured such that initial input and update input of the power model information can be performed by inputting information from the user via the operation device 20 (or directly). When the power model information is updated by the operation device 20, the display screen is similar to the power setting support mode of the second embodiment (FIGS. 15A and 15B), and the distance to the object of interest (or convergence angle or pupil size etc.) may be displayed so that input from the user of power setting information corresponding to the distance may be accepted. Similarly, the image display device included in the head-mounted display 16 may output a display such as the power setting support mode (FIGS. 15A and 15B) of the second embodiment. Further, the smart glasses 15 may output a display such as the power setting support mode so as to be superimposed on the real world, so that the user's input may be accepted. As shown in FIG. 15B, the mode that accepts input regarding the direction of the astigmatic axis (AXIS) is executed on the head-mounted display 16 or smart glasses 15, and an image including a figure indicating the direction of the astigmatic axis is displayed to the user. This simplifies the work of setting the astigmatism axis. Note that the head-mounted display 16 has a built-in optometry machine that acquires data such as wavefront aberration of the wearer's eyes as optometry information, updates power model information based on this optometry information, and the power model information updated may also be shared in the server device 30. Note that, as in the fourth embodiment, the server device 30 may include an optometry information storage section 34g.

### <3. About Learning Data and Learning Models>

Below, machine learning executed by the eyeglass system 1 according to the fifth embodiment and a learning model generated by the machine learning will be explained.

The server device 30 includes a data acquisition unit 31, a model generation unit 32, a prediction unit 33, and a storage unit 34. Further, the data acquisition unit 31 acquires diagnostic information regarding the diagnosis result of a specific disease for the user by transmission from the medical worker client device 40 or the medical related database 50, and accumulated the diagnostic information in the diagnostic information storage unit 34b. Furthermore, lifestyle information, biological information, and attribute information are accumulated in the user-related information storage unit 34f.

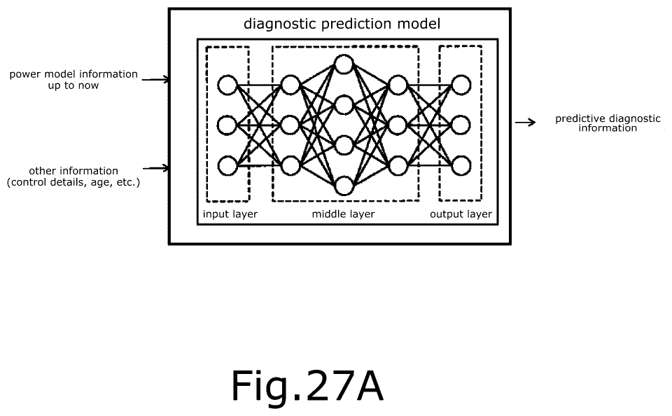

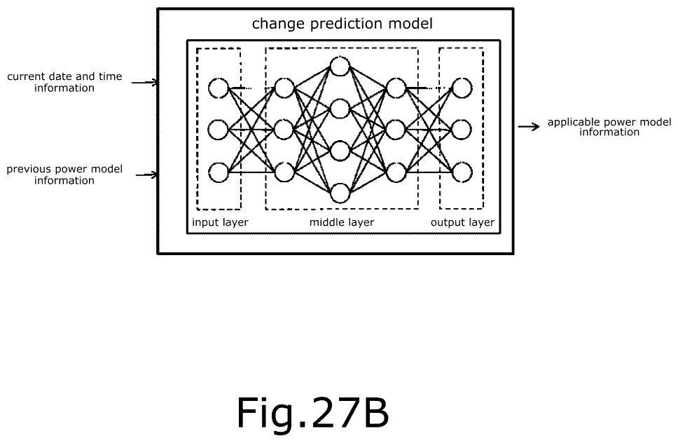

FIGS. 27A and 27B are diagrams for explaining two types of learning models generated by the eyeglass system 1 of the fifth embodiment, and these will be explained below.

(3-1) Learning Model that Predicts the Occurrence of Specific Diseases and Symptoms

The storage unit 34 of the server device 30 stores power model information and diagnostic information regarding specific diseases (eye disease, dementia, etc.) in association with user IDs. Furthermore, the learning data storage unit 34c holds a learning data-set constructed to generate a model (diagnosis prediction model) for predicting the occurrence of a specific disease. “Teacher data” in the learning data-set is diagnostic information, and “input data” includes power model information and the like.

In the power model information storage unit 34h, the power model information is stored in association with the user ID and information indicating the updated timing. Further, the diagnostic information, which is teacher data, is also stored in the diagnostic information storage unit 34b in association with diagnostic timing information indicating when the user received the diagnosis. In machine learning using a learning data-set should use features. This feature amount is generated, for example, based on power model information corresponding to the time indicated by the diagnosis time information and one or more power model information accumulated before the time indicated by the diagnosis time information. It is thought that prediction accuracy can be improved by utilizing such feature quantities that include the user's power variations up to the time of receiving a diagnosis in machine learning that generates a learning model. Note that other data used as input data for the learning data-set includes attribute information such as gender and age, biological information such as height and weight, and lifestyle information such as control content information of the variable focus eyeglasses 10 and gaze related information. (Sec FIG. 16 and its explanation). Further, prediction accuracy can also be expected to be improved by utilizing feature quantities that include control details of the variable focus eyeglasses 10, tendencies of the user's gaze behavior, etc. in machine learning for generating a learning model.

The model generation unit 32 generates a diagnostic prediction model by performing machine learning using the learning data held in the learning data storage unit 34c, and stores it in the model storage unit 34d.

FIG. 27A is a diagram for explaining an example of a diagnostic prediction model that outputs diagnostic prediction information. As with the input data to the learning data-set, the input data at the time of prediction by the diagnostic prediction model includes the power model information up to the present time and the other information described above. Note that the power model information up to the present time is current power model information and one or more power model information accumulated in the past before the current time.

The prediction unit 33 generates input data to a diagnostic prediction model in response to a request from a user of the variable focus eyeglasses 10, etc., and further performs a process of converting it into information including multidimensional feature amounts corresponding to the predictive diagnostic model. It also performs arithmetic processing using a predictive diagnostic model and outputs diagnostic predictive information to be sent to a terminal managed by the user. The eyeglass system 1 in the fifth embodiment is a predictive diagnostic system that outputs diagnostic predictive information in response to a request from a user.

(3-2) Learning Model that Predicts Changes in Power Model Information

The storage unit 34 of the server device 30 holds a learning data-set different from the learning data-set for generating a diagnostic prediction model. This other learning data-set is a learning data-set for generating a learning model (change prediction model) for predicting changes in frequency model information in the learning data storage unit 34c. In the power model information storage unit 34h, the power model information is stored in association with the user ID and information indicating the updated timing. The change prediction model is a learning model for outputting information regarding power model information that is currently suitable for the user. In addition, the “teacher data” of the learning data-set for generating the change prediction model is power model information, and the “input data” includes multiple power models accumulated in the past than the power model information used as the training data.

In addition, the “input data” in the learning data-set may be included information indicating the update date and time of the power model information used as the teacher data, and at least one power model information among the plurality of power model information. This at least one piece of power model information is information that is included in the previous input data and indicates the update date and time of the power model information that is closest to the update date and time of the power model information used as teacher data. In machine learning for generating a change prediction model, the accuracy of the change prediction model can be improved by using the following feature quantities. The feature quantity is information indicating the length of a period determined based on the update date and time of the power model information used as the teacher data and the update date and time closest to the teacher data among the power model information included in the input data. Furthermore, information including temporal relationships between power model information can also be used. Note that, as shown in FIG. 27A, the “input data” in the learning data-set may include attribute information, biological information, and lifestyle information such as control content information and gaze related information of the variable focus eyeglasses 10 etc. Note that the accuracy of the change prediction model may be improved by including at least one power model information, other lifestyle information, etc. as “input data” of the learning data-sct. Note that while the input data in the learning data-set includes some (or a plurality of) pieces of power model information, the teacher data includes power model information that has been updated after the plurality of power model information, and the power model information updated at the latest timing among the plurality of power model information may be included. The teacher data may be information regarding changes such as difference values between power model information updated after the plurality of the power model information and any power model information among the plurality of the power model information. Note that the above any power model information is specifically power model information updated at the latest timing.

The model generation unit 32 generates a change prediction model by machine learning using the learning data-set held in the learning data storage unit 34c as described above, and stores it in the model storage unit 34d.

FIG. 27B is a diagram for explaining an example of a change prediction model that outputs power model information that can be adapted to the current time. The input data for the change prediction model in FIG. 27B include current date and time information indicating the current point in time, and previous power model information (specifically: multiple power model information updated in the past than the current point in time and their information indicating the update timing) is included.

For example, the prediction unit 33 generates input data to the change prediction model in response to a request from a user of the variable focus eyeglasses 10 or the like (or periodically), and performs the following process. That is, this process is a process of converting into information including multidimensional features corresponding to the change prediction model and an arithmetic process using the change prediction model, and the prediction unit 33 outputs the prediction result to the variable focus eyeglasses 10. The eyeglass system 1 of the fifth embodiment is a power model information prediction system that outputs information regarding power model information suitable for the user in response to a request from the user. Note that the information output from the change prediction model may be power model information that is compatible with the user at the time of the request, or, for example, information indicating whether or not the power model information of the user at the time of the request is compatible. Note that the power model information that is sent to the variable focus eyeglasses 10 and that is applicable at the present time may be separately held in the storage unit 13 of the variable focus eyeglasses 10 as power model candidate information. Further, for example, the power model information may be updated efficiently by referring to the power model candidate information when a specific behavior is detected. There are multiple types of power model information held by the variable focus eyeglasses 10, such as “SPH” and “CLY” for both the left and right eyes, and by providing power model candidate information, user convenience can be improved.

Note that in the server device 30 of the fifth embodiment, optometry information can be accumulated in the optometry information storage section 34g. That is, optometry information may be included in the input data in the learning data-sets of the “diagnosis prediction model (FIG. 27A)” and “change prediction model (FIG. 27B)” described above. It is thought that prediction accuracy will improve by using feature quantities that reflect optometry information in machine learning.

### [Regarding Modification 2 of Variable Focus Eyeglasses 10]

The first to fifth embodiments of the eyeglass system 1 have been described above. Below, a modification 2 of the variable focus eyeglasses 10 that can be applied to the eyeglass system 1 such as the first embodiment will be further described.

The variable focus eyeglasses 10 have two variable focus lenses LN on the left and right, and the variable focus lenses LN are configured to include two liquid crystal elements LU1 and LU2, as shown in FIG. 3A. In the first embodiment and the like, the same control signal is input to the liquid crystal elements LU1 and LU2, and the focal lengths are controlled to be the same, but in the modification 2, different control signals are input to the liquid crystal elements LU1 and LU2. That is, the liquid crystal elements LU1 and LU2 are independently controlled by the lens control unit FC. The variable focus eyeglasses 10 of the modification 2 differ from the variable focus eyeglasses 10 of the first embodiment and the like in these points, and these differences will be mainly described below. Further, descriptions of points that are similar to the variable focus eyeglasses 10 of the first embodiment and the like will be omitted as appropriate.

In the variable focus lens LN of modification 2, it is possible to generate different refractive index distributions between the liquid crystal element LU1 and the liquid crystal element LU2 so that they can be controlled to have different focal lengths. In other words, the liquid crystal elements LU1 and LU2 are configured to be able to set focal lengths corresponding to two objects placed at different distances from the variable focus eyeglasses 10. Furthermore, the alignment directions of the liquid crystal element LU1 and the liquid crystal element LU2 are configured to be substantially perpendicular to each other. Therefore, the light from the object that can be clearly seen by the liquid crystal element LU1 and the light from the object that can be clearly seen by the liquid crystal element LU2 are captured by the user's retina in an approximately half-to-half ratio. As a result, multifocal glasses having a focal length corresponding to s-polarized light and a focal length corresponding to p-polarized light are realized.

The variable focus eyeglasses 10 have the first to third modes as in the first embodiment, and also may have a mode in which different refractive index distributions and focal lengths are generated in two liquid crystal elements whose alignment directions are perpendicular to each other. (Hereinafter, in this specification, this mode is also referred to as sp multifocal mode). Furthermore, depending on the distance measured by the laser distance measuring module, for example, a transition may be made to the sp multifocal mode between the first mode and the second mode. In this case, in the sp multifocal mode that transitions between the first mode and the second mode, may be set to the liquid crystal element LU1 has the same power setting as the first mode, and may be set to the liquid crystal element LU2 has the same power setting as the second mode. In addition, this sp multifocal mode may be transition depending on an input operation from the operation device 20 by the user or a separately determined condition, separately from the first to third modes that are switched based on gaze related information. Note that when the variable focus glasses 10 are controlled by power model information as shown in FIG. 24, a state may exist in which different refractive index distributions occur in two liquid crystal elements whose alignment directions are perpendicular to each other depending on the distance to the gaze object.

Further, as the variable focus eyeglasses 10, when controlling the power of the variable focus lens LN based on power model information may be controlled as follows. That is, one liquid crystal element may be controlled so that the other liquid crystal element reaches the target power value with a delay. Specifically: when a transition occurs between the first mode and the second mode (transition between the normal mode and not the multifocal mode), it is also possible to cause one liquid crystal element to change with a delay (following) the other liquid crystal element. By changing with a delay in this way, it becomes easier to understand that the power setting of the variable focus eyeglasses 10 has changed, and the user's usability is improved. Further, even in a case where a transition to the sp multifocal mode occurs, the liquid crystal element LU1 may be configured to change with a delay with respect to the liquid crystal element LU2. The delay between one liquid crystal element and the other liquid crystal element may be a configuration that delays the start timing of changes in the refractive index distribution or focal length, or lengthens the period in which these changes occur.

Further, the variable focus eyeglasses 10 may be configured such that, for example, the refractive index distributions of two liquid crystal elements whose alignment directions are perpendicular to each other are controlled by different algorithms based on information regarding the user's gaze acquired by the gaze information acquisition unit LS. Specifically, a configuration can be considered in which the power settings of the two liquid crystal elements are transitioned by different the conditions depending on the distance measured by the laser distance measuring module. Further, for example, the refractive index of the liquid crystal element LU1 is changed immediately (for example, after 0.3 seconds) according to the switching conditions of the first to third modes, on the other hand, the refractive index of the liquid crystal element LU2 may be changed when the switching condition is maintained for a predetermined period (for example, 1.2 seconds or more). In addition, while controlling the power setting of one liquid crystal element LU1 using information acquired by the laser ranging module (gaze spatial information acquisition unit), the power setting of the other liquid crystal element LU2 may be controlled by using information acquired by a potential sensor (gaze behavior detection unit) on the nose pad D1. In this way: the two liquid crystal elements LU1 and LU2 can flexibly respond to the movement of the user's gaze by executing separate controls, thereby reducing the sense of discomfort when the power changes.

### [About Modification 3 of Variable Focus Eyeglasses 10]

Further, below, a modification 3 of the variable focus eyeglasses 10 that can be applied to the eyeglasses system 1 such as the first embodiment will be further described.

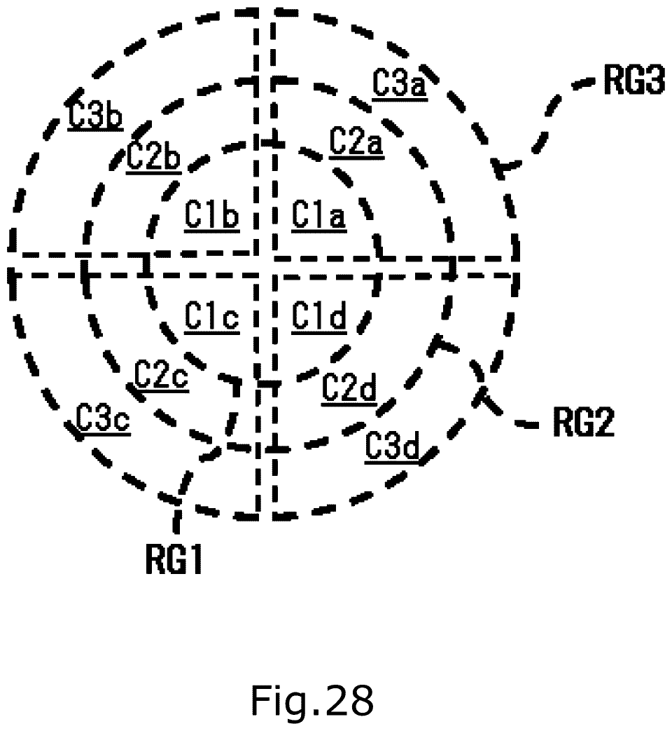

FIG. 28 is a diagram showing the arrangement of the common input section and the annular region in the liquid crystal elements LU1 and LU2 in the modification 3. As shown in the figure, the liquid crystal element LU1 of the modification 3 has 12 common input sections C1a to C3a, C1b to C3b, C1c to C3c, and C1d to C3d, (see FIG. 8A), as in the first embodiment, and further has three annular regions RG1, RG2, and RG3. Specifically, the first annular region RG1 has four common input parts C1a to C1d continuous in the circumferential direction and shaped like a ring, the second annular region RG2 has four common input parts C1a to C1d, and the third annular region RG3 has four common input portions C3a to C3d.

The variable focus eyeglasses 10 of modification 3 has a mode (hereinafter also referred to as region division multifocal mode) in which at least two of the three annular regions are controlled to have different focal lengths. This is different from the variable focus eyeglasses 10 of the first embodiment. Hereinafter, such differences will be mainly described, and descriptions of points that are similar to the variable focus eyeglasses 10 of the first embodiment and the like will be omitted as appropriate.

In this region-division multifocal mode, for example, a transition may also occur between the first mode and the second mode, or between the second mode and the third mode, depending on the distance measured by the laser ranging module. For example, in a region-divided multifocal mode provided between the first mode and the second mode, a control signal may be input in the first annular region RG1 and the third annular region RG3 so as to correspond to the power setting of the first mode, and at the same time, a control signal may be input in the second annular region RG2 to correspond to the power setting of the second mode. In addition, the region division multifocal mode may be transition in response to an input operation from the operation device 20 by the user or a separately determined condition, separately from the first to third modes. Note that when the variable focus eyeglasses 10 are controlled by power model information as shown in FIG. 24, there may be a state in which at least two of the plurality of annular regions are controlled to have different focal lengths. Note that such control is performed according to the distance to the gaze target.

Further, when controlling the power based on the power model information, the lens control unit FC may perform the control as follows. That is, one of the annular regions may be controlled so that the focal length reaches the target value with a delay with respect to the other annular region. Control to delay each annular region in this way may be performed, for example, in the case changing the focal length of each annular region using the power settings of the first to third modes as target values. Further, it may be performed in the case changing power based on power model information. By changing with a delay in this manner, it becomes easier to understand that the power setting of the variable focus glasses 10 is changing, and thus the user's usability is improved. Further, the annular region in which the delay is caused may be generated in an annular region located farther from the optical axis LA than in an annular region located closer to the optical axis LA. Furthermore, the delay generated in the annular region may be one that delays the start timing of changing the focal length, or one that delays the change in focal length.

Further, for example, even if the target value of the focal length is different between the first annular region RG1, the second annular region RG2, and the third annular region RG3 (state of region division multifocal mode), the focal length of the third annular region RG3 may change later than that of the first annular region RG1.

In addition, when the lens control unit FC is controlled so that the focal length of the annular region (first annular region RG1) disposed close to the optical axis becomes the target value, the following control may be performed. That is, the control may be performed so that the focal length oscillates within a predetermined range including the target value for a predetermined period in an annular region (e.g., third annular region RG3) located far from the optical axis LA. By doing so, the usability for the user can also be improved.

Further, in an annular region far from the optical axis LA (for example, the third annular region RG3), the lens control unit FC may perform the following operation instead of causing a delay in focal length target value control or generating vibration. That is, control may be performed to intentionally lower the image quality of the lens and make it opaque for a certain period of time. By doing so, the usability for the user can also be improved. In addition, as a control for degrading image quality, for example, voltages whose phases are shifted to some extents are input to the first electrode E1 and the second electrode E2 of each unit-electrode U1 belonging to one or more common input section that constitute the annular region. Further, the frequency of the voltage input to the low potential side of the first electrode E1 and the second electrode E2 may be higher than the frequency of the voltage input to the high potential side. By doing so, the potential gradient formed in the space between the first electrode E1 and the second electrode E2 can be relatively easily disturbed, and the portion of the common input section that has a lens function is smaller than the portion that does not have a lens function, and as a result, visibility can be worsened.

Further, in the variable focus eyeglasses 10, the two annular regions may be controlled by different algorithms based on information regarding the user's gaze acquired by the gaze information acquisition unit LS, for example. Specifically, it is conceivable to change the focal length of the two annular regions by changing the conditions depending on the distance measured by the laser distance measuring module. Further, for example, the response speed of the power control based on gaze behavior may be made different between the annular regions, or the focal lengths of the first annular region RG1 and the second annular region RG2 may be changed. That is, the focal lengths of the previous regions RG1 and RG2 are changed immediately (for example, after 0.3 seconds) according to the switching conditions for the first to third modes, and furthermore, the focal length of the third annular region RG3 may be changed when the above condition is maintained for a predetermined period (for example, 1.2 seconds or more). Furthermore, while controlling the focal length of one annular region using information acquired by the gaze spatial information acquisition unit, the focal length of another annular region may be controlled using information acquired by the gaze behavior detection unit. By doing so, it is possible to reduce the sense of discomfort when the power changes.

Note that in the variable focus lens LN, the sp multifocal mode and the area division multifocal mode may be applied simultaneously. Note that in the case of having three annular regions as shown in FIG. 28, by simultaneously applying two multifocal modes, a multifocal lens compatible with a maximum of six types of focal points can be realized.

### [Regarding Modification 4 of Variable Focus Eyeglasses 10]

Further, in the following, a modification 4 of the variable focus eyeglasses 10 that can be applied to the eyeglasses system 1 such as the first embodiment will be further described.

In the variable focus eyeglasses 10 of the first embodiment, astigmatism is generated by controlling fan-shaped areas facing each other via the optical axis LA so that they have the same focal length, etc., to correct eye astigmatism. However, the modification 4 is configured to generate astigmatism in the variable focus lens LN by a liquid crystal element that functions as a Fresnel type cylindrical lens (linear Fresnel lens). Modification 4 is mainly different from the first embodiment in these points, and descriptions of points similar to the variable focus eyeglasses 10 of the first embodiment will be omitted as appropriate.

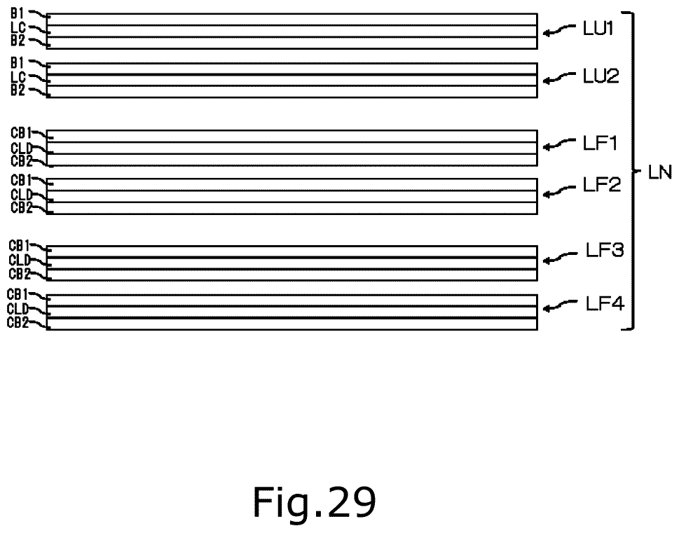

FIG. 29 is a diagram for explaining the configuration of the variable focus lens LN according to modification 4, in which, in addition to liquid crystal elements LU1 and LU2 similar to the first embodiment, a liquid crystal elements LF1 to LF4 that functions as a linear Fresnel lens is included. Further, the liquid crystal elements LF1 to LF4 include a transparent substrate CB1, a transparent substrate CB2, and a liquid crystal layer CLD, and have a structure similar to that shown in FIG. 5. That is, a plurality of unit-electrode U1 each having a rectangular planar shape are arranged in parallel and extending linearly.

Specifically: the liquid crystal elements LF1 to LF4 are configured such that the width of the unit-electrode U1 becomes narrower as the distance from the center increases (see FIG. 6). And in a cross section of the liquid crystal layer CLD in a direction perpendicular to the extending direction of each unit-electrode U1, a refractive index distribution having a convex and concave Fresnel lens shape can be formed.

Furthermore, in the liquid crystal elements LF1 and LF2, the extending direction of the unit-electrode U1 is the same direction (first direction) and the orientation direction of the liquid crystal layer CLD is perpendicular to each other, and the liquid crystal elements LF3 and LF4 are also similarly, the extending direction of the unit-electrode U1 is in the same direction (second direction), and the alignment directions of the liquid crystal layer CLD are perpendicular to each other. On the other hand, the liquid crystal elements LF1 and LF2 and the liquid crystal elements LF3 and LF4 are arranged in a relationship such that the extending direction of the unit-electrode U1 forms approximately 45 degrees (a relationship in which the first direction and the second direction form approximately 45 degrees). Thereby: it is possible to generate the astigmatism in the variable focus lens LN and to rotate the eye astigmatism axis.

Further, in the variable focus lens LN in modification 4, the orientation directions in the liquid crystal layer of the liquid crystal elements LU1, LF1, and LF3 are aligned in a predetermined direction, and the orientation directions in the liquid crystal layer of the liquid crystal elements LU2. LF2, and LF4 are aligned perpendicularly from the predetermined direction. Therefore, by using two liquid crystal elements LF1 and LF3 whose orientation directions are aligned and whose unit-electrode U1 extend approximately 45 degrees, it is possible to generate astigmatism or rotate the eye astigmatic axis in the polarized light component corresponding to the orientation direction.

Note that the liquid crystal elements LF1 and LF3 whose unit-electrodes U1 extend in different directions are, for example, arranged so as to avoid a relationship in which the unit-electrode U1 extend in substantially parallel or substantially perpendicular directions. By doing so, control such as rotating the eye astigmatic axis of the variable focus lens LN becomes possible. In this way: the two optical elements that function as linear Fresnel lenses and can control the refractive index distribution are configured so that their focal axes are arranged avoiding perpendicular or parallel relationships, and by controlling the power of the lens using components in two directions, it becomes possible to rotate axis of the astigmatism. Therefore, for example, the included angle between the first direction and the second direction may be greater than or equal to 20 degrees and less than or equal to 70 degrees, or may be greater than or equal to 30 degrees and less than or equal to 60 degrees.

Note that as a means for generating astigmatism in the variable focus lens LN, for example, may be combined a liquid crystal element LF1 etc. that functions as a linear Fresnel lens and a configuration in which focus control is performed for each pair of fan-shaped areas in the liquid crystal elements LU1 and LU2 (see embodiment 1).

Note that when the lens control unit FC in the variable focus lens LN receives an input for changing the astigmatic power or the astigmatic axis by the liquid crystal elements LF1 to LF4, in the liquid crystal elements LU1 and LU2, by inputting correction control according to this input, the spherical power (SPH) that is the original target value may be realized.

### [Regarding Modification 5 of Variable Focus Eyeglasses 10]

Further, below, a modification 5 of the variable focus eyeglasses 10 that can be applied to the eyeglasses system 1 such as the first embodiment will be further described.

The variable focus eyeglasses 10 of modification 5, like modification 4, include a plurality of liquid crystal elements capable of generating a linear Fresnel lens-like refractive index distribution, thereby making it possible to generate astigmatism and correct eye astigmatism. However, there are differences in the extending direction of the unit-electrode U1 of the liquid crystal element, etc. Hereinafter, points that are different from modification 4 will be mainly explained, and descriptions of points that are similar to modification 4 will be omitted as appropriate.

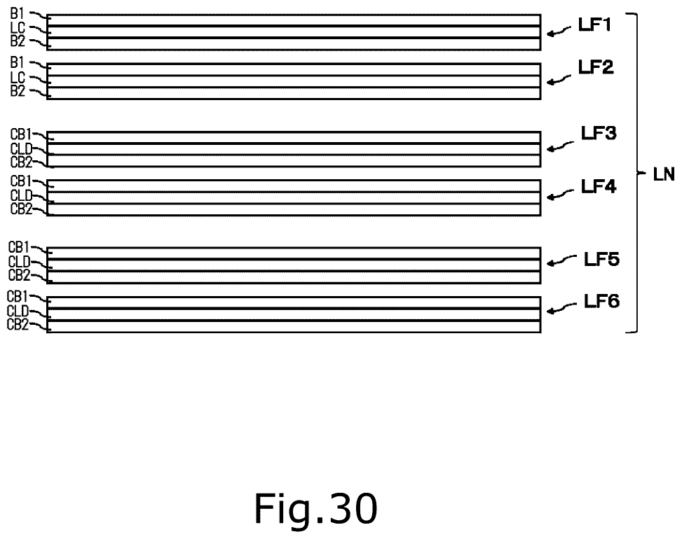

FIG. 30 is a diagram for explaining the configuration of a variable focus lens LN according to modification 5, and the variable focus lens LN is configured to include liquid crystal elements LF1 to LF6 that function as linear Fresnel lenses. In the liquid crystal elements LF1 and LF2, the extending direction of the unit-electrode U1 is the same (first direction), and the orientation directions are perpendicular to each other. Similarly, the extending directions of the unit-electrode U1 of the liquid crystal elements LF3 and LF4 is the second direction, and the orientation directions are perpendicular to each other, and the extending direction of the unit-electrode U1 of the liquid crystal elements LF5 and LF6 is the third direction, and the orientation directions are perpendicular to each other.

The liquid crystal elements LF1 to LF6 are arranged such that the narrow angle formed by each of the first direction, the second direction, and the third direction is an angle of 50 degrees or more and 70 degrees or less, and in modification 5, the angle is configured to be approximately 60 degrees. By arranging the liquid crystal elements LF1 to LF6 capable of generating a linear Fresnel lens-like refractive index distribution in this manner, it becomes easy to control the generation of refractive index distributions corresponding to various eye astigmatic powers and eye astigmatic axes.

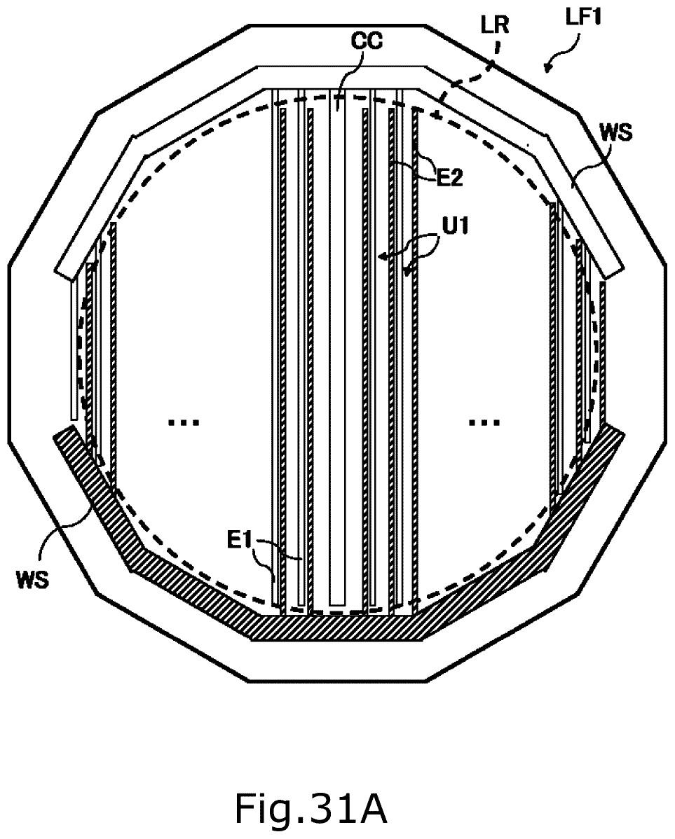

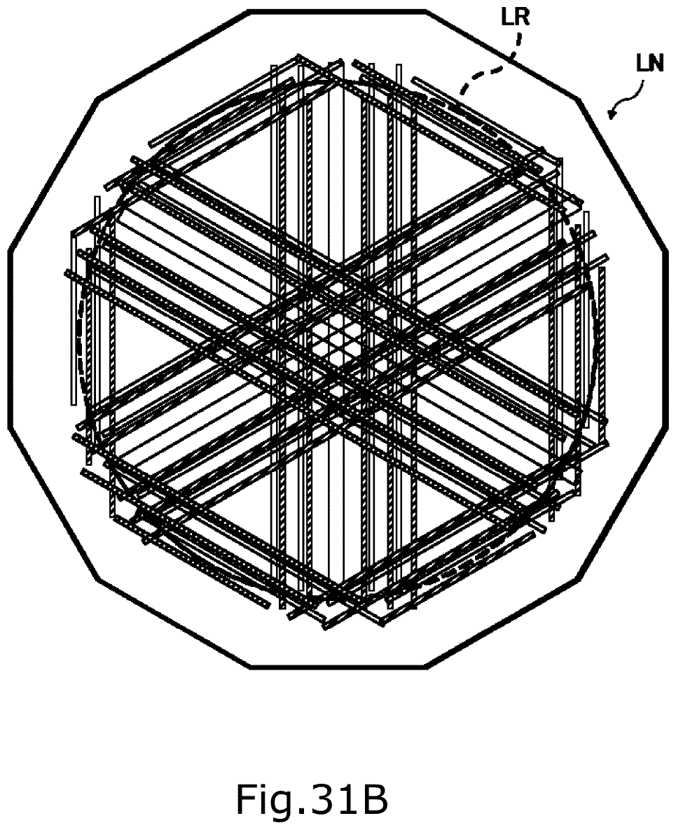

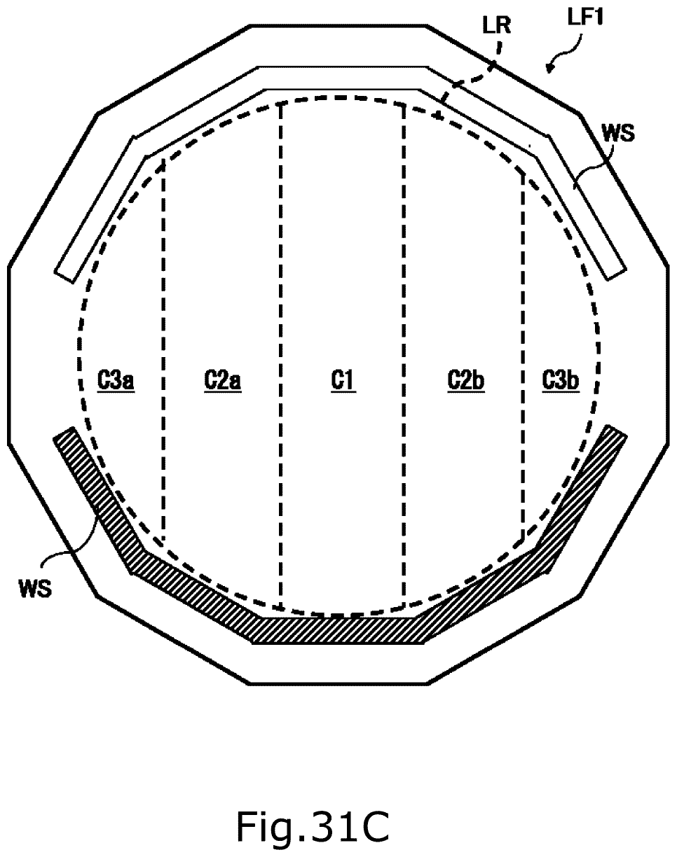

FIG. 31A is a diagram for explaining the planar layout of the unit-electrode U1 of the liquid crystal element LF1 in modification 5, and FIG. 31B is a schematic diagram showing how the unit-electrodes U1 (first electrode E1, second electrode E2) in the liquid crystal elements LF1 to LF6 of the variable focus lens LN extend in respective directions. Since FIG. 31B is a schematic diagram, some structures and symbols are omitted. Further, FIG. 31C is a diagram showing how five common input sections C1, C2a, C2b, C3a, and C3b of the liquid crystal element LF1 are arranged.

In the liquid crystal element LF1, as shown in FIG. 31A, the unit-electrode U1 extends in the vertical direction in the figure, and the width of the unit-electrode U1 becomes narrower from the center toward both ends. Further, a core electrode CC is arranged at the center of the lens region LR where a refractive index distribution occurs. The lens region LR is a generally circular region, and the lead wire region WS is arranged along a regular dodecagonal region circumscribing the region. There are two types of lead wire regions WS: one in which lead wires that supply voltage to the first electrode E1 group are arranged, and one in which lead wires that supply voltage to the second electrode E2 group are arranged. Each of the two lead wire regions WS has five wirings corresponding to the five common input sections, but the wiring notation is not shown in FIG. 31A.

Furthermore, the liquid crystal elements LF2 to LF6 have the same layout as the liquid crystal element LF1, and have a layout in which the external shapes and lens regions LR are approximately the same even when rotated by 60 degrees or 120 degrees. For this reason, although the liquid crystal elements LF1 to LF6 of modification 5 have an approximately regular dodecagonal shape in their external shape and the area in which the unit-electrode U1 is formed, the shape is not necessarily limited to such a shape. Therefore, the outer shape of the liquid crystal element LF1 and the formation area of the unit-electrode U1 may be, for example, circular or regular hexagonal.

Note that, as shown in FIG. 31C, the liquid crystal elements LF1 to LF6 that function as linear Fresnel lenses have five common input parts C1, C2a, C2b, C3a, and C3b, which are band-shaped areas. Similarly to the annular region of the modification 3, multifocal control by region division may be performed on the band-shaped areas corresponding to the three common input sections C1, C2a, and C3a having different distances from the optical axis.

Note that the variable focus lens LN of the modification 5 may also include liquid crystal elements LU1 and LU2 similarly to the variable focus lens LN of the modification 4. By combining liquid crystal elements LU1 and LU2 (see FIG. 3B) similar to those in the first embodiment and liquid crystal elements LF1 to LF6 that function as linear Fresnel lenses with different area division modes, various control of the refractive index distribution in the variable focus lens LN can be performed, and as a result, it is possible to respond to various eye conditions of the wearer.

### [About Modification 6 of Variable Focus Eyeglasses 10]

Further, below, a modification 6 of the variable focus eyeglasses 10 that can be applied to the eyeglasses system 1 such as the first embodiment will be further described.

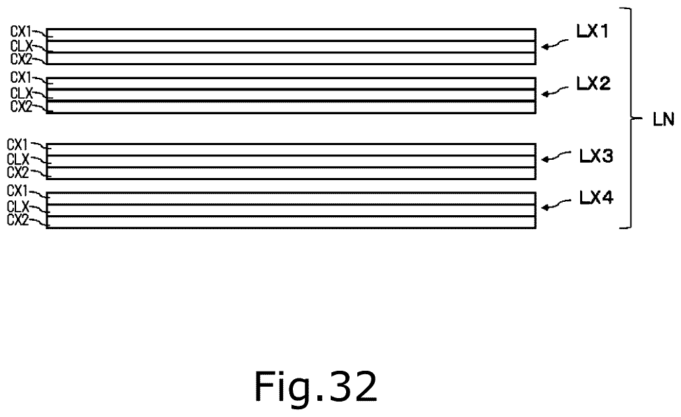

FIG. 32 is a diagram for explaining the schematic configuration of a variable focus lens LN of modification 6, and the variable focus lens LN is configured to include four liquid crystal elements LX1 to LX4. The liquid crystal elements LX1 to LX4 are configured to include two transparent substrates CX1 and CX2 that sandwich a liquid crystal layer CLX.

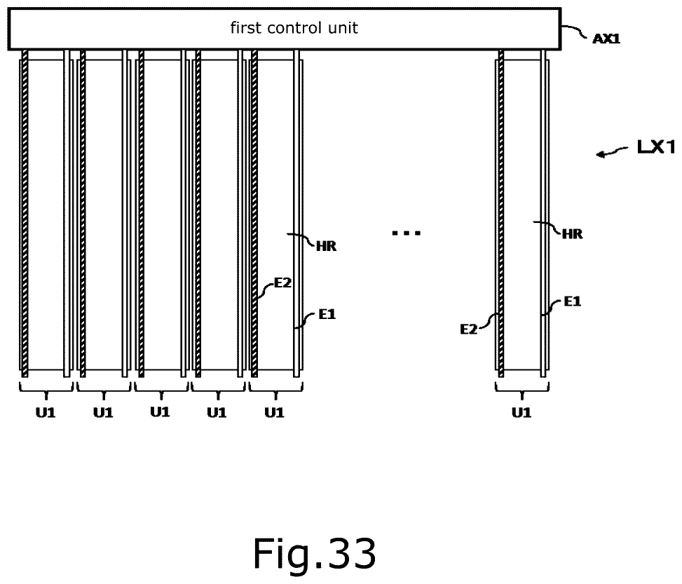

FIG. 33 is a schematic diagram of the planar configuration of the liquid crystal element LX1, and the liquid crystal elements LX2 to LX4 also have the same configuration (in the liquid crystal elements LX3 and LX4, a second control unit AX2 is arranged in place of the first control unit AX1). As shown in FIG. 33, in the liquid crystal element LX1, a plurality of unit-electrodes U1 having substantially the same width extend linearly and are arranged adjacent to each other. Each unit-electrode U1 has a first electrode E1 and a second electrode E2, and also includes a resistance layer HR disposed between them in a plan view.

More specifically, each unit-electrode U1 of modification 6 is arranged at a pitch of 150 μm, and the length of each unit-electrode U1 is about 30 mm. At the boundary between two adjacent unit-electrodes U1, the resistance layer HR is divided, and the space between the first electrode E1 and the second electrode E2 has a width of 5 μm. Further, the liquid crystal element LX1 has 200 unit-electrodes U1, and the variable focus lens LN generates a refractive index distribution within a 30 mm square rectangular area by the four liquid crystal elements LX1 to LX4.

In the liquid crystal elements LX1 and LX2, the extending direction of the unit-electrode U1 is the same (first direction), and the orientation directions in the liquid crystal layer CLX are orthogonal to each other. Further, in the liquid crystal elements LX3 and LX4, the extending direction of the unit-electrode U1 is the same (second direction), and the orientation directions in these liquid crystal layers CLX are orthogonal to each other. Further, the liquid crystal elements LX1 and LX2, in which the unit-electrode U1 extends in the first direction, are controlled by the first control unit AX1 so that the potential distribution within the liquid crystal layer CLX is the same. This generates a convex or concave linear Fresnel lens-like refractive index distribution having a focal axis in the first direction. Similarly, the liquid crystal elements LX3 and LX4 whose unit-electrodes U2 extend in the second direction are controlled by the second control unit AX2 so that the potential distributions within the liquid crystal layer CLX are the same. This generates a convex or concave linear Fresnel lens-like refractive index distribution having a focal axis in the second direction.

In modification 6, the first direction and the second direction are approximately orthogonal, and the variable focus lens LN in which the liquid crystal elements LX1 to LX4 are arranged in a superimposed manner generates a Fresnel lens-like refractive index distribution with equal annular width. The position of the optical axis can be variously changed in the first direction and the second direction (that is, in the vertical direction and the horizontal direction with respect to the wearer) by controlling the first control section AX1 and the second control section AX2. Note that the first control unit AX1 controls the position of the optical axis of the variable focus lens LN to change in the horizontal direction (X direction), and the second control unit AX2 controls the position of the optical axis of the variable focus lens LN in the vertical direction (Y direction).

Hereinafter, regarding the variable focus eyeglasses 10 of modification 6, the points that are different from the variable focus eyeglasses 10 etc. of the first embodiment will be further explained, and the explanation of the points that are the same as the case of the first embodiment etc. will be omitted as appropriate.

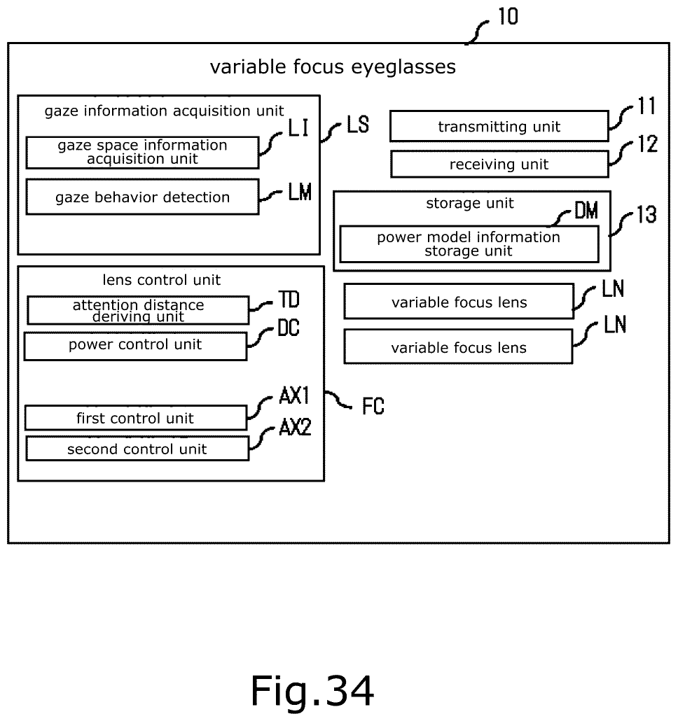

FIG. 34 is a diagram for explaining the functional configuration of variable focus eyeglasses 10 according to modification 6. The variable focus eyeglasses 10 of modification 6 includes a configuration that are a gaze information acquisition unit LS, and a lens control unit FC, and the like. Furthermore, the gaze information acquisition unit LS includes a gaze spatial information acquisition unit LI and a gaze behavior detection unit LM, and the lens control unit FC includes an attention distance deriving unit TD, a power control unit DC, a first control unit AX1, and a second control section AX2.

The attention distance deriving unit TD of the lens control unit FC based on the information regarding the space covered by the user's line of sight acquired by the gaze space information acquisition unit LI and the information regarding the gaze behavior detected by the gaze behavior detection unit LM, derive the distance to the object of interest of the wearer. Further, the power control unit DC refers to the power model information held in the power model information storage unit DM and determines the power to be set for the two variable focus lenses LN. The first control unit AX1 and the second control unit AX2 individually set control voltages to be input to each of the first electrodes E1 of each unit-electrode U1 and each of the second electrodes E2 of each unit-electrode U1. This causes the liquid crystal elements LX1 to LX4 to generate a refractive index distribution corresponding to the power determined by the power control unit DC. In particular, the first control unit AX1 and the second control unit AX2 perform control to change the position of the optical axis of the refractive index distribution based on the wearer's gaze direction detected by the gaze behavior detection unit LM. More specifically, the first and second control units AX1 and AX2 perform control to change the position of the optical axis of the refractive index distribution, which is a distribution corresponding to the power determined by the power control unit DC. (The first control unit AX1 and the second control unit AX2 are present in each of the left and right variable focus lenses LN, but the notation to that effect in FIG. 34 is omitted.)

The position of the optical axis moves based on the wearer's gaze information regarding the wearer's gaze (specifically: the position of the optical axis moves to a position corresponding to the direction of the gaze based on the gaze behavior), making the variable focus eyeglasses 10 comfortable for the wearer.

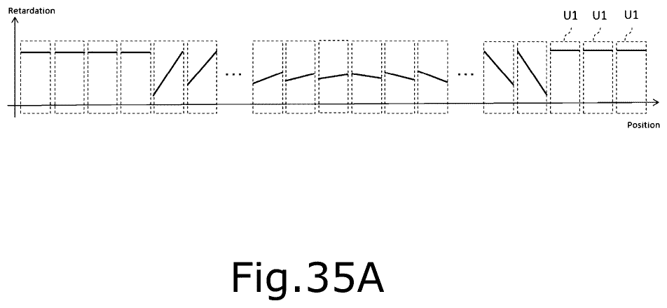

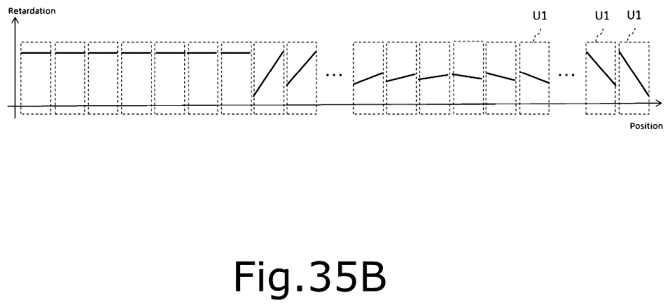

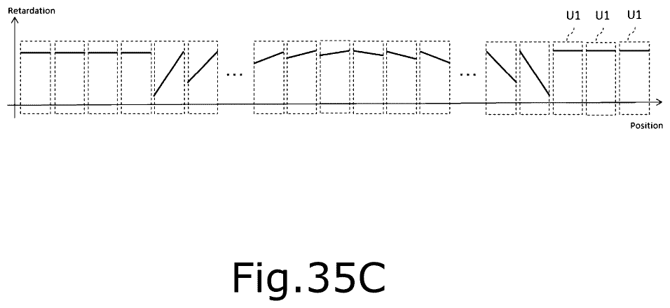

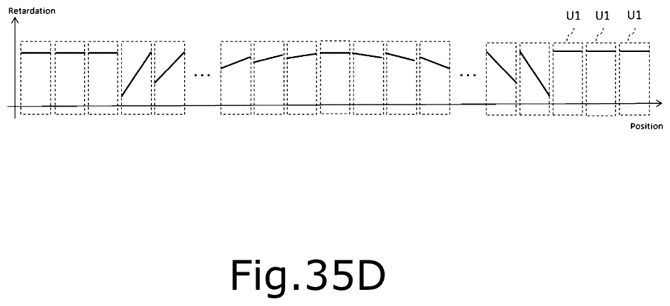

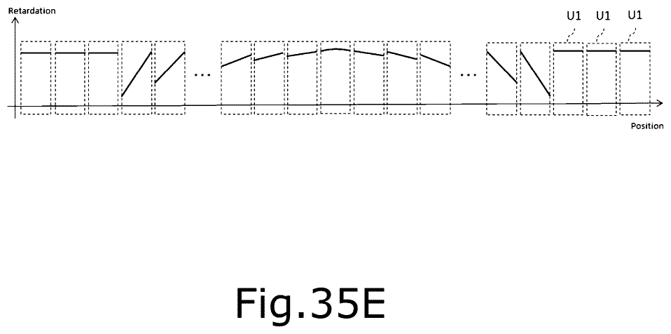

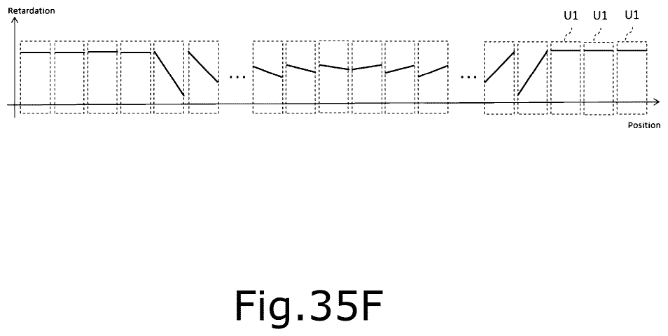

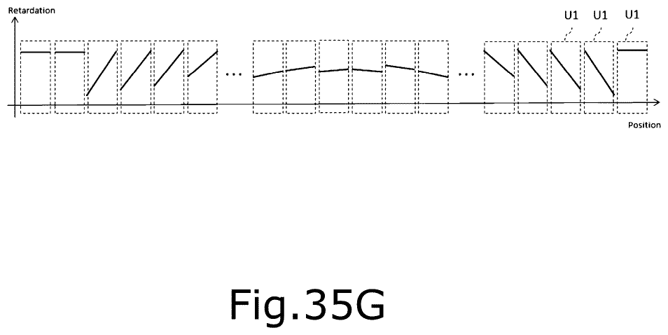

FIG. 35A to 35G are diagrams for explaining how a linear Fresnel lens-like refractive index distribution is generated in the liquid crystal element LX1. As shown in the figure, in the liquid crystal element LX1, control voltages input to each unit-electrode U1 are individually set to generate a refractive index distribution. (Specifically: the first control unit AX1 sets 400 inputs in order to input two types of inputs, the first electrode E1 and the second electrode E2, to each of the 200 unit-electrode U1.). In each figure, the horizontal coordinate is the direction (X direction) in which the unit-electrode U1 is successively arranged, and is the direction perpendicular to the focal axis generated in the liquid crystal element LX1. The broken line frame corresponds to the unit-electrode U1 that are successively installed, and the vertical coordinate indicates the retardation value. In each unit-electrode U1, a smooth retardation gradient can be generated by the control voltage input to the first electrode E1 and the second electrode E2. Note that the description of the retardation value in each figure of FIG. 35 is omitted for the boundary portion between the unit-electrodes U1 corresponding to the gap between the broken line frames.

The linear Fresnel lens-like refractive index distribution in each figure in FIG. 35 includes a plurality of retardation gradients arranged approximately symmetrically as a standard the optical axis. Each of the plurality of retardation gradients has a top where the retardation value becomes high and a bottom where the retardation value becomes low, and the retardation value decreases from the top to the bottom.

FIG. 35A shows a case where the first control section AX1 performs drive control so that the optical axis is located approximately at the center of the arrangement area of the unit-electrode U1, and FIG. 35B shows a case where the first control section AX1 performs drive control so that the optical axis is located on the right side in the figure. As shown in the two figures, the position of the lens section that generates a linear Fresnel lens-like refractive index distribution is controlled by the first control section AX1. The retardation gradient in the unit-electrode U1 depends on the voltage applied to the first electrode E1 and the second electrode E2 and the difference between the voltages applied thereto (the difference in effective value of the control voltage). In the unit-electrode U1 where different control voltages are applied to the first electrode E1 and the second electrode E2, liquid crystal molecules rise as the potential changes from the low potential side to the high potential side (see FIG. 5), and the retardation value decreases.

Further, both ends in FIG. 35A and the left side in FIG. 35B are non-lens portions that do not have a lens function. In the unit-electrode U1 belonging to the non-lens portion, the control voltage of the first electrode E1 and the second electrode E2 is 0V, the retardation value is the upper limit value, and it is in a state of no retardation gradient is generated. In the liquid crystal layer CLX, even when no voltage is applied, the long axis of the liquid crystal molecules is substantially parallel to the transparent substrate CX1, and the retardation value takes the largest value (upper limit). Further, when the long axis of the liquid crystal molecules is perpendicular to the transparent substrate CX1 (the state where the liquid crystal molecules stand up the most), the retardation value takes the smallest value (lower limit value).

Further, as shown in FIGS. 35A and 35B, in a region where a linear Fresnel lens-like refractive index distribution occurs, the retardation gradient becomes steeper as the distance from the optical axis increases. Therefore, the retardation gradient at the position farthest from the optical axis has the maximum gradient amount (the amount of change in the retardation value per unit length in the X direction). Furthermore, the maximum value of the retardation gradient amount in modification 6 is determined, in general rule, depending on the width of the region AR (see FIG. 5) and the thickness of the liquid crystal layer CLX. Therefore, by narrowing the width of the unit-electrode U1 and increasing the thickness of the liquid crystal layer CLX, it is possible to further steepen the gradient of retardation that can occur in each unit-electrode U1 of the liquid crystal element LX.

Next, the retardation gradient formed by the unit-electrode U1 will be further explained using FIGS. 35A to 35C. FIGS. 35A and 35B show several retardation gradients located symmetrically about the optical axis, with the top of the gradient being lower than the upper retardation limit and the bottom being higher than the lower retardation limit. In contrast, in FIG. 35C, the tops of several retardation gradients located symmetrically about the optical axis are the same as the retardation upper limit.

In a liquid crystal material such as the liquid crystal layer CLX, when a voltage higher than a threshold voltage is applied, liquid crystal molecules begin to rise from a parallel state. (At a voltage lower than the threshold voltage, the liquid crystal molecules become parallel to the transparent substrate CX1, and the retardation value remains at the upper limit and does not change.). When delicately controlling the amount of retardation gradient, it is easier to perform stable control at a voltage higher than the threshold voltage than near the threshold voltage where the direction of liquid crystal molecules tends to change rapidly. Further, near the threshold voltage, the influence of disturbance on the retardation gradient tends to be large.

Therefore, as shown in FIGS. 35A and 35B, in the lens portion that generates a linear Fresnel lens-like refractive index distribution, the top of the retardation gradient can be preferable to control as follows in at least a portion of the unit-electrode U1. That is, for example, it is preferable to control so that the top of the retardation gradient in some unit-electrodes U1 arranged at the center of the lens section has a retardation value lower than the upper limit of the retardation of the liquid crystal layer CLX. Specifically, control is performed so that a voltage having an effective value larger than a threshold voltage at room temperature (for example, 20° C.) is input to the lower potential side electrode of the first electrode E1 and the second electrode E2. At the top of the retardation gradient, a voltage higher than the threshold voltage may be input to the electrode on the low potential side so that the voltage is lower than the upper limit value. For example, the top of the gradient may be lower than the upper limit by 10% or more. 20% or more, or 30% or more of the difference between the upper and lower retardation values.

Further, when the amount of retardation gradient is less than or equal a predetermined standard (for example, when it is less than or equal ½ of the maximum value of the retardation gradient amount), the retardation value at the top may be lower than the upper limit value. In the unit-electrode U1 that generates a retardation gradient with a small gradient amount, by setting the retardation value at the top to be lower than the upper limit value, stable control can be performed.

Note that power can be saved by making the top of the retardation gradient equal to the retardation upper limit value in at least a portion of the unit-electrodes U1 of the lens portion as shown in FIG. 35C. Specifically, in at least a part of the unit-electrode U1, by setting the effective value of the control voltage to the low potential side electrode of the first electrode E1 and the second electrode E2 to be less than or equal the threshold voltage (or 0V), power saving can be achieved. Note that at least some of the unit-electrodes U1 described above include the unit-electrode U1 located at the end of the lens portion having a lens function (located at the boundary with the non-lens portion), and at least a part of the unit-electrode U1 other than the unit-electrode U1 in the lens portion. Furthermore, when the amount of retardation gradient exceeds a predetermined standard (for example, when it becomes ½ or more of the maximum value of the amount of retardation gradient), power saving may be achieved by controlling the upper limit of the retardation gradient to be the same as the upper limit value of the retardation.

Note that the threshold voltage Vth of the liquid crystal material is expressed by the following equation (2). K33 (bend elastic constant) is used for Keff, ε0 is the dielectric constant in vacuum, and Axis the dielectric constant anisotropy. For K33 and Δε, values according to the specifications of the liquid crystal material may be adopted.

$$\begin{matrix}[ Math 2 ] & \\ {V}_{t h} = π \sqrt{{K}_{eff} / {ε}_{0} Δ ε} & ( 2 )\end{matrix}$$

Note that the retardation value at the unit-electrode U1 can be measured using a micro-area polarization analyzer (OPTIPRO micro) manufactured by Shintech, and the wavelength when measuring the retardation value is 550 nm and the spot diameter is φ3 μm. Note that retardation (phase delay) is expressed as Δn×d. The symbol Δn is the refractive index anisotropy that the liquid crystal layer CLX has, and the symbol d corresponds to the thickness of the liquid crystal layer CLX.

Next, the linear Fresnel lens-like refractive index distribution formed by the plurality of unit-electrodes U1 will be further explained using FIGS. 35A, D, and E. First, in FIG. 35A, the optical axis is located in a narrow area between two unit-electrodes U1. In FIG. 35D, the optical axis of the linear Fresnel lens-like refractive index distribution is located at the unit-electrode U1 that generates a flat retardation distribution with no gradient. In the unit-electrode U1 where the optical axis is located in FIG. 35D, the same control voltage equal to or lower than the threshold voltage is input to the first electrode E1 and the second electrode E2.

In FIG. 35D, it is considered that an ideal lens-like refractive index distribution is formed across the three central unit-electrodes U1, but the central unit-electrode U1 where the optical axis is located does not have any curvature. Since it does not function as a lens, it does not contribute to improving the image quality of the lens. Therefore, as shown in FIG. 35A, it is preferable for the optical axis to be located between the two unit-electrodes U1, since this results in a refractive index distribution with high lens image quality:

In FIG. 35E, in the unit-electrode U1 where the optical axis is located at the center, curvature is generated by inputting the same control voltage slightly higher than the threshold voltage to the first electrode E1 and the second electrode E2. When the control voltage input to the first electrode E1 and the second electrode E2 is relatively low, the liquid crystal molecules in the portion near the first electrode E1 and the second electrode E2 rise slightly. However, in the central portion away from the first electrode E1 and the second electrode E2, the direction of the liquid crystal molecules becomes parallel to the transparent substrate CX1, and the retardation value tends to transition at the upper limit value. As shown in FIG. 35(E), applying the following control voltage to the unit-electrode U1 contributes to improving the lens image quality: This control voltage is a control voltage that allows the retardation value at the center of the unit-electrode U1 corresponding to the optical axis to take a larger value than the retardation values at the first electrode E1 and the second electrode E2 (the retardation value at the center is approximately the upper limit value).

FIG. 35F shows how a concave lens-type linear Fresnel lens-like refractive index distribution is generated. In the case of a concave lens type, it is difficult to control the unit-electrode U1 to generate curvature at a position corresponding to the optical axis as shown in FIG. 35E. Therefore, as in the case of FIG. 35A, it is preferable to perform control so that the optical axis is located between two adjacent unit electrodes U1.

FIG. 35G is a diagram showing how a linear Fresnel lens-like refractive index distribution is generated in the liquid crystal element LX1 when the lens power is reduced compared to the case of FIG. 35A. Since the liquid crystal element LX1 is limited by the width of the unit-electrode U1 and the thickness of the liquid crystal layer CLX, if the lens power is increased, the width of the lens portion must be narrowed. On the other hand, when the lens power is decreased as shown in FIG. 35G, the width of the lens portion can be expanded, and the diameter of the Fresnel lens that can control the position of the optical axis by the variable focus lens LN can be increased. Further, as in the vicinity of the optical axis in FIG. 35G, a retardation gradient may be formed by connecting two or more unit-electrodes U1.

In FIG. 35B, the optical axis of the linear Fresnel lens-like refractive index distribution moves to the right, and the retardation gradient of the unit-electrode U1 at the right end of the liquid crystal element LX1 becomes maximum, resulting in a distribution that is symmetrical about the optical axis. Furthermore, the optical axis may be moved to the right to generate a linear Fresnel lens-like refractive index distribution in which the number of phase difference gradients on the right side is smaller than the number of phase difference gradients on the left side.

Note that each of the first control unit AX1 and the second control unit AX2 may be configured to include a first drive circuit that outputs an input voltage to each of the plurality of first electrodes E1, and a second drive circuit that outputs an input voltage to each of the plurality of second electrodes E2. The refractive index distribution of a linear Fresnel lens as shown in FIG. 35A etc. has the following state. That is, the input to the first electrode E1 of the n-th unit-electrode U1 on the right side with respect to the optical axis, and the input to the second electrode E2 of the n-th unit-electrode U1 on the left side with respect to the optical axis, have a common state. Therefore, by controlling the first electrode E1 and the second electrode E2 with separate drive circuits, a simple circuit configuration can be achieved.

As seen in FIG. 35A etc., in modification 6, the retardation value is controlled to be zero in the unit-electrode U1 of the non-lens part located outside the lens part that generates a linear Fresnel lens-like refractive index distribution. However, it may be set to a constant value larger than zero, and is not particularly limited. Furthermore, by performing control to reduce image quality (control to scatter incident light) in the non-lens portion, it is possible to reduce the visibility of unnecessary scenery for the wearer and improve usability: The image quality may be made to deteriorate by inputting the following control voltages to the first electrode E1 and the second electrode E2 in the unit-electrode U1 of the non-lens portion. Specifically: this control voltage is a voltage with different phases, or a voltage such that the frequency on the high potential side is lower than the frequency on the low potential side. Further, for example, the image quality may be made to deteriorate by forming a random retardation gradient in each unit-electrode U1 of the non-lens portion.

Furthermore, as the variable focus lens LN, astigmatism may be generated by changing the focal length determined by the first control section AX1 and the focal length determined by the second control section AX2. Furthermore, as in modification 5, six liquid crystal elements are stacked and arranged so that the extending direction of the unit-electrode U1 is approximately 60 degrees, so that the amount of astigmatism and the axial direction may be possible to control. In this case, the focal axes of the linear Fresnel lens-like refractive index distribution formed by a plurality of liquid crystal elements arranged at an angle of 60 degrees are controlled so that they intersect at one point.

Note that in the first control unit AX1 and the second control unit AX2, the frequencies of the control voltages input to each of the first electrodes E1 and each of the second electrodes E2 may be the same, or may be different frequencies. Further, the frequency may be different for each unit-electrode U1, or any one of about 3 to 10 different frequencies may be used for input to the 200 unit-electrodes U1.

Further, in the liquid crystal elements LX1 to LX4, the width of the unit-electrode U1 is not particularly limited, and may be, for example, 70 μm or more and 400 μm or less, and preferably 100 μm or more and 300 μm or less. Further, in the variable focus lens LN, the region in which a refractive index distribution can occur does not necessarily have to be square. For example, the vertical and horizontal sizes of the liquid crystal elements LX1 to LX4 may be determined so that they are rectangular.

Further for example, in liquid crystal elements LX1. LX2 in which unit-electrodes U1 extend in the first direction, and liquid crystal elements LX3. LX4 in which unit-electrodes U1 extend in the second direction, the extending length in which the unit-electrode U1 and arrangement pitch may be changed.

Further, the liquid crystal elements LX1 and LX2 may function as variable prisms that can control the deflection angle of incident light by controlling the retardation gradients occurring in each unit-electrode U1 to the same gradient by the first control unit AX1 (In this case, the focus becomes infinity and the lens function is lost.). Similarly: the liquid crystal elements LX3 and LX4 may also function as variable prisms. Therefore, the lens control unit FC of the variable focus eyeglasses 10 may have a lens/prism switching section so as to may be able to perform control for switching between the lens function and the prism function.

### [About Modification 7 of Variable Focus Eyeglasses]

Next, a seventh modification of the variable focus eyeglasses 10 that can be applied to the eyeglass system 1 such as the first embodiment will be described. Below, the points that are different from the variable focus eyeglasses 10 of the first embodiment will be mainly explained, and the explanation of the points that are almost the same as the first embodiment and the like will be omitted as appropriate.

### <1. Component Composition>

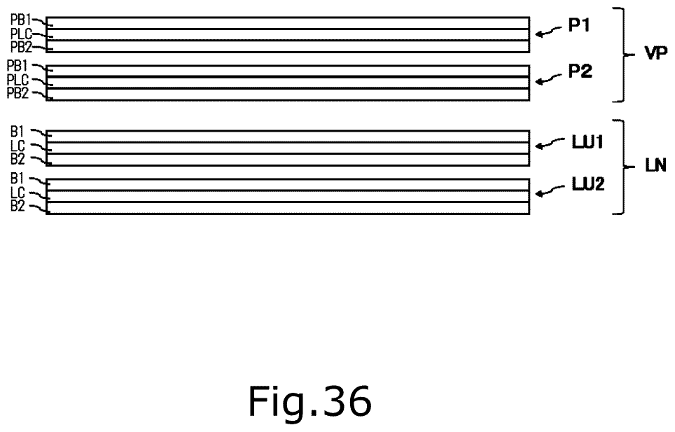

FIG. 36 is a diagram for explaining the component structure of the lens portion of the variable focus eyeglasses 10 of modification 7, which is configured to include a variable focus lens LN and a variable prism VP. Like the variable focus lens LN of the first embodiment, the variable focus lens LN is configured to include two liquid crystal elements LU1 and LU2 whose alignment directions are orthogonal to each other. The variable prism VP is configured to include two liquid crystal prism elements P1 and P2 whose alignment directions are perpendicular to each other.

The liquid crystal prism elements P1 and P2 are configured to include two transparent substrates PB1 and PB2 that sandwich a liquid crystal layer PLC. In the liquid crystal prism elements P1 and P2, like the liquid crystal element LX1 of the sixth modification, a plurality of unit-electrode U1 formed in a straight line and having substantially the same width are arranged in series. However, this is different from the case of modification 6 in that each unit-electrode U1 always generates substantially the same retardation gradient. Specifically; each of the first electrodes E1 of each unit-electrode U1 is connected with one lead wire and the first voltage is inputted, and each of the second electrodes E2 of each unit-electrode U1 is connected with another one and the second voltage inputted.

Further, the extending direction of the unit-electrodes U1 of the liquid crystal prism elements P1 and P2 is in the vertical direction with respect to the wearer of the variable focus eyeglasses 10, and the variable prism VP deflects horizontal direction the gaze of the wearer of the variable focus eyeglasses 10.

### <2. Deflection Angle Control Using Variable Prism>

Generally, it is known that there are people who have strabismus, and it is known that there are also people who have symptoms such as convergence insufficiency, convergence excess, divergence insufficiency, and divergence excess. Furthermore, it is known that people with convergence insufficiency are prone to eye strain, and asthenopia may cause the convergence force to weaken, resulting in convergence insufficiency: The variable focus eyeglasses 10 of modification 7 provide a comfortable feeling of use by controlling the deflection angle by the variable prism VP to address the above-mentioned problems that may occur with binocular vision.

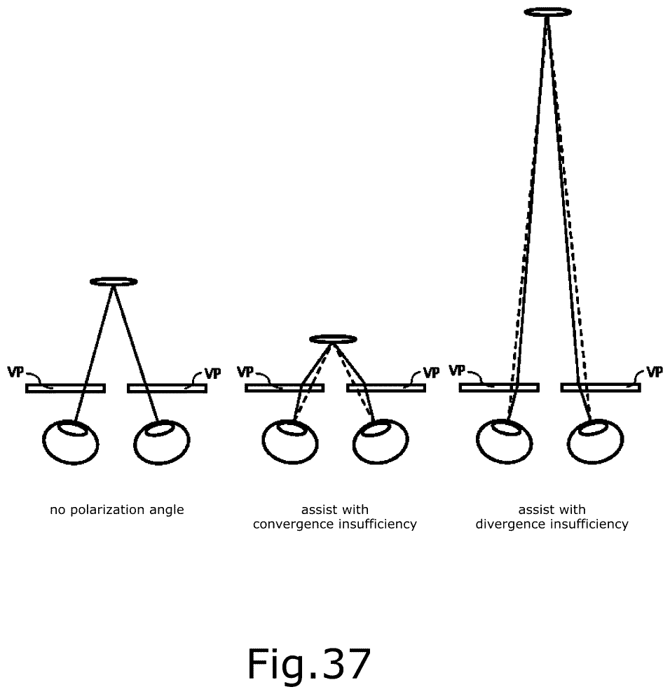

FIG. 37 is a diagram illustrating how the deflection angle by the variable prism VP is controlled to alleviate problems occurring in the binocular vision function, such as convergence insufficiency and divergence insufficiency. In the figure, from the left side, the case of “no deflection angle”, the case of “assistance of convergence insufficiency”, and the case of “assistance of divergence insufficiency” will be described.

In the case of “no deflection angle”, the variable prism VP does not function, and the wearer's gaze passes through the prism as it is, and gazes at the object.

On the other hand, in the case of “convergence insufficiency assistance”, the variable prism VP compensates by refracting the wearer's gaze inward (deflect in the convergence direction) (solid line). That is, when the object of gaze is nearby; people with convergence insufficiency have difficulty seeing cross-eyed due to decreased eye muscle strength or fatigue, resulting in problems with binocular vision, and therefore, the variable prism VP operates as described above. In addition, in the case of “divergence insufficiency assistance”, the variable prism VP compensates by refracting the wearer's gaze outward (deflect in the divergent direction) (solid line). That is, when the object of gaze is far away; by divergence insufficiency, resulting in problems with binocular vision, and therefore, the variable prism VP operates as described above.

### <3. Functional Configuration>

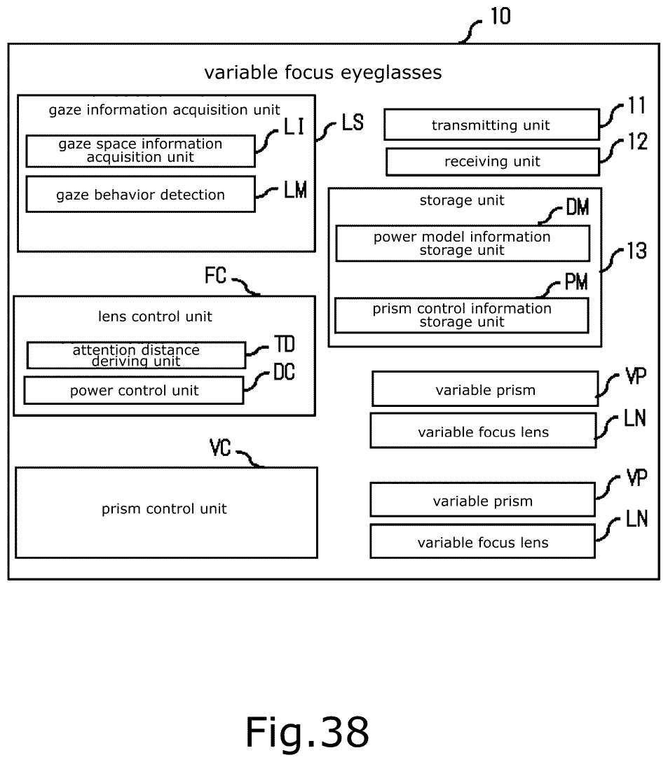

FIG. 38 is an explanatory diagram of the functional configuration of variable focus eyeglasses 10 of modification 7. As shown in the figure, the variable focus eyeglasses 10 include a pair of variable prisms VP and a variable focus lens LN, and further include a gaze information acquisition section LS, a lens control section FC, a prism control section VC, and a storage unit 13. In addition to the power model information storage unit DM, the storage unit 13 is configured to include a prism control information storage unit PM.

As in the case of the fifth embodiment, the variable focus lens LN is controlled to generate a refractive index distribution corresponding to the power determined based on the information acquired by the gaze information acquisition unit LS and the power model information.

In particular, the deflection angle of the variable prism VP is controlled based on the information acquired by the gaze information acquisition unit LS and the prism control information stored in the prism control information storage unit PM.

The prism control information is information for controlling the deflection angle of the variable prism VP according to the behavior and situation of the wearer. Specifically, the information indicates the relationship between the distance to the object that the wearer is gazing at and the deflection angle of the variable prism VP that the wearer should set, but is not particularly limited.

Prism control information is input in advance by the wearer and stored in the prism control information storage unit PM. Specifically, in the case of a person who has a problem of a convergence insufficiency problem, prism control information that generates a deflection angle that refracts the gaze inward during near vision is input in advance. Additionally, in the case of a person who has a problem of a divergence insufficiency problem, prism control information that generates a deflecting angle that refracts the gaze outward during far vision in input in advance.

Below, using FIG. 38, the functional configuration of the variable focus eyeglasses 10 of modification 7, control by the prism control unit VC, etc. will be described in more detail.

In the variable focus glasses 10 of modification 7, a LiDAR sensor is arranged as the gaze spatial information acquisition unit LI, as in the case of the fifth embodiment.

Further, on the rim 101 serving as the outer frame of the variable focus lens LN, a light source of near-infrared light and an image sensor that acquires reflected light from the cornea are arranged at a plurality of locations as the gaze behavior detection unit LM.

The prism control unit VC controls the two variable prisms VP to generate deflection angles based on the distance to the object that the wearer is paying attention to, derived by the attention distance derivation unit TD, and the prism control information. As in the case of the fifth embodiment, the distance to the object of interest is derived based on the gaze spatial information acquired by LiDAR and at least one gaze direction acquired by the gaze behavior detection unit LM. In other words, the three-dimensional point cloud information acquired by LiDAR includes point cloud information of the object that the wearer is paying attention to. Therefore, the distance to the object of interest can be derived by specifying the point cloud information of the object of interest based on one gaze direction by the wearer.

Note that the prism control information may indicate the relationship between the convergence angle (or pupil size) of the wearer and the deflection angle to be set by the wearer. In this case, the gaze behavior detection unit LM detects the gaze direction of both eyes, and controls the deflection angle based on the convergence angle based on the direction. Alternatively, detect the pupil size (thereby derive the distance to the target object), and thereby control the deflection angle.

Further, the prism control unit VC may include an eye fatigue information acquisition unit that outputs eye fatigue information regarding the degree of eye fatigue. The degree of eye fatigue can be estimated by, for example, measuring blinks or detecting the reaction of the pupils to light. As for the former, eye fatigue is estimated based on whether the wearer's blinks are detected more often than usual by the gaze behavior detection unit LM. Regarding the latter, for example, the degree of eye fatigue is estimated by installing a sensor that detects optical stimulation in the variable focus eyeglasses 10 and measuring the pupil constriction rate, contraction speed, etc. immediately after detecting the optical stimulation. The eye fatigue information acquisition unit outputs eye fatigue information based on these estimation results. The prism control unit VC may control the deflection angle as follows based on the eye fatigue information. That is, the prism control unit VC may generate a deflection angle to deflect the wearer's gaze inward when the wearer suffers from eye fatigue and performs near vision.

Note that when the prism control unit VC includes an eye fatigue information acquisition unit, the prism control information may also take eye fatigue into consideration.

Therefore, for example, the prism control information may be information indicating the relationship between the deflection angle to be set for the variable prism VP and eye fatigue of the wearer. Furthermore, the information may be information indicating the relationship between the deflection angle to be set, eye fatigue, and the distance to the object of interest (or convergence angle or pupil size).

Note that the gaze spatial information acquisition unit LI may be configured with a laser ranging module as in the first embodiment. In this case, the deflection angle may also be controlled by the prism control unit VC when the distance to the detected object becomes farther or closer than a certain reference.

Note that the prism control information in the variable focus eyeglasses 10 of modification 7 may be information in which the deflection angle is set so as to compensate for the wearer's oblique, depending on the wearer's behavior and situation.

Note that the variable prism VP of the variable focus eyeglasses 10 of modification 7 may be configured to include a liquid crystal prism configured by a unit-electrode U1 extending in the left-right direction with respect to the wearer. Thereby, the variable prism VP may allow the wearer to change the gaze in the vertical and horizontal directions.

### <4. Head Mounted Display>

In the following, a case will be described in which the variable focus eyeglasses 10 of modification 7 are a head-mounted display that is a glasses-type information device that includes an image display device that covers the wearer's eyes.

It is known that so-called “VR sickness” occurs with head-mounted displays. “VR sickness” is thought to be caused partly by convergence accommodation inconsistencies, but by applying variable prism VP to head-mounted displays and dealing with binocular vision problems such as convergence insufficiency mentioned above, it is thought that VR sickness can be alleviated.

Variable focus eyeglasses 10 (hereinafter simply referred to as HMD), which are head-mounted displays, generate a visual field image representing a visual field from the wearer's viewpoint based on virtual (or real) three-dimensional spatial data, and display the visual field image on the display device in front of the user's eyes. The HMD of modification 7 has the means for executing control for estimating an object to which the wearer pays attention within the visual field image. This means executes the above control based on the direction of the line of sight detected by the gaze behavior detection unit LM which acquires an eyeball image using near-infrared rays.

Furthermore, the HMD of modification 7 derives information regarding the position of the object to which the wearer pays attention (relative position to the wearer) based on the three-dimensional spatial data from which the visual field image is generated. The prism control unit VC controls the deflection angle based on information regarding the position (by deriving the distance from the wearer). Furthermore, the HMD of modification 7 includes means for deriving information regarding the position of the object that the wearer is paying attention to (relative position with respect to the wearer) based on the three-dimensional spatial data from which the visual field image is generated. The prism control unit VC controls the deflection angle based on the derived position information (by deriving the distance to the wearer).

By controlling the deflection angle of the variable prism VP according to the distance (positional relationship) between the wearer and the object the wearer is gazing at, problems such as lack of convergence and lack of divergence that occur with binocular vision function can be alleviated.

Also, when the wearer attempts to gaze at a position far from (or close to) the screen with reference to the optical position of the screen where the visual field image is displayed, the deflection angle of the variable prism VP may be generated.

Note that it is desirable that the attention distance deriving unit TD is capable of accurately deriving the distance to the object of interest even when there is eye fatigue. Therefore, for example, the position of the object of interest can be estimated based on the gaze space information (field image in the case of HMD) acquired by LiDAR and the gaze direction of one eye (or the gaze direction of both eyes) acquired by the gaze behavior detection unit LM, and it is preferable that the distance to the object can be derived based on this estimated position information. From the positional relationship between the object of interest and the wearer (specifically: the distance to the object of gaze), the gaze direction of both eyes of the wearer can be geometrically derived. When there is a difference between the gaze direction of both eyes derived from the positional relationship and the gaze direction of both eyes detected by the gaze behavior detection unit LM due to circumstances such as eye fatigue, the prism control unit VC may control the deflection angle based on this difference. This is thought to alleviate eye fatigue.

Note that the storage unit 13 may store prism control information indicating the relationship between the distance to the object of interest and the convergence angle of the wearer. This prism control information is information indicating the above relationship in a state without eye strain. Then, if there is a difference between the convergence angle according to the distance to the gaze target estimated by the attention distance derivation unit TD and the convergence angle according to the gaze direction of both eyes detected by the gaze behavior detection unit LM, eye fatigue is thought to be alleviated by the prism control unit VC controlling the deflection angle based on the difference.

Note that even if the variable focus eyeglasses 10 of modification 7 is applied to the eyeglass system 1 of the fifth embodiment etc., and the prism control information is stored in the storage unit of the server device 30 in the same way as the power model information. Further, the variable focus eyeglasses 10 in this case may include an update control section that accepts updates of prism control information based on user input, etc., and the updated prism control information may be stored in the server device 30. In addition, the prism control information accumulated in the server device 30 may be used to construct learning data for generating a predictive diagnosis model and a change prediction model (see FIGS. 27A and 27B) in the same manner as the power model information.

### [Others]

Note that the power setting information in the first embodiment and the like is configured to include six types of information: “SPH”, “CLY”, and “AXIS” for both the left and right eyes, but the present invention is not limited to this aspect, and may include information regarding further power setting, or may be configured by one or two types of parameters, which are fewer than six types. Further, the power setting information corresponding to the sp multifocal mode or the area-divided multifocal mode may include further information other than the above six types.

Note that in a plurality of unit-electrodes U1 such as the liquid crystal element LU1, an insulating wall-like structure is arranged at the boundary between two adjacent unit-electrodes U1 using an ultraviolet curing resin to may partition the liquid crystal layer LC. This improves the strength when bending the liquid crystal element LU1 and the like. Furthermore, in the liquid crystal element LF1, the liquid crystal prism element P1, etc. in which a plurality of linear unit-electrodes U1 are arranged, a wall-like structure may be similarly arranged at the boundary between two unit-electrodes U1, or a wall wall-like structure may be disposed that extends in a direction intersecting the unit-electrode U1 and overlaps the resistance layer HR. The former improves the strength against bending in a direction perpendicular to the extending direction of the unit-electrode U1, and the latter improves the strength against bending in a direction parallel to the extending direction of the unit-electrode U1. Alternatively, both the former and latter wall-like structures may be arranged, and the wall-like structures may be formed in a mesh shape. Note that it is preferable that the extending direction of the latter wall-like structure forms an angle of 45 degrees or more (or an angle of 90 degrees) with the extending direction of the unit-electrode U1, and that it has a thin line shape. Furthermore, the former and latter wall-like structures do not necessarily need to be formed of ultraviolet curing resin.

Although the diagnostic information in the fifth embodiment and the like is acquired by transmission from the medical worker client device 40, it is not necessarily limited to this. Therefore, for example, in the case of the fifth embodiment, a medical worker who has checked the power model information up to the present time and other information may create diagnostic information by adding a label indicating the possibility of a specific disease.

Note that although the variable focus eyeglasses 10 and the HMD of modification 7 are equipped with both the variable prism VP and the variable focus lens LN, the eyeglasses may be equipped with only the variable prism VP (variable prism eyeglasses), or may be an HMD or smart glasses that includes only a prism VP.

Note that in the variable focus lens LN of the first embodiment and each modification, a pair of liquid crystal elements whose alignment directions are orthogonal are overlapped. However, for example, in the case of a glasses-type information device in which an image display device using a liquid crystal display with uniform polarization directions is placed in front of the eyes, it may be a variable focus lens LN including a liquid crystal element LU1 or the like having one type of orientation direction corresponding to the polarization direction.

Note that the power model information in the variable focus eyeglasses 10 such as the first embodiment described above is for controlling the power setting information corresponding to a plurality of modes to be switched depending on the gaze related information. For example, the power model information in the fifth embodiment is information for controlling the refractive index distribution of the variable focus lens LN so that it can be switched depending on the distance to the object of interest, as described in FIG. 24. However, the power model information is not necessarily limited to this manner. Therefore, the power model information may be information for controlling to change the refractive index distribution of the variable focus lens LN, for example, the information may be information for controlling to change depending on whether the user behavior corresponds to a case in which the gaze is directed upward or downward.

Further, in the first embodiment and the like, the power setting switching unit SW controls the mode to change between the first to third modes, but the mode is not necessarily limited to this mode. That is, the control may be performed to change between two modes according to the gaze related information, or may be controlled to change between four or more modes. Further, the plurality of modes switched by the power setting switching unit SW may include a state where there is no power setting (a state where “SPH” and “CLY” are zero).

Note that in the second embodiment and the third embodiment, the lifestyle information shown in FIGS. 16 and 17 includes gaze-related information and control content information. However, this information may or may not be used as learning data for generating the predictive diagnosis model in the second embodiment and as input data for learning data for generating the power setting prediction model in the third embodiment.

Note that the server device 30 of the first to third embodiments may include an optometry information storage section 34g similar to that of the fourth embodiment, and optometry information may be stored in the optometry information storage section 34g. In this case, for example, the training data included in the learning data-sets of the predictive diagnostic models and frequency setting predictive models (see FIGS. 12 and 18) of the first to third embodiments may remain as they were in the original embodiments, and at the same time, the input data in the learning data may include optometry information (some optometry information acquired before the acquisition timing of the teacher data). Furthermore, the input data for estimation by these models may be configured to include optometry information, and in this way: the accuracy of predictive diagnostic information and power setting predictive information may be improved.

Note that the server device 30 of the third embodiment may include an optometry information storage unit 34g in which optometry information is accumulated, and the teacher data in the learning data-set of the power setting prediction model (see FIG. 18) may be optometry information instead of the power setting information, and the input data in the learning data-set may include the optometry information and power setting information. If there is any relationship between the optometry information and the power setting information, it is considered that information for predicting the power setting can be obtained from the output of the power setting prediction model in such a case.

Note that in the first embodiment and the like described above, data communication between the variable focus eyeglasses 10 and the server device 30 is performed via the operation device 20, but the present invention is not necessarily limited to this mode. For example, data may be directly transmitted and received between the variable focus eyeglasses 10 and the server device 30.

Note that in each unit-electrode U1 of the liquid crystal element LU1 in the first embodiment and the like, an insulating layer IS1 is interposed between the resistance layer HR and the first electrode E1 and the second electrode E2, but this embodiment is not limited to this mode. For example, the first electrode E1 and the second electrode E2 may be formed in contact with the resistance layer HR.

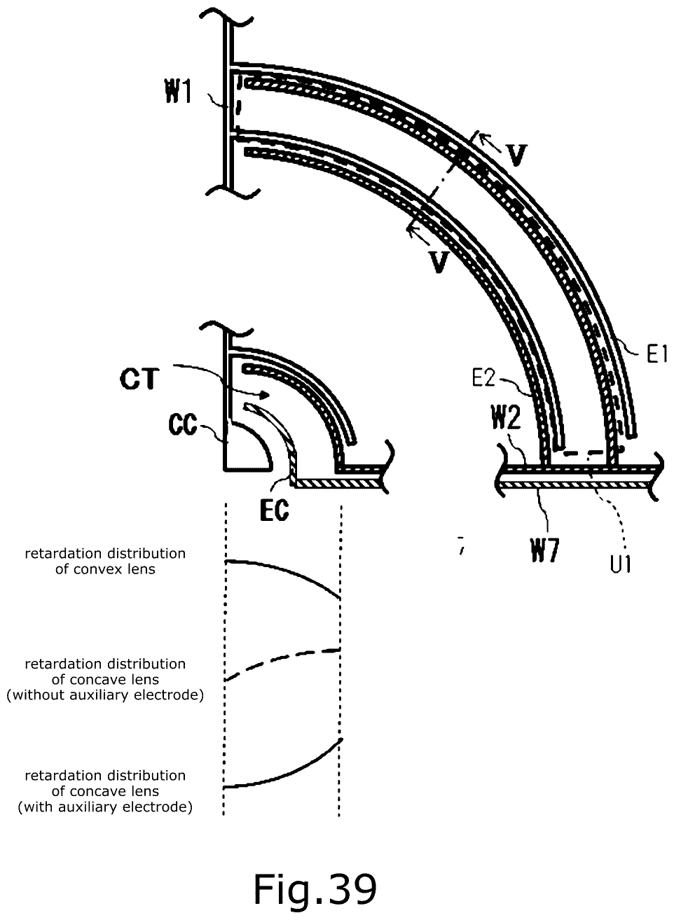

Note that in the liquid crystal elements LU1, LF1, etc. of the first embodiment, etc., although a center electrode CT (the center electrode CT is not shown in FIG. 31A) having a core electrode CC at the center position is arranged, an auxiliary electrode may be arranged in the center electrode CT. FIG. 39 is a diagram showing an example in which the auxiliary electrode EC is arranged on the center electrode CT. As shown in FIG. 39, FIG. 4, and FIG. 31A, the center electrode CT includes a core electrode CC and an arc-shaped (or linear) second electrode E2, and the second electrode E2 is formed in a thin line shape. However, the core electrode CC is formed in a fan shape (or a wide linear shape). In addition, the lower graph in FIG. 39 shows the retardation distribution in each case of “convex lens (no auxiliary electrode)”, “concave lens (no auxiliary electrode)”, and “concave lens (with auxiliary electrode)”, and the vertical axis is in the retardation value, the horizontal axis indicates the radial position in the center electrode CT.

Here, when forming a convex Fresnel lens-like refractive index distribution in the liquid crystal element LU1 etc., even if the auxiliary electrode EC is not arranged, the retardation distribution is formed that becomes convex upward and becomes steep slope as it moves away from the center of the center electrode CT. However, when a concave Fresnel lens-like refractive index distribution is formed and there is no auxiliary electrode, the retardation distribution becomes as shown by the broken line curve in FIG. 39. Therefore, the center electrode CT does not function as a concave lens, and as a result the lens performance will deteriorate. Such a difference in retardation distribution between the unevenness of the center electrode CT is considered to be caused by the asymmetry of the structure of the center electrode CT (see FIG. 6, etc.). Specifically, this is considered to be due to the fact that the width of the core electrode CC is wider than the second electrode E2 and that the space between the core electrode CC and the second electrode E2 is relatively large.

In the example of FIG. 39, place the auxiliary electrode EC the center electrode CT in order to improve the retardation distribution when forming a concave Fresnel lens-like refractive index distribution. Here, the relationship among the core electrode CC, second electrode E2, and auxiliary electrode EC is as follows. That is, let P be the width of the space between the core electrode CC and the second electrode E2, and let P1 be the distance from the end of the core electrode CC on the space side to the center of the auxiliary electrode EC. Furthermore, if the distance to the end of the second electrode of the auxiliary electrode EC on the space side is P2, then “P=P1+P2”. In this case, place the auxiliary electrode EC the side closer to the core electrode CC in the space between the core electrode CC and the second electrode E2. That is, it is preferable that the size of P1 is set to be greater than or equal to P×1/5 and less than or equal to P×1/2.

Further, the effective values of the voltages input to the core electrode CC, the second electrode E2, and the auxiliary electrode EC are assumed to be V1e, V2e, and V3e, respectively. When generating a concave retardation distribution, the voltage V3e of the auxiliary electrode EC is preferably set higher than (V1e−V2e)×P2/(P1+P2)+V2e. Note that V3e in the case of generating a convex retardation distribution may be set to a value equal to or less than (V2e−V1e)×P1/(P1+P2)+V1e, or the auxiliary electrode EC may be made floating.

Further, as shown in FIG. 39, the auxiliary electrode EC is connected to the seventh lead wire W7 and supplied with voltage. In the case of FIG. 39, the center electrode CT has a fan shape with a center angle of 90 degrees, and the liquid crystal element LU1 has four center electrodes CT, and four auxiliary electrodes EC and four lead wires for supplying voltage to each of the auxiliary electrodes are arranged. However, for example, the common input parts C1a to C1d may be configured by one circular common input part C1, and the group of unit-electrodes U1 in the common input section C1 may have an annular shape with a central angle of approximately 360 degrees. Furthermore, the electrode CT may be formed into a circular shape, and the number of auxiliary electrodes EC and lead wires connected thereto may be one each. (Note that the common input parts C2a to C2d and C3a to C3d are the same as in the first embodiment (see FIG. 8A)). In this case, two lead wires connected to the unit-electrode U1 group belonging to the common input section C1 and a lead wire W7 connected to the auxiliary electrode EC are placed side by side. When arranging the auxiliary electrode EC in the center electrode CT in this way: the number of voltage input channels can be reduced by configuring the center with one circular common input part C1, and the duplication of lead wires can be avoided. As a result, the film formation process of the lead wire W7 for the auxiliary electrode EC and the lead wires for the first electrode E1 and the second electrode E2 of each common input section can be made more efficient.

Note that the line width of the auxiliary electrode EC may be made thinner than the line width of the second electrode E2 so as to locally influence the retardation distribution of the center electrode CT. However, it goes without saying that there is no problem even if the line width of the auxiliary electrode EC is equal to or larger than the second electrode E2.

In addition, although the variable focus eyeglasses 10 in the first embodiment etc. have the variable focus lenses LN fixed by two rims 301 as shown in FIG. 2, the variable focus eyeglasses 10 may be applied to goggle type variable focus eyeglasses 10. Furthermore, the liquid crystal element LU1 and the like in each of the embodiments and modifications may be applied to a contact lens, an intraocular lens, or the like.

In the eyeglass system 1 such as the first embodiment, a liquid crystal lens capable of forming a concave-convex Fresnel lens-like refractive index distribution is employed as the variable focus lens LN of the variable focus eyeglasses 10, but it is not necessarily limited to this. Further, the variable focus eyeglasses 10 in each embodiment may be an XR (AR: augmented reality, VR: virtual reality, MR: mixed reality) glasses-type information device such as smart glasses or a head-mounted display: Further, the variable focus glasses 10 according to each modification may be an XR glasses-type information device such as smart glasses or a head-mounted display.

Further, the optical elements such as the liquid crystal elements LU1, LF1, LX1, the prism element P1, the variable focus lens LN, and the variable prism VP described in the first to fifth embodiments and their modifications may be applied as an optical component of an illumination optical system or a projection optical system.

Further, when the variable focus eyeglasses 10 in the first embodiment and the like are glasses-type information equipment that includes an image display device that covers the front of the wearer's eyes or an image display device that displays an image superimposed on the real world, it is desirable that the variable focus eyeglasses 10 can be used even with these image display devices removed. In this case, regardless of whether the image display device is removed or not, the refractive index distribution of the variable focus lens LN is controlled based on the information acquired by the gaze information acquisition section LS.

Note that when the user of the eyeglass system 1 as in the first embodiment above uses a small device such as a mobile device, there is a possibility that the mode cannot be switched to a mode that is comfortable for the user. For example, a case may occur where detection by a laser ranging module or the like becomes insufficient. Possible. Therefore, for example, when performing an operation of detecting a signal from a predetermined device owned by the user, the power setting switching unit SW may be controlled to be fixed to one of the first to third modes. Furthermore, when performing the above-mentioned detection operation, the power setting switching unit SW may switch to another mode in which the power setting is different from the first to third modes. Specifically, the following software is installed in advance in a device in which mutual communication between the variable focus eyeglasses 10 and Bluetooth or the like is ensured. This software is software that detects the working state (active state) or operating state of the device and outputs a detection signal indicating the state to the variable focus eyeglasses 10. Then, when the detection signal is obtained in the variable focus eyeglasses 10, the power setting switching unit SW may be fixed to a power setting corresponding to the short distance mode.

Further, the present invention is not limited to the embodiments described above, and various modifications can be made without departing from the gist thereof. For example, the configurations described in the above embodiments can be replaced with substantially the same configurations, configurations that provide the same effects, or configurations that can achieve the same objectives.

### EXPLANATION OF SYMBOLS

1 eyeglass system, 10 variable focus eyeglasses, 15 smart glasses, 16 head-mounted display, 20 operation device, 30 server device, 40 client device for medical personnel, 50 medical-related database, NT network. 101 rim, 103 temple, 105 bridge, D1 nose pad, EX external unit, LU1 to LU6 liquid crystal element, LF1 to LF6 liquid crystal element, LX1 to LX4 liquid crystal element, P1, P2 prism element, B1, B2 transparent substrate, LC liquid crystal layer, LN variable focus lens, VP variable prism, U1 unit-electrode, CT center electrode, CC core electrode, LA optical axis, RF refractive index distribution, E1 first electrode, E2 second electrode, E3 counter electrode, EC auxiliary electrode, W1 first lead wire, W2 2nd lead wire, W3 3rd lead wire, W4 4th lead wire, W5 5th lead wire, W6 6th lead wire, W7 7th lead wire, WS Lead wire area, LR Lens area, HR resistive layer, AR area, IS1, IS2 insulating layer, C1, C1a˜C1d, C2a˜C2d, C3a˜C3d common input section, Sa˜Sh Fan-shaped area, LS gaze information acquisition unit, LI gaze space information acquisition unit, LM gaze behavior detection unit, UM discomfort behavior detection unit, TD attention distance deriving unit, DC power control unit, MV update control unit, DM power model information storage unit, PM prism control information storage unit, SW power setting switching unit, 11, 21 Transmission unit, 12, 22 Receiving unit, 13 Storage unit, 23 Display unit, FC lens control unit, MC control unit, AX1 first control unit, AX2 second control unit, VC prism control unit, 31 data acquisition unit, 32 model generation unit, 33 prediction unit, 34 storage unit, 34a lifestyle information storage unit, 34b diagnostic information storage unit, 34c learning data storage unit, 34d model storage unit, MC1 display control means, MC2 operation acceptance means, MC3 power setting support mode execution means, 34e power setting Information storage unit, 34f user-related information storage unit, 34g optometry information storage unit, 34h power model information storage unit, RG1, RG2, RG3 first to third annular regions, CB1, CB2, CX1, CX2, PB1, PB2 transparent substrate, CLD, CLX, PLC liquid crystal layer.

The invention claimed is:

1. An optical element comprising a plurality of liquid crystal elements,
each of the plurality of liquid crystal elements comprising:
a pair of flat transparent substrates;
a liquid crystal layer sealed between the transparent substrates;
unit-electrodes including linear first and second electrodes formed on one of the flat transparent substrates via a resistive layer and an insulating layer; and
a common electrode formed on the other of the flat transparent substrate, and
refracting light passing through the liquid crystal layer by generating a retardation gradient in the liquid crystal layer between the first and second electrodes by supplying a control voltage to the unit-electrodes and the common electrode,
wherein the optical element is configured by superimposing at least a first liquid crystal unit including first and second liquid crystal elements of the plurality of liquid crystal elements, and a second liquid crystal unit including third and fourth liquid crystal elements of the plurality of liquid crystal elements,
each of the unit-electrodes in the first to fourth liquid crystal elements has a predetermined width between the first and second electrodes, extends linearly, and is arranged in plurality to form a plurality of unit-electrodes, and
the first liquid crystal unit is configured such that an extension direction of each of the unit-electrodes in the first and second liquid crystal elements is set to a first direction and alignment directions of the liquid crystal layer in the first and second liquid crystal elements are perpendicular to each other, and
the second liquid crystal unit is configured such that an extension direction of each of the unit-electrodes in the third and fourth liquid crystal elements is set to a second direction different from the first direction, and alignment directions of the liquid crystal layer in the third and fourth liquid crystal elements are perpendicular to each other, and
the optical element is configured to form a Fresnel lens-like refractive index distribution having an optical axis, by generating a retardation gradient having a focal axis in the first direction in the first liquid crystal unit and a retardation gradient having a focal axis in the second direction in the second liquid crystal unit, when an individual control voltage is supplied to each of the first and second electrodes of the unit-electrodes in the first liquid crystal unit and the second liquid crystal unit.

2. The optical element according to claim 1, wherein at least a portion of the plurality of retardation gradients generated between the unit-electrodes is controlled based on a desired lens function when a voltage that is high or low with respect to a threshold voltage of the liquid crystal layer is supplied as the control voltage to one of the first and second electrodes on the low potential side.

3. The optical element according to claim 1, wherein the optical axis is formed in a region formed by intersecting any pair of adjacent unit electrodes when a voltage, which produces a retardation gradient symmetrical with respect to a boundary portion between the pair of adjacent unit-electrodes, is applied as the control voltage to the pair of adjacent unit electrodes among the plurality of unit-electrodes of the first to fourth liquid crystal elements.

4. The optical element according to claim 1, wherein the optical axis is formed at a central portion where any one of the unit-electrodes intersects when an equal voltage is supplied as the control voltage to the first electrode and the second electrode in any one of the plurality of unit-electrodes.

5. The optical element according to claim 1, wherein image quality outside an arrangement area is degraded when the control voltage applied between the unit-electrodes located outside the arrangement area among the plurality of unit-electrodes in the first to fourth liquid crystal elements are controlled to be different in any one or all of voltage value, frequency, and phase from the desired control voltage applied to the unit-electrodes inside the arrangement area.

6. The optical element according to claim 1, wherein at least one of the first and second liquid crystal units refracts light passing through the liquid crystal layer in a fixed direction, causing at least one of the first and second liquid crystal units to function as a variable prism for controlling the deflection angle of the light when a voltage for controlling the retardation gradients generated in the respective unit-electrodes to be the same gradient is supplied as the control voltage.

7. The optical element according to claim 1, wherein the predetermined width between the first and second electrodes in the plurality of unit-electrodes is set to be substantially the same.

8. The optical element according to claim 1, further comprising:
a third liquid crystal unit superimposed thereon,
wherein the third liquid crystal unit comprises a pair of liquid crystal elements, each of the pair of liquid crystal elements including:
a pair of flat transparent substrates;
a liquid crystal layer sealed between the transparent substrates;
a plurality of unit-electrodes formed on one of the transparent substrates and including a plurality of center electrodes, the plurality of unit-electrodes including a first arc-shaped electrode and a second arc-shaped electrode arranged in each of a plurality of annular regions divided radially and circumferentially with respect to an optical axis, and the plurality of center electrodes including a core electrode corresponding to the first arc-shaped electrode formed in a portion of the one of the transparent substrates through which the optical axis passes and the second arc-shaped electrode arranged concentrically with respect to the optical axis; and
a common electrode formed on the other transparent substrate, and
the third liquid crystal unit is formed by superimposing two of the liquid crystal elements with orientation directions of the respective liquid crystal layers perpendicular to each other, and is configured to form a desired Fresnel lens-like refractive index distribution in the liquid crystal layer when a control voltage is supplied to the plurality of unit-electrodes, the core electrode, and the common electrode.

9. The optical element according to claim 8, wherein the first and second liquid crystal units are arranged so that a narrow angle formed by the first and second directions, which are extending directions of the plurality of unit-electrodes, is approximately 45 degrees, or 20 degrees or more and 70 degrees or less, preferably 30 degrees or more and 60 degrees or less.

10. The optical element according to claim 1, further comprising:
at least a fourth liquid crystal unit having the same configuration as the first and second liquid crystal units is provided in an overlapping manner.

11. An optical element comprising a plurality of liquid crystal elements,
each of the plurality of liquid crystal elements comprising:
a pair of flat transparent substrates;
a liquid crystal layer sealed between the transparent substrates;
unit-electrodes including linear first and second electrodes formed on one of the flat transparent substrates via a resistive layer and an insulating layer; and
a common electrode formed on the other of the flat transparent substrate, and
refracting light passing through the liquid crystal layer by generating a retardation gradient in the liquid crystal layer between the first and second electrodes by supplying a control voltage to the unit-electrodes and the common electrode,
wherein the optical element is configured by superimposing at least a fifth liquid crystal unit including fifth and sixth liquid crystal elements of the plurality of liquid crystal elements, and a sixth liquid crystal unit including seventh and eighth liquid crystal elements of the plurality of liquid crystal elements; and
each of the unit-electrodes in the fifth to eighth liquid crystal elements has one core electrode formed in an approximate center of an arrangement area formed and arranged on the transparent substrate, which corresponds to the first electrode in the unit-electrodes, and the unit electrodes are configured as a plurality of unit-electrodes by arranging the second electrode and the first electrode in that order on both sides of the core electrode as the center, and a width between the first and second electrodes in the unit electrodes is narrowed toward both ends of the arrangement area; and
the fifth liquid crystal unit is configured such that an extension direction of each of the unit-electrodes in the fifth and sixth liquid crystal elements is set to a first direction and alignment directions of the liquid crystal layer in the fifth and sixth liquid crystal elements are perpendicular to each other; and
the sixth liquid crystal unit is configured such that an extension direction of each of the unit-electrodes in the seventh and eighth liquid crystal elements is set to a second direction different from the first direction, and alignment directions of the liquid crystal layer in the seventh and eighth liquid crystal elements are perpendicular to each other; and
the optical element is configured to form a Fresnel lens-like refractive index distribution having an optical axis, generating a retardation gradient having a focal axis in the first direction in the fifth liquid crystal unit and a retardation gradient having a focal axis in the second direction in the sixth liquid crystal unit when an individual control voltage is supplied to each of the first and second electrodes of the plurality of unit-electrodes in the fifth liquid crystal unit and the sixth liquid crystal unit.

12. The optical element according to claim 11, further comprising:
a seventh liquid crystal unit having the same configuration as the fifth and sixth liquid crystal units; and
each of the fifth to seventh liquid crystal units is configured such that the outer shape and the region in which the Fresnel lens-like refractive index distribution is formed are arranged so that they substantially match each other even when rotated at an appropriate angle.

13. An optical element comprising:
a variable prism made of a pair of liquid crystal prism elements; and
a variable focus lens made of a pair of liquid crystal elements, superimposed on the variable prism,
wherein each of the pair of liquid crystal prism elements comprises:
a pair of flat transparent substrates;
a liquid crystal layer sealed between the transparent substrates;
a unit-electrode including linear first and second electrodes formed on one of the flat transparent substrates via a resistive layer and an insulating layer; and
a common electrode formed on the other of the flat transparent substrate, and
each of the pair of liquid crystal prism elements refracts light passing through the liquid crystal layer in a fixed direction by supplying a control voltage to the first and second electrodes and the common electrode to generate a retardation gradient in the liquid crystal layer between the first and second electrodes, and the variable prism is formed by superimposing the liquid crystal prism elements, and
each of the pair of liquid crystal elements comprises:
a pair of flat transparent substrates;
a liquid crystal layer sealed between the transparent substrates;
a plurality of unit-electrodes formed on one of the transparent substrates and including a plurality of center electrodes, the plurality of unit-electrodes including a first arc-shaped electrode and a second arc-shaped electrode arranged in each of a plurality of annular regions divided radially and circumferentially with respect to an optical axis, and the plurality of center electrodes including a core electrode corresponding to the first arc-shaped electrode formed in a portion of the one of the transparent substrates through which the optical axis passes and the second arc-shaped electrode arranged concentrically with respect to the optical axis; and
a common electrode formed on the other of the transparent substrates, and
wherein the variable focus lens is formed by superimposing the pair of liquid crystal elements with orientation directions of the respective liquid crystal layer in the pair of liquid crystal elements being perpendicular to each other, and is configured to form a desired Fresnel lens-like refractive index distribution in the liquid crystal layer when a control voltage is supplied to the plurality of unit-electrodes, the common electrode, and the plurality of center electrodes.

14. Eyeglasses comprising the optical element according to claim 1.

15. Eyeglasses comprising the optical element according to claim 11.

16. Eyeglasses comprising the optical element according to claim 13.
